SEQUENCE LISTING (55 sequences)

SEQ ID NO: 1 - protein, 6 aa, synthetic construct
HHHHHH

SEQ ID NO: 2 - protein, 128 aa, Homo sapiens
GRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 3 - protein, 173 aa, Homo sapiens
EPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 4 - protein, 100 aa, Homo sapiens
YDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 5 - protein, 216 aa, Homo sapiens
MSVPRNRIGVGVIDGHIYAVGGSHGCIHHNSVERYEPERDEWHLVAPMLTRRIGVGVAVLNRLLYAVGGFDGTNRLNSAECYYPERNEWRMITAMNTIRSGAGVCVLHNCIYAAGGYDGQDQLNSVERYDVETETWTFVAPMKHRRSALGITVHQGRIYVLGGYDGHTFLDSVECYDPDTDTWSEVTRMTSGRSGVGVAVTMEPCRKQIDQQNCTC

SEQ ID NO: 6 - protein, 14 aa, synthetic construct
RPRSWLELPLRLAD

SEQ ID NO: 7 - protein, 15 aa, synthetic construct
PGTHLAKILSDMRSQ

SEQ ID NO: 8 - protein, 15 aa, synthetic construct
ERRVANNARELLRVR

SEQ ID NO: 9 - protein, 17 aa, synthetic construct
LQRFHYSKVNKSDDTAE

SEQ ID NO: 10 - protein, 16 aa, synthetic construct
PAGIRGPYMFQWNDDR

SEQ ID NO: 11 - protein, 16 aa, synthetic construct
IKTDWITSFYKSSLKD

SEQ ID NO: 12 - protein, 16 aa, synthetic construct
AKERARVADSNRQLAL

SEQ ID NO: 13 - protein, 15 aa, synthetic construct
RFLNSFASMLRFGPI

SEQ ID NO: 14 - protein, 15 aa, synthetic construct
KRLARVLSVDSGTDV

SEQ ID NO: 15 - protein, 15 aa, synthetic construct
ALDSFLRLNDVQFTV

SEQ ID NO: 16 - protein, 15 aa, synthetic construct
TRNVDVQLLDTKRQL

SEQ ID NO: 17 - protein, 16 aa, synthetic construct
ETNRARILAPLLIVDK

SEQ ID NO: 18 - protein, 16 aa, synthetic construct
SRHLALSWPTFGIIVK

SEQ ID NO: 19 - protein, 15 aa, synthetic construct
KRPCPWSEPSVGTTL

SEQ ID NO: 20 - protein, 16 aa, synthetic construct
TVPIYEVYALPHAILR

SEQ ID NO: 21 - protein, 15 aa, synthetic construct
TVEAEAAHVTVTRHY

SEQ ID NO: 22 - protein, 17 aa, synthetic construct
QQSALRVMQWNCAAYLK

SEQ ID NO: 23 - protein, 15 aa, synthetic construct
RVFNNVLLQQTQRLD

SEQ ID NO: 24 - protein, 15 aa, synthetic construct
PVPEGAGLKGHLHKL

SEQ ID NO: 25 - protein, 14 aa, Homo sapiens
RPRSWRELPLRLAD

SEQ ID NO: 26 - protein, 15 aa, Homo sapiens
PGTDLAKILSDMRSQ

SEQ ID NO: 27 - protein, 15 aa, Homo sapiens
ERRVANNARERLRVR

SEQ ID NO: 28 - protein, 17 aa, Homo sapiens
LQRFLYSKVNKSDDTAE

SEQ ID NO: 29 - protein, 16 aa, Homo sapiens
PAGIGGPYMFQWNDDR

SEQ ID NO: 30 - protein, 16 aa, Homo sapiens
IKTVWITSFYKSSLKD

SEQ ID NO: 31 - protein, 16 aa, Homo sapiens
AKERARVADSDRQLAL

SEQ ID NO: 32 - protein, 15 aa, Homo sapiens
RFLNSFASMHRFGPI

SEQ ID NO: 33 - protein, 15 aa, Homo sapiens
KRHARVLSVDSGTDV

SEQ ID NO: 34 - protein, 15 aa, Homo sapiens
ALDSFLRLNDGQFTV

SEQ ID NO: 35 - protein, 15 aa, Homo sapiens
TRNLDVQLLDTKRQL

SEQ ID NO: 36 - protein, 16 aa, Homo sapiens
ETNRARILAPLLILDK

SEQ ID NO: 37 - protein, 16 aa, Homo sapiens
SRHLALSGPTFGIIVK

SEQ ID NO: 38 - protein, 15 aa, Homo sapiens
KRPGPWSEPSVGTTL

SEQ ID NO: 39 - protein, 16 aa, Homo sapiens
TVPIYEGYALPHAILR

SEQ ID NO: 40 - protein, 15 aa, Homo sapiens
TVEAEAAHGTVTRHY

SEQ ID NO: 41 - protein, 17 aa, Homo sapiens
QQSALRVMQRNCAAYLK

SEQ ID NO: 42 - protein, 15 aa, Homo sapiens
RVFNNVLLQQTQHLD

SEQ ID NO: 43 - protein, 15 aa, Homo sapiens
PVPEGAGLKGHLPKL

SEQ ID NO: 44 - protein, 21 aa, synthetic construct
KRQQQQSALRVMQWNCAAYLK

SEQ ID NO: 45 - protein, 21 aa, synthetic construct
YLADQCWAIDDIVKTSVPSRE

SEQ ID NO: 46 - protein, 25 aa, synthetic construct
PEREEKLRLAEAGLSALPTPVPQQR

SEQ ID NO: 47 - protein, 18 aa, synthetic construct
AVFIENSCMEKGSKQGRN

SEQ ID NO: 48 - protein, 16 aa, synthetic construct
KYLLPNVTSLIQPMSQ

SEQ ID NO: 49 - protein, 21 aa, Homo sapiens
KRQQQQSALRVMQRNCAAYLK

SEQ ID NO: 50 - protein, 21 aa, Homo sapiens
YLADQWWAIDDIVKTSVPSRE

SEQ ID NO: 51 - protein, 18 aa, Homo sapiens
AVCIENSCMEKGSKQGRN

SEQ ID NO: 52 - protein, 16 aa, Homo sapiens
KYLPPNVTSLIQPMSQ

SEQ ID NO: 53 - DNA, 13 bp, synthetic construct
  source 1..13: mol_type = other DNA
ttcaagtcaggtt

SEQ ID NO: 54 - DNA, 13 bp, synthetic construct
  source 1..13: mol_type = other DNA
gaggccgtcatct

SEQ ID NO: 55 - DNA, 13 bp, synthetic construct
  source 1..13: mol_type = other DNA
atcgtatccagac

(19) United States
(12) Patent Application Publication
(10) Pub. No.: US 2026/0034201 A1
(43) Pub. Date: Feb. 5, 2026

(54) COMBINATION THERAPY WITH NEOANTIGEN VACCINE

(71) Applicant: BioNTech US Inc., Cambridge, MA (US)

(72) Inventors: Kristen N. Balogh, Somerville, MA (US); Lakshmi Srinivasan, Lexington, MA (US); Richard Gaynor, Carmel, IN (US); Joong Hyuk Sheen, Brookline, MA (US); Ekaterina Esaulova, Arlington, MA (US)

(21) Appl. No.: 18/996,152

(22) PCT Filed: Jul. 20, 2023
(86) PCT No.: PCT/US2023/070550

§ 371 Date: Jan. 17, 2025

Related U.S. Application Data
(60) Provisional application No. 63/368,963, filed on Jul. 20, 2022.

(51) Int. Cl. A61K 39/00; A61K 31/282; A61K 39/39; A61K 39/395; A61K 40/11; A61K 40/32; A61P 35/00

(52) U.S. Cl. CPC A61K 39/0011; A61K 31/282; A61K 39/39; A61K 39/39558; A61K 40/11; A61K 40/32; A61P 35/00; A61K 2039/505; A61K 2039/545; A61K 2039/55561

(57) ABSTRACT

The present disclosure relates to neoplasia vaccine or immunogenic composition administered in combination with other agents, such as checkpoint blockade inhibitors for the treatment or prevention of neoplasia in a subject

### CROSS REFERENCE

This application is a 371 U.S. National Phase Application of International Application No. PCT/US2023/070550, filed Jul. 20, 2023 which claims the benefit of U.S. Provisional Application No. 63/368,963, filed on Jul. 20, 2022, which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 4, 2025, is named 50401-765_601_SL.xml and is 49,358 bytes in size.

### BACKGROUND

Cancer immunotherapy is the use of the immune system to treat cancer. Immunotherapies exploit the fact that cancer cells often have molecules on their surface that can be detected by the immune system, known as tumor antigens, which are often proteins or other macromolecules (e.g., carbohydrates). Active immunotherapy directs the immune system to attack tumor cells by targeting tumor antigens. Passive immunotherapies enhance existing anti-tumor responses and include the use of monoclonal antibodies, lymphocytes and cytokines. Tumor vaccines are typically composed of tumor antigens and immunostimulatory molecules (e.g., adjuvants, cytokines or TLR ligands) that work together to induce antigen-specific cytotoxic T cells (CTLs) that recognize and lyse tumor cells. One of the critical barriers to developing curative and tumor-specific immunotherapy is the identification and selection of highly specific and restricted tumor antigens to avoid autoimmunity.

Tumor neoantigens, which arise as a result of genetic change (e.g., inversions, translocations, deletions, missense mutations, splice site mutations, etc.) within malignant cells, represent the most tumor-specific class of antigens and can be patient-specific or shared. Tumor neoantigens are unique to the tumor cell as the mutation and its corresponding protein are present only in the tumor. They also avoid central tolerance and are therefore more likely to be immunogenic. Therefore, tumor neoantigens provide an excellent target for immune recognition including by both humoral and cellular immunity. Accordingly, there is still a need for developing additional cancer therapeutics.

### SUMMARY

Provided herein is a method of treating or preventing a cancer in a human subject in need thereof comprising administering to the human subject in need thereof: (a) a first component comprising: (i) a polypeptide comprising a cancer-specific neoepitope of a protein expressed by cancer cells of the cancer, (ii) a polynucleotide encoding the polypeptide of (i), (iii) one or more APCs comprising the polypeptide of (i) or the polynucleotide of (ii), (iv) a T cell receptor (TCR) specific for a complex comprising an HLA protein expressed by the human subject and a cancer-specific neoepitope of a protein expressed by cancer cells of the cancer neoepitope, or (v) T cells comprising the TCR of (iv); and (b) a second component comprising an anti-cancer agent which is an antibody or an antigen-binding portion thereof that binds specifically to a Programmed Death-1 (PD-1) receptor and inhibits PD-1 activity; and (c) a third component comprising a platinum-based chemotherapy; wherein the human subject: (i) has not previously received a systemic treatment for metastatic disease, (ii) has not previously received an immunotherapy with an anti-PD-1 antibody, and (iii) has not previously received an immunotherapy with an anti-PD-L1 antibody.

In some embodiments, the method comprises administering to the human subject a combination of the second component and the third component prior to administering the first component.

In some embodiments, the method comprises administering to the human subject a combination of the second component and the third component for a period of 12 weeks prior to administering the first component. In some embodiments, manufacturing of the first component takes place during the period of 12 weeks in which the combination of the second component and the third component are administered. In some embodiments, the method comprises administering the first component for a period of 12 weeks after administering the combination of the second component and the third component for a period of 12 weeks. In some embodiments, the administering the first component for a period of 12 weeks after administering the combination of the second component and the third component for a period of 12 weeks comprises administering the first component at four separate anatomical locations of the human subject. In some embodiments, the administering the first component for a period of 12 weeks after administering the combination of the second component and the third component for a period of 12 weeks comprises administering five priming does of the first component and two booster doses of the first component. In some embodiments, the administering the first component for a period of 12 weeks after administering the combination of the second component and the third component for a period of 12 weeks comprises administering a priming dose of the first component on days 1 and 4 and then weekly in weeks 13, 14, and 15; and administering a boosting dose in weeks 19 and 23. In some embodiments, the second component is administered to the human subject during the period of 12 weeks in which the first component is administered. In some embodiments, the second component is administered to the human subject after the period of 12 weeks in which the first component and second component are administered. In some embodiments, the second component is administered to the human subject for a period of at least 28 weeks after the period of 12 weeks in which the first component and second component are administered. In some embodiments, the second component is administered to the human subject for a period of 80 weeks after the period of 12 weeks in which the first component and second component are administered. In some embodiments, the second component is administered to the human subject for a total period of at least 52 weeks or about 103 or about 104 weeks. In some embodiments, the third component is not administered to the human subject during or after administration of the first component. In some embodiments, the third component is not administered to the human subject following administration of the combination of the second component and the third component for the period of 12 weeks prior to administering the first component.

In some embodiments, the human subject has a KRAS mutation, a TP53 mutation, and/or a KEAP1 mutation. In some embodiments, the cancer-specific neoepitope of the first component does not comprise a KRAS neoepitope, a TP53 neoepitope, and/or a KEAP1 neoepitope.

In some embodiments, the cancer is a lung cancer.

In some embodiments, the lung cancer is non-small cell lung cancer (NSCLC).

In some embodiments, the NSCLC has a squamous histology.

In some embodiments, the NSCLC has a non-squamous histology.

In some embodiments, the NSCLC is metastatic NSCLC.

In some embodiments, the first component comprises the polypeptide comprising a cancer-specific neoepitope of a protein expressed by cancer cells of the cancer.

In some embodiments, the first component comprises an adjuvant.

In some embodiments, the adjuvant comprises poly I:poly C.

In some embodiments, the cancer-specific neoepitope comprises at least two different cancer-specific neoepitopes of a protein expressed by cancer cells of the cancer.

In some embodiments, the cancer-specific neoepitope comprises at most twenty different cancer-specific neoepitopes of a protein expressed by cancer cells of the cancer.

In some embodiments, the method comprises comparing: (i) nucleic acid sequences obtained by whole genome sequencing or whole exome sequencing of cancer cells from the single subject to (ii) nucleic acid sequences obtained by whole genome sequencing or whole exome sequencing of non-cancer cells from the single subject. In some embodiments, the method comprises identifying a plurality of cancer specific nucleic acid sequences that are unique to cancer cells of the human subject based on the comparing.

In some embodiments, the method comprises predicting or calculating binding affinities of cancer-specific neoepitope sequences encoded by the identified plurality of cancer specific nucleic acid sequences to a protein encoded by an HLA allele of the human subject by an HLA peptide binding analysis using a program implemented on a computer system.

In some embodiments, the method comprises selecting at least two cancer-specific neoepitope sequences predicted or calculated to have an IC₅₀ to a protein encoded by an HLA allele of the human subject with of less than 500 nM or 150 nM or less.

In some embodiments, the anti-PD-1 antibody or antigen-binding portion thereof cross-competes with nivolumab for binding to human PD-1.

In some embodiments, the anti-PD-1 antibody or antigen-binding portion thereof comprises a heavy chain constant region which is of a human IgG1 or IgG4 isotype.

In some embodiments, the anti-PD-1 antibody or antigen-binding portion thereof is a chimeric, humanized or human monoclonal antibody or a portion thereof.

In some embodiments, the anti-PD-1 antibody is pembrolizumab.

The method, wherein the anti-PD-1 antibody or antigen-binding portion thereof is administered at a dose ranging from 0.1 to 10.0 mg/kg body weight once every 2, 3 or 4 weeks.

In some embodiments, the anti-PD-1 antibody or antigen-binding portion thereof is administered at a dose of 5 or 10 mg/kg body weight once every 3 weeks.

In some embodiments, the anti-PD-1 antibody or antigen-binding portion thereof is administered at a dose of 3 mg/kg body weight once every 2 weeks.

In some embodiments, the anti-PD-1 antibody or antigen-binding portion thereof is administered via intravenous infusion at a dose of 200 mg on cycle day 1 every 3 weeks.

In some embodiments, the platinum-based chemotherapy is a platinum-based doublet chemotherapy (PT-DC).

In some embodiments, the PT-DC is a combination of pemetrexed and carboplatin.

In some embodiments, the carboplatin is administered at a dose to achieve an area under the free carboplatin plasma concentration versus time curve (AUC) of 5.

In some embodiments, the pemetrexed is administered at a dose of 500 mg/m².

In some embodiments, the PT-DC was administered concurrently with the anti-PD-1 antibody or antigen-binding portion thereof for 4 doses of the anti-PD-1 antibody or antigen-binding portion thereof, followed by repeated administration of the anti-PD-1 antibody or antigen-binding portion thereof alone.

In some embodiments, the method promotes epitope spread.

In some embodiments, the method promotes epitope spread of an epitope that is different than any of the cancer-specific neoepitopes.

In some embodiments, the epitope that is different than any of the cancer-specific neoepitopes comprises a KRAS neoepitope, a TP53 neoepitope, and/or a KEAP1 neoepitope.

In some embodiments, the KRAS neoepitope comprises a G12C or G12V mutation.

In some embodiments, a median progression-free survival (PFS) of a first population of human subjects with the cancer treated with the first, second and third components is longer than a median PFS of a second population of subjects with the cancer treated with the second and/or third components but not the first component.

In some embodiments, an overall response rate (ORR) of a first population of human subjects with the cancer treated with the first, second and third components is higher than an ORR of a second population of subjects with the cancer treated with the second and/or third components but not the first component.

In some embodiments, a percentage of subjects with at least a 9-month progression-free survival (PFS) of a first population of human subjects with the cancer treated with the first, second and third components is higher than a percentage of subjects with at least a 9-month PFS of a second population of subjects with the cancer treated with the second and/or third components but not the first component.

In some embodiments, a percentage of subjects with at least a 12-month progression-free survival (PFS) of a first population of human subjects with the cancer treated with the first, second and third components is higher than a percentage of subjects with at least a 12-month PFS of a second population of subjects with the cancer treated with the second and/or third components but not the first component.

In some embodiments, a median overall survival (OS) of a first population of human subjects with the cancer treated with the first, second and third components is longer than a median OS of a second population of subjects with the cancer treated with the second and/or third components but not the first component.

In some embodiments, a percentage of subjects who achieve complete response, partial response, prolonged stable disease or stable disease for 6 months or more (CBR) of a first population of human subjects with the cancer treated with the first, second and third components is higher than an CBR of a second population of subjects with the cancer treated with the second and/or third components but not the first component.

In some embodiments, a reduction of tumor size in a first population of human subjects with the cancer treated with the first, second and third components is greater than reduction of tumor size in a second population of subjects with the cancer treated with the second and/or third components but not the first component.

In some embodiments, a level of CD4+ T cells that infiltrate a tumor in a first population of human subjects treated with the first, second and third components is higher than a level of CD4+ T cells that infiltrate a tumor in a second population of subjects treated with the second and/or third components but not the first component.

In some embodiments, a level of effector and cytotoxic CD4+ T cells generated in a first population of human subjects treated with the first, second and third components is higher than a level of effector and cytotoxic CD4+ T cells generated in a second population of subjects treated with the second and/or third components but not the first component. In some embodiments, the method increases a level of CD4+ T cells specific to a cancer-specific neoepitope that upregulate expression of ZEB2, PDCD1, TOX, TIGT, CXCR3, ITGB1, GZMA and/or ICOS.

In some embodiments, the method increases a level of CD4+/CD62L^{hi}/CD69+/CD27+/CCR7+ T cells specific to a cancer-specific neoepitope.

In some embodiments, the method increases a level of CD4+/NKG7+/CCL4+/CCL5+/GNLY+/LAG3+ T cells specific to a cancer-specific neoepitope.

In some embodiments, the human subject is identified as having a PD-L1-positive cancer prior to administration of the first, second and/or third components.

In one aspect, provided herein is a method of treating or preventing a cancer in a human subject in need thereof comprising administering to the human subject in need thereof: (a) a first component comprising: (i) a polypeptide comprising a cancer-specific neoepitope of a protein expressed by cancer cells of the cancer, (ii) a polynucleotide encoding the polypeptide of (i), (iii) one or more APCs comprising the polypeptide of (i) or the polynucleotide of (ii), (iv) a T cell receptor (TCR) specific for a complex comprising an HLA protein expressed by the human subject and a cancer-specific neoepitope of a protein expressed by cancer cells of the cancer neoepitope, or (v) T cells comprising the TCR of (iv); and (a) a second component comprising an anti-cancer agent which is an antibody or an antigen-binding portion thereof that binds specifically to a Programmed Death-1 (PD-1) receptor and inhibits PD-1 activity; and (b) a third component comprising a platinum-based chemotherapy; wherein: (i) the method promotes epitope spread of an epitope that is different than any of the cancer-specific neoepitopes; (ii) a median progression-free survival (PFS) of a first population of human subjects with the cancer treated with the first, second and third components is longer than a median PFS of a second population of subjects with the cancer treated with the second and/or third components but not the first component; (iii) an overall response rate (ORR) of a first population of human subjects with the cancer treated with the first, second and third components is higher than an ORR of a second population of subjects with the cancer treated with the second and/or third components but not the first component; (iv) a percentage of subjects with at least a 9 or 12-month progression-free survival (PFS) of a first population of human subjects with the cancer treated with the first, second and third components is higher than a percentage of subjects with at least a 9 or 12-month PFS of a second population of subjects with the cancer treated with the second and/or third components but not the first component; (v) a median overall survival (OS) of a first population of human subjects with the cancer treated with the first, second and third components is longer than a median OS of a second population of subjects with the cancer treated with the second and/or third components but not the first component; (vi) a percentage of subjects who achieve complete response, partial response, prolonged stable disease or stable disease for 6 months or more (CBR) of a first population of human subjects with the cancer treated with the first, second and third components is higher than an CBR of a second population of subjects with the cancer treated with the second and/or third components but not the first component; (vii) reduction of tumor size in a first population of human subjects with the cancer treated with the first, second and third components is greater than reduction of tumor size in a second population of subjects with the cancer treated with the second and/or third components but not the first component; (viii) a level of CD4+ T cells that infiltrate a tumor in a first population of human subjects treated with the first, second and third components is higher than a level of CD4+ T cells that infiltrate a tumor in a second population of subjects treated with the second and/or third components but not the first component; (ix) a level of effector and cytotoxic CD4+ T cells generated in a first population of human subjects treated with the first, second and third components is higher than a level of effector and cytotoxic CD4+ T cells generated in a second population of subjects treated with the second and/or third components but not the first component; (x) the method increases a level of CD4+ T cells specific to a cancer-specific neoepitope that upregulate expression of ZEB2, PDCD1, TOX, TIGT, CXCR3, ITGB1, GZMA and/or ICOS; (xi) the method increases a level of CD4+/CD62L^{hi}/CD69+/CD27+/CCR7+ T cells specific to a cancer-specific neoepitope; and/or (xii) the method increases a level of CD4+/NKG7+/CCL4+/CCL5+/GNLY+/LAG3+ T cells specific to a cancer-specific neoepitope.

In some embodiments, the human subject is identified as having a PD-L1-positive cancer prior to administration of the first, second and/or third components.

In some embodiments, the human subject has not previously received a systemic treatment for metastatic disease, has not previously received an immunotherapy with an anti-PD-1 antibody, and/or has not previously received an immunotherapy with an anti-PD-L1 antibody.

In some embodiments, provided herein is a method of treating or preventing a neoplasia in a human subject in need thereof comprising administering to a subject in need thereof: a first component comprising (i) a peptide comprising a neoepitope of a protein, (ii) a polynucleotide encoding the peptide, (iii) one or more APCs comprising the peptide or the polynucleotide encoding the peptide or (iv) a T cell receptor (TCR) specific for the neoepitope in complex with an HLA protein; and a second component comprising at least two therapeutics e.g., anti-PD-L1 monoclonal antibody Pembrolizumab, Carboplatin and/or Pemetrexed.

In some embodiments, the first component comprises a neoplasia vaccine or immunogenic composition.

In some embodiments, the first component further comprises an adjuvant. In some embodiments, the adjuvant is poly-ICLC.

In some embodiments, first component comprises a neoplasia vaccine or immunogenic composition comprises neoantigenic peptides, wherein the peptides comprises at least two, at least three, at least four or at least five peptides. In some embodiments, the peptide comprises at most 15, at most 20, at most 25 or at most 30 peptides. In some embodiments, the peptide is from 5 to 50 amino acids in length. In some embodiments, the peptide is from 14 to 35 amino acids in length. In some embodiments, the neoepitope of each peptide is unique.

In some embodiments, the first component further comprises a pH modifier. In some embodiments, the first component further comprises a pharmaceutically acceptable carrier.

In some embodiments, the subject is suffering from a neoplasia selected from the group consisting of Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate. In some embodiments, the neoplasia is metastatic melanoma. In some embodiments, the subject has no detectable neoplasia but is at high risk for disease recurrence. In embodiments, the cancer is selected from the group consisting of: adrenal, bladder, breast, cervical, colorectal, glioblastoma, head and neck, kidney chromophobe, kidney clear cell, kidney papillary, liver, lung adenocarcinoma, lung squamous, ovarian, pancreatic, melanoma, stomach, uterine corpus endometrial, and uterine carcinosarcoma. In embodiments, the cancer is selected from the group consisting of: prostate cancer, bladder, lung squamous, NSCLC, breast, head and neck, lung adenocarcinoma, GBM, Glioma, CML, AML, supretentorial ependyomas, acute promyelocytic leukemia, solitary fibrous tumors, and crizotinib resistant cancer. In embodiments, the cancer is selected from the group consisting of: CRC, head and neck, stomach, lung squamous, lung adenocarcinoma, prostate, bladder, stomach, renal cell carcinoma, and uterine. In embodiments, the cancer is selected from the group consisting of: melanoma, lung squamous, DLBCL, uterine, head and neck, uterine, liver, and CRC. In embodiments, the cancer is selected from the group consisting of: lymphoid cancer; Burkitt lymphoma, neuroblastoma, prostate adenocarcinoma, colorectal adenocarcinoma; Uterine/Endometrium Adenocarcinoma; MSI+; endometrium serous carcinoma; endometrium carcinosarcoma-malignant mesodermal mixed tumour; glioma; astrocytoma; GBM, acute myeloid leukemia associated with MDS; chronic lymphocytic leukemia-small lymphocytic lymphoma; myelodysplastic syndrome; acute myeloid leukemia; luminal NS carcinoma of breast; chronic myeloid leukemia; ductal carcinoma of pancreas; chronic myelomonocytic leukemia; myelofibrosis; myelodysplastic syndrome; prostate adenocarcinoma; essential thrombocythaemia; and medullomyoblastoma. In embodiments, the cancer is selected from the group consisting of: colorectal, uterine, endometrial, and stomach. In embodiments, the cancer is selected from the group consisting of: cervical, head and neck, anal, stomach, Burkitt's lymphoma, and nasopharyngeal carcinoma. In embodiments, the cancer is selected from the group consisting of: bladder, colorectal, and stomach. In embodiments, the cancer is selected from the group consisting of: lung, CRC, melanoma, breast, NSCLC, and CLL. In embodiments, the subject is a partial or non-responder to checkpoint inhibitor therapy. In embodiments, the cancer is selected from the group consisting of: bladder urothelial carcinoma (BLCA), breast invasive carcinoma (BRCA), breast cancer, cervical squamous cell carcinoma and endocervical adenocarcinoma (CESC), chronic lymphocytic leukemia (CLL), colorectal cancer (CRC), glioblastoma multiforme (GBM), head and neck squamous cell carcinoma (HNSC), kidney renal papillary cell carcinoma (KIRP), liver hepatocellular carcinoma (LIHC), lung adenocarcinoma (LUAD), lung squamous cell carcinoma (LUSC), pancreatic adenocarcinoma (PAAD), Prostate Cancer, skin cutaneous melanoma (SKCM), stomach adenocarcinoma (STAD), thyroid adenocarcinoma (THCA), and uterine corpus endometrioid carcinoma (UCEC). In embodiments, the cancer is selected from the group consisting of: colorectal cancer, uterine cancer, endometrium cancer, stomach cancer, and Lynch syndrome. In embodiments, the cancer is an MSI+ cancer.

In some embodiments, the first component is administered before the second component. In some embodiments, the second component is administered before the first component. In some embodiments, the first component is administered on the same day as the second component. In some embodiments, the second component is administered before the first component. In some embodiments, administration of pembrolizumab is initiated before initiation of administration of the first component. In some embodiments, administration of pembrolizumab is initiated before initiation of administration of the carboplatin or pemetrexed. In some embodiments, administration of pembrolizumab is initiated before initiation of administration of carboplatin or pemetrexed. In some embodiments, administration of pembrolizumab is on the same day as the initial administration of the first component. In some embodiments, administration of carboplatin is initiated on the same day as the initial administration of the first component. In some embodiments, administration of nivolumab continues every 12-36 or more weeks after a first administration of pembrolizumab. In some embodiments, administration of nivolumab continues every 2, 3, 4, 6 or 8 weeks after the first administration of pembrolizumab. In some embodiments, administration of an inhibitor, such as a checkpoint inhibitor or chemotherapeutic agent, is initiated following tumor resection. In some embodiments, administration of the first component is in a prime boost dosing regimen.

In some embodiments, administration of the first component is at weeks 1, 2, 3 or 4 as a prime. In some embodiments, administration of the first component is at months 2, 3, 4 or 5 as a boost. In some embodiments, administration of the first component is at weeks 19, 20, 21, 22, 23 or 24 as a boost.

In some embodiments, the peptide is administered at an average dose level of about 300-500 μg/ml per peptide. In some embodiments, a total dose of the peptide administered is from 4-8 mg. In some embodiments, pembrolizumab is administered at a dose of from 200-260 mg.

In some embodiments, the first component and/or the second component is administered intravenously or subcutaneously.

In some embodiments, a dose of the peptide is divided into at least 2, at least 3, at least 4 or at least 5 sub-doses. In some embodiments, each sub-dose of the peptide comprises at least 4 or at least 5 peptides.

In some embodiments, each peptide is administered at a dose of from 200-400 μg. In some embodiments, each sub-dose is administered at a different location of the subject.

In some embodiments, the method further comprises administering of one or more additional agents. In some embodiments, the additional agents are selected from the group consisting of: chemotherapeutic agents, anti-angiogenesis agents and agents that reduce immune-suppression.

In some aspects, provided herein is a method of treating or preventing cancer in a human subject in need thereof that has been treated with pembrolizumab comprising administering to the subject one or more chemotherapeutic agent at a dose of from 1-95% a dosage of the chemotherapeutic agent normally administered in a monotherapy regimen.

In some aspects, provided herein is a method of treating or preventing cancer in a human subject in need thereof that has been treated with pembrolizumab at a dose of from 1-95% a dosage of the pembrolizumab normally administered in a monotherapy regimen, the method comprising administering to the subject one or more chemotherapeutic drugs, like at a dose of less than its normal dose as a monotherapy.

In some aspects, provided herein is a method of treating or preventing cancer in a human subject in need thereof that has been treated with pembrolizumab comprising administering to the subject: carboplatin at a dose of from 1-95% a dosage of the dose that is normally administered in a monotherapy regimen; and pemetrexed at a dose of less than the dose normally administered in a monotherapy regimen.

In some embodiments, the method further comprises administering to the subject at least five peptides each comprising a unique neoepitope of a protein at a dose of from 100-500 μg of each peptide.

In some aspects, provided herein is a composition comprising: a first component comprising (i) a peptide comprising a neoepitope of a protein, (ii) a polynucleotide encoding the peptide, (iii) one or more APCs comprising the peptide or the polynucleotide encoding the peptide or (iv) a T cell receptor (TCR) specific for the neoepitope in complex with an HLA protein; and a second component comprising at least two inhibitors, wherein the at least two inhibitors comprise: pembrolizumab and platinum based chemotherapeutic agent or pembrolizumab and pemetrexed, or pembrolizumab and platinum based chemotherapeutic agent and pemetrexed.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “figure” and “FIG.” herein), of which:

FIG. 1A-1C: NEO-PV-01 vaccine generation, clinical study design, and patient disposition.

FIG. 1A. Schematic for sequencing of patients' tumors, prediction of neoantigens restricted to Class I MHC molecules, and generation of the synthetic long peptides included in the personalized neoantigen vaccines. Figure discloses SEQ ID NOS 53-55, respectively, in order of appearance.

FIG. 1B. Treatment with pembrolizumab and pemetrexed plus carboplatin was initiated at week 0; NEO-PV-01 was then administered between weeks 12 and 24, with pembrolizumab continuing for up to 2 years.

FIG. 1C. Study patient disposition. Seventeen of the 38 patients (45%) in the ITT set were not vaccinated due to early study termination for reasons including: inability to manufacture vaccine due to inadequate tumor and/or insufficient number of neoantigens (10 patients), adverse events (2 patients), patient consent withdrawn (2 patients), progressive disease, an investigator decision, or administration of a study-prohibited concomitant medication (1 patient each).

FIG. 2A-2E: Rates and durability of responses following treatment with NEO-PV-01 plus chemotherapy and anti-PD-1.

FIG. 2A. Best radiographic change (%) in sum of target lesions for each patient who received at least one dose of vaccine (VAX set). The dark shaded narrow bars represent the best change pre-NEO-PV-01; the light shade wider bars represent the best overall change in study for patients who received at least 1 dose of NEO-PV-01 plus anti-PD-1. Red indicates progressive disease, gray indicates stable disease, and blue indicates partial response.

FIG. 2B. Radiographic changes (%) in target lesions after initiation of pembrolizumab treatment for each patient (colors are the same as in a). Included are 30 of 38 ITT patients who had at least one post baseline RECIST assessment.

FIG. 2C. Swimmer's plot summarizing all patients on study. Each bar represents one subject in the study, bar length represents time on study.

FIG. 2D Kaplan-Meier estimates of PFS (top) and OS (bottom) for both the ITT (left) and vaccinated (right) patient sets.

FIG. 2E Measurement of ctDNA in the peripheral blood of a subset of patients as measured by the percent change in mean tumor molecules per mL of blood; open circles represent non-detectable levels of ctDNA (left). Example of quantification of individual mutant ctDNA molecules detected in a no PFS-9 patient (blue) and a PFS-9 patient (green).

FIG. 3A-3F: Correlates with clinical response are observed pre-treatment in the tumor microenvironment (TME), including T cell infiltration, MHC Class II expression, and TCR diversity.

FIG. 3A. Correlation of CD4+(top) and CD8+(bottom) T cells per mm² either outside the tumor (left) or inside the tumor (right) in patient tumor biopsy material by multiplex IHC at the pre-treatment timepoint with PFS in months. Pearson's correlation coefficient (R) and the associated p value are indicated.

FIG. 3B. Representative images of IHC analysis of CD4+ and CD8+ T cells in patient tumor biopsy material are shown for one patient with no PFS-9 (2L3) and one patient with PFS-9 (2L5) stained with DAPI (blue), CD3 (red), CD4 (green), CD8 (white), and PanCK (cyan). White arrows indicate CD8+ T cells around the tumor area, and yellow arrows indicate CD4+ T cells infiltrating into the TME. (Scale bars, 50 μM).

FIG. 3C. Correlation of HLA Class II gene expression in the tumor biopsy at the pre-treatment timepoint with PFS in months. Pearson's correlation coefficient (R) and the associated p value are indicated.

FIG. 3D. Representative images of MHC Class II expression by multiplex IHC in patient tumor biopsy material are shown for one patient with no PFS-9 (2L3) and one patient with PFS-9 (2L5) stained with HLA-DR/DP/DQ (red), PanCK (green), DAPI (blue), CD11c (white) and CD14 (yellow). Individual channel images for patient 2L5 are shown below multiplex images for HLA-DR/DP/DQ, CD14, CD11c and PanCK. White arrows denote HLA-DR/DP/DQ+CD11c+CD14+ cells, and yellow arrow denote HLA-DR/DP/DQ+CD11c−CD14+ cells (Scale bars, 50 μm).

FIG. 3E. Analysis of Shannon's Entropy (left) and unique amino acid (UniAA) count (right) in the tumor biopsy at the pre-treatment timepoint and correlation with PFS in months. Pearson's correlation coefficient (R) and the associated p value are indicated.

FIG. 3F. Analysis of Shannon's Entropy (left) and unique amino acid count (right) at the pre-treatment (week 0), pre-vaccine (week 12) and post-vaccine (week 24) timepoints in tumor biopsy material are shown, with PFS-9 patients represented in green, and no PFS-9 patients represented in blue.

FIG. 4A-4E: NEO-PV-01 plus chemotherapy and anti-PD-1 induces neoantigen-reactive T cell responses that are neo-epitope specific, persistent, and show cytotoxic potential.

FIG. 4A. Percentage of all NEO-PV-01 vaccinating peptides that elicited IFNγ responses in serial PBMCs at the indicated timepoints.

FIG. 4B. Table summarizing overall immune responses detected for the 13 patients analyzed, also characterized as percent CD4+ or CD8+ responses.

FIG. 4C. Specificity of immune responses as measured by IFNγ ELISpot assay to mutant peptides (green) versus wild-type peptides (red) across a range of peptide concentrations. Representative responses from patient 2L7 (IM13 and IM04) and 2L3 (IM03 and IM15) are shown.

FIG. 4D. Persistence of immune responses induced by IM peptides, as measured by IFNγ ELISpot assay in PBMCs collected at week 52 after initiation of chemotherapy plus anti-PD-1 therapy. The data are represented as stacked columns for individual patients, with responses detected at both weeks 20 and 52 shown in light green, and responses detected only at week 20 shown in dark green.

FIG. 4E. Cytotoxic potential of NEO-PV-01-generated immune response as measured by the surface expression of the marker CD107a in combination with intracellular IFNγ expression at the post-vaccine timepoint. Representative flow plots for patient 2L7 are shown on the left, comparing peptide recall (bottom) with DMSO recall (top) in both the ex vivo (left) and 5-day stimulation (right) assay design. The data are summarized on the right, and represented as stacked columns for individual patients, with positive (cytotoxic potential) responses shown in dark green, and negative (no cytotoxic potential) responses shown in light green. The table below summarizes the responses detected for all patients analyzed, and categorizes responses as either CD4+, CD8+ or both. Aggregate data are represented as mean+/−SEM.

FIG. 5A-5E: NEO-PV-01 plus chemotherapy and anti-PD-1 induces epitope spread responses in the majority of patients analyzed, with mutKRAS responses observed.

FIG. 5A. Epitope spread was measured in 13 patients. Reactivity of the post-vaccine PBMCs against a range of 10-25 predicted neoantigen peptides that were not included in the vaccine were tested by IFNγ ELISpot assay. Responses characterized as epitope spread were detected only at the post-vaccine timepoint (week 20) and were not detected at the pre-vaccine time point (week 10).

FIG. 5B. T cell responses to individual non-immunizing (NIM) peptides across 9 patients at the post-vaccination timepoint are shown. NIM peptides that did not elicit reactivity post-vaccination are not shown. Each NIM peptide was tested for the generation of an immune response utilizing overlapping assay peptides, and the assay peptide that generated the maximum response in either the ex vivo assay or 5-day assay is shown. Responses with * denote ex vivo responses.

FIG. 5C. Four patients elicited mutKRAS-specific epitope spread responses as determined by IFNγ ELISpot assay. PFS-9 status of each patient is denoted below the graph, and specific G12 mutation denoted above the graph. Responses with * denote ex vivo responses.

FIG. 5D. Specificity of immune responses as measured by IFNγ ELISpot assay to mutant peptides (solid lines) versus wild-type peptides (dotted lines) across a range of peptide concentrations for each of the 4 patients where an epitope spread response to mutKRAS was observed.

FIG. 5E. Surface expression of the cytolytic marker CD107a (x axis) and intracellular expression of IFNγ (y axis) is shown by FACS analysis for the mutKRAS-specific epitope spread response observed for patient 2L15. Individual plots depict control (DMSO) on the left and NIM peptide on the right. Positivity in this assay was determined as a >1.5-fold stimulation over DMSO control in the double positive gate. Parent gates are indicated below the pair of FACS plots.

FIG. 6A-6H: Neoantigen-specific CD4+ T cell responses share an activated effector phenotype in the periphery following vaccination.

FIG. 6A. Table summarizing the 5 patients analyzed using a combination of multimer-based sorting of neoantigen-specific CD4+ T cells (a representative flow panel on right), CITE-Seq, TCRSeq and gene expression analysis.

FIG. 6B Unsupervised clustering (left) and heatmap (right) of normalized gene expression used for clustering analysis of tetramer+ and tetramer− CD4+ T cell samples from the 5 patients.

FIG. 6C. Unsupervised clustering plots separated to visualize tetramer− cells (top) and tetramer+ cells (bottom) from the 5 patients.

FIG. 6D. Proportions of each UMAP cluster comparing tetramer+ and tetramer− populations.

FIG. 6E. Measurement of the Gini coefficient (as an indicator of TCR repertoire clonality) for both tetramer+ and tetramer− populations. Each dot represents an individual patient.

FIG. 6F. Measurement of clonotypes covering the top 30% of the TCR repertoire of tetramer+ CD4+ T cells that were detected by single-cell TCR sequencing utilizing bulk TCR-seq data across the pre-treatment, pre-vaccine, and post-vaccine timepoints.

FIG. 6G. Measurement of the number of CD3+CD4+ T cells per mm² of tumor biopsy tissue using multiplex IHC at the pre-treatment, pre-vaccine and post-vaccine timepoints when available. Aggregate data are represented as mean+/−SEM.

FIG. 6H. Representative IHC images for patient 2L7 (PFS-9) at the pre-treatment (left), pre-vaccine (middle) and post-vaccine (right) timepoints are shown, stained with DAPI (blue), CD3 (red), CD4 (yellow), and PanCK (green). (Scale bars, 50 μm). Longitudinal biopsies were from the same lung lesion for this patient.

FIG. 7A-7C: Radiographic responses with pre-treatment tumor PD-L1 levels in vaccinated patients and tracking of individual ctDNA molecules longitudinally for each patient measuring abundance of predicted neoantigen genes and genes included in individualized Signatera pools.

FIG. 7A Radiographic responses with pre-treatment PD-L1 levels in tumors of patients who received at least one dose of NEO-PV-01. PD-L1 levels are indicated below the bars in the waterfall plots. Scoring was based on percent PD-L1 on tumor cells as follows: <1% is indicated as −, 1-<50% as + and ≥50% as ++.

FIG. 7B. Serial ctDNA measurements across 17 patients for 16 variants predicted to be high quality neoepitopes based on internal bioinformatics algorithms.

FIG. 7C. 16 selected somatic target mutations chosen by Natera's variant calling method.

FIG. 8A-SB: NEO-PV-01 plus chemotherapy and anti-PD-1 induces durable T cell reactivity against multiple vaccine neoepitopes.

FIG. 8A. T cell responses to individual immunizing (IM) peptides across 9 patients are shown. Immunizing peptides that did not elicit reactivity are not shown. Each IM peptide was tested for the generation of an immune response utilizing overlapping assay peptides, and the assay peptide that generated the maximum response for each IM peptide is shown across the pre-vaccine and post-vaccine timepoints. Each bar corresponds to the IM peptide and corresponding assay peptide that generates the maximal response in either the ex vivo assay or 5-day assay. Immunizing peptides labelled in red on the x axis elicited pre-vaccine responses.

FIG. 8B. T cell responses were quantified as in panel A, with additional analysis performed at 52 weeks after initiation of chemotherapy plus pembrolizumab for 4 patients. Inset for patient 2L11 is zoomed in to allow visualization of response to IM17 and IM07. Immunizing peptides labelled in red on the x axis elicited pre-vaccine responses, and those labelled in green elicited responses detected only at the week 52 timepoint. Aggregate data are represented as mean+/−SEM.

FIG. 9A-9B: NEO-PV-01 plus chemotherapy and anti-PD-1 induces cytotoxic CD4+ and CD8+ T cell responses. Surface expression of the cytolytic marker CD107a (x axis) and intracellular expression of IFNγ (y axis) is shown by FACS analysis for 56 IM peptides across 12 patients. Individual plots depict control (DMSO) on the left and IM peptide on the right. Only IM peptides that were positive in this assay (>1.5-fold stimulation over DMSO control in the double positive gate) are shown. Parent gates are indicated below each pair of FACS plots, including responses categorized as either CD4+ T cell responses (FIG. 9A) or CD8+ T cell responses (FIG. 9B).

FIG. 10A-10E: NEO-PV-01 plus chemotherapy and anti-PD-1 induces epitope spread responses that are cytotoxic and persistent.

FIG. 10A. Cytotoxic potential of epitope spread responses as measured by the surface expression of the marker CD107a in combination with intracellular IFNγ expression at the post-vaccine timepoint. The data are represented as stacked columns for individual patients, with positive (cytotoxic potential) responses shown in dark green, and negative (no cytotoxic potential) responses shown in light green. The table below summarizes the responses detected for all patients analyzed, and categorizes responses as either CD4+, CD8+ or both.

FIG. 10B. Individual plots depict control (DMSO) on the left and non-immunizing (NIM) peptide on the right. Only NIM peptides that were positive in this assay (>1.5-fold stimulation over DMSO control in the double positive gate) are shown. Parent gates are indicated below each pair of FACS plots.

FIG. 10C. Distribution of PFS in months for patients who either did or did not harbor a KRAS mutation in the pre-treatment tumor biopsy (ITT set). Patients who were vaccinated are shown as “+” and unvaccinated patients are shown as “ ̌#”. The four patients where epitope spread responses to mutKRAS were observed are shown in green. All other patients are shown in black. Boxplots indicate 25%, 50% and 75% percentiles, and whiskers extend to the 95% confidence interval values. p values are derived from a two-tailed Student's t-test.

FIG. 10D. T cell responses to individual non-immunizing (NIM) peptides at the pre-vaccine (week 10), post-vaccine (week 20) and week 52 timepoint are shown across 3 patients. NIM peptides that did not elicit reactivity are not shown. Each NIM peptide was tested for the generation of an immune response utilizing overlapping assay peptides, and the assay peptide that generated the maximum response for each NIM peptide is shown across the three timepoints. Each bar corresponds to the NIM peptide and corresponding assay peptide that generates the maximal response in either the ex vivo assay or 5-day assay. NIM peptides labelled in red on the x axis elicited pre-vaccine responses, and those in green elicited responses only detected at the week 52 timepoint. Inset for patient 2L16 is zoomed in to allow visualization of response to NIM101 and NIM12.

FIG. 10E. Persistence of immune responses induced by NIM peptides, as measured by IFNγ ELISpot assay in PBMCs collected at week 52 after initiation of chemotherapy plus anti-PD-1 therapy. The data are represented as stacked columns for individual patients, with responses detected at both weeks 20 and 52 shown in light green, and responses detected only at week 20 shown in dark green. Aggregate data are represented as mean+/−SEM.

FIG. 11A-11G: Neoantigen-reactive CD4+ effector cell phenotypes are distinct from other effector cells.

FIG. 11A. Table summarizing the 5 patients analyzed using a combination of multimer sorting of neoantigen-specific CD4+ T cells with corresponding mutant and wild-type peptide sequences shown for the response analyzed. NA=not applicable. Figure discloses SEQ ID NOS 44-52, respectively, in order of columns.

FIG. 11B. Comparison of antibody (1^{st}, 3^{rd} and 5^{th} rows) and RNA (2^{nd}, 4^{th} and 6^{th} rows) expression levels of genes that were included in the CITE antibody panel.

FIG. 11C. Unsupervised clustering of tetramer− and tetramer+ samples with normalized expression of selected RNA markers used for clustering.

FIG. 11D. Effector cells (described as CD45RO+CD45RA−CD62L^{lo}CCR7^{hi}) identified in blue among the unsupervised clustering of tetramer+ and tetramer− CD4+ T cell samples. All other cells are shown in pink.

FIG. 11E. (Top) Unsupervised clustering of only effector cells (both tetramer+ and tetramer−) results in 5 unique clusters. (Bottom) Unsupervised clustering plots separated to visualize tetramer− cells and tetramer+ cells among all effector cells.

FIG. 11F. Unsupervised clustering of the effector cells split by individual patient.

FIG. 11G. Heatmap of normalized gene expression for each of the 5 unique clusters identified based on clustering of all effector cells.

FIG. 12: Neoantigen-specific CD4+ T cell responses were detected by MHC Class II tetramer staining and exhibited effector and central memory phenotypes by flow cytometry. MHC Class II tetramer analysis was performed on CD4+ T cells from 8 patients across 27 peptide-MHC combinations at the post-vaccination timepoint. The leftmost plots for each patient depict the tetramer+ population in green circles (quantified both by total tetramer+ cell number and percent of the bulk CD4+ population). For a subset of patients, additional phenotyping was performed by flow cytometry to characterize the cells as naïve, effector, effector memory, or central memory based on CD45RA and CD62L expression (rightmost plots).

FIG. 13A-13I: Neoantigen-specific CD4+ T cells show distinct phenotypes across patients, but consistent phenotypes when comparing across clones targeting an individual epitope, and neoantigen-reactive TCRs are functional post-vaccination and tumors show accumulation of peripherally expanded TCRs.

FIG. 13A. Table outlining the TCR clones covering the top 30% of the repertoire per patient from the single cell TCRseq data and the corresponding frequency of tetramer+ CD4+ T cell clones for that patient in the peripheral blood at the post-vaccination timepoint.

FIG. 13B. Unsupervised clustering of only the tetramer+ samples with corresponding gene and protein expression markers specific for each cluster.

FIG. 13C. Unsupervised clustering of tetramer+ samples depicting normalized expression of selected genes used for clustering.

FIG. 13D. Heatmap of normalized gene expression based on clustering of only the tetramer+ samples.

FIG. 13E. Unsupervised clustering of re-clustered tetramer+ samples, split by individual patient.

FIG. 13F. Heatmap representing the abundance of cells in each clonotype across clusters of tetramer+ CD4+ T cells. Top expanded clones are defined as those covering the top 30% of the TCR repertoire for each patient detected by single-cell TCR-Seq.

FIG. 13G. CD4+ TCR clones identified by single cell TCRseq to specifically recognize a corresponding immunizing peptide epitope in the context of a matched MHC Class II allele were cloned into the Jurkat cell line (performed separately for each patient shown). Each of the 4 clonal Jurkat cell lines was co-cultured with matched patient APCs plus the corresponding IM peptide at various peptide concentrations, and secretion of IL-2 was measured in the supernatant as a readout for recognition of the peptide:MHC complex by the TCR. Reactivity to the corresponding wild-type sequence was tested as well, with the exception of patient 2L11/IM18 in which the mutation is a frameshift, where no wild-type peptide is available.

FIG. 13H. Measurement of the number of CD3+CD8+ T cells per mm² of tumor biopsy tissue using multiplex IHC at the pre-treatment, pre-vaccine and post-vaccine timepoints when available. Aggregate data are represented as mean+/−SEM.

FIG. 13I. TCR sequencing was performed on select post-vaccination tumor biopsy samples where corresponding pre-treatment or pre-vaccine biopsy material was also available. TCRs found exclusively in the post-vaccination biopsy were then cross-referenced with TCRs found in the peripheral blood. Only TCRs that were found to increase at the post-vaccination timepoint in the peripheral blood are visualized. TCRs that expanded upon vaccination in the periphery are shown in red, and TCRs that were detected only at the post-vaccination timepoint in the periphery are shown in blue.

### DETAILED DESCRIPTION

Described herein are new immunotherapeutic agents and uses thereof based on the discovery of neoantigens arising from mutational events unique to an individual's tumor. Accordingly, the present disclosure described herein provides peptides, polynucleotides encoding the peptides, and peptide binding agents that can be used, for example, to stimulate an immune response to a tumor associated antigen or neoepitope, to create an immunogenic composition or cancer vaccine for use in treating disease.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. Definitions

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In some embodiments of this application, the use of “or” means “and/or” unless stated otherwise. The terms “and/or” and “any combination thereof” and their grammatical equivalents as used herein, can be used interchangeably. In some embodiments, these terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

In some embodiments the term “about” or “approximately” can mean within an acceptable error range for the particular value as determined by one of ordinary skill in the art, which will depend in part on how the value is measured or determined, i.e., the limitations of the measurement system. For example, in some embodiments “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

“Major Histocompatibility Complex” or “MHC” in some embodiments can be a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, Fundamental Immunology, 3^{rd} Ed., Raven Press, New York (1993). “Proteins or molecules of the major histocompatibility complex (MHC)”, “MHC molecules”, “MHC proteins” or “HLA proteins” can be understood as meaning proteins capable of binding peptides resulting from the proteolytic cleavage of protein antigens and representing potential lymphocyte epitopes, (e.g., T cell epitope and B cell epitope) transporting them to the cell surface and presenting them there to specific cells, in particular cytotoxic T-lymphocytes, T-helper cells, or B cells. The major histocompatibility complex in the genome comprises the genetic region whose gene products can be expressed on the cell surface are important for binding and presenting endogenous and/or foreign antigens and thus for regulating immunological processes. The major histocompatibility complex can be classified into two gene groups coding for different proteins, namely molecules of MHC class I and molecules of MHC class II. The cellular biology and the expression patterns of the two MHC classes are believed to be adapted to these different roles.

“Human Leukocyte Antigen” or “HLA” in some embodiments can be a human class I or class II Major Histocompatibility Complex (MHC) protein (see, e.g., Stites, et al., Immunology, 8^{th} Ed., Lange Publishing, Los Altos, Calif. (1994).

“Polypeptide”, “peptide” and their grammatical equivalents as used herein can refer to, in some embodiments a polymer of amino acid residues. A “mature protein” can be a protein which is full-length and which, optionally, includes glycosylation or other modifications typical for the protein in a given cellular environment. Polypeptides and proteins disclosed herein (including functional portions and functional variants thereof) can comprise synthetic amino acids in place of one or more naturally-occurring amino acids. Such synthetic amino acids can be known in the art, and can include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, omithine, α-aminocyclopentane carboxylic acid, α-aminocyclohexane carboxylic acid, α-aminocycloheptane carboxylic acid, α-(2-amino-2-norbomane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications can include phosphorylation, acylation including acetylation and formylation, glycosylation (including N-linked and O-linked), amidation, hydroxylation, alkylation including methylation and ethylation, ubiquitination, addition of pyrrolidone carboxylic acid, formation of disulfide bridges, sulfation, myristoylation, palmitoylation, isoprenylation, famesylation, geranylation, glypiation, lipoylation and iodination. The term “polypeptide” or “peptide” can also mean that a polypeptide that has been separated from components that naturally accompany it. Typically, the polypeptide can be isolated when it is at least 60%, by weight, free from the proteins and naturally-occurring organic molecules with which it is naturally associated. In some embodiments, the preparation can be at least 75%, at least 90%, or at least 99%, by weight, a polypeptide. An isolated polypeptide can be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

In some embodiments an “immunogenic” peptide or an “immunogenic” epitope or “peptide epitope” can be a peptide that comprises an allele-specific motif such that the peptide will bind an HLA molecule and induce a cell-mediated or humoral response, for example, cytotoxic T lymphocyte (CTL (e.g., CD8⁺)), helper T lymphocyte (Th (e.g., CD4⁺)) and/or B lymphocyte response. Thus, immunogenic peptides described herein can be capable of binding to an appropriate HLA molecule and thereafter inducing a CTL (cytotoxic) response, or a HTL (and humoral) response, to the peptide.

In some embodiments the term “neoantigen” or “neoantigenic” can mean a class of tumor antigens that arises from a tumor-specific mutation(s) which alters the amino acid sequence of genome encoded proteins. Neoantigens encompass, but are not limited to, tumor antigens which arise from, for example, substitution in the protein sequence, frame shift mutation, fusion polypeptide, in-frame deletion, insertion, expression of endogenous retroviral polypeptides, and tumor-specific overexpression of polypeptides.

In some embodiments the terms “neoantigen peptide” and “neoantigenic peptide”, used interchangeably with “peptide” in the present specification, can refer to a series of residues, typically L-amino acids, connected one to the other, typically by peptide bonds between the α-amino and carboxyl groups of adjacent amino acids. Similarly, In some embodiments the term “polypeptide” can be used interchangeably with “mutant polypeptide”, “neoantigen polypeptide” and “neoantigenic polypeptide” in the present specification to designate a series of residues, e.g., L-amino acids, connected one to the other, typically by peptide bonds between the α-amino and carboxyl groups of adjacent amino acids. In some embodiments the polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. In some embodiments a peptide or polypeptide as used herein can comprise at least one flanking sequence. In some embodiments the term “flanking sequence” as used herein can refer to a fragment or region of the neoantigen peptide that is not a part of the neoepitope. In some embodiments the term “residue” can refer to an amino acid residue or amino acid mimetic residue incorporated into a peptide or protein by an amide bond or amide bond mimetic, or nucleic acid (DNA or RNA) that can encode the amino acid or amino acid mimetic.

In some embodiments “neoplasia” can mean any disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). Lymphoproliferative disorders are also considered to be proliferative diseases.

In some embodiments the term “neoplasia vaccine” can refer to a pooled sample of neoplasia/tumor specific neoantigens, for example at least two, at least three, at least four, at least five, or more neoantigenic peptides. In some embodiments a “vaccine” is to be understood as meaning a composition for generating immunity for the prophylaxis and/or treatment of diseases (e.g., neoplasia/tumor). Accordingly, vaccines can be medicaments which comprise antigens and are intended to be used in humans or animals for generating specific defense and protective substance by vaccination. In some embodiments a “vaccine composition” or a “neoplasia vaccine composition” can include a pharmaceutically acceptable excipient, carrier or diluent.

In some embodiments immune checkpoints can affect inhibitory pathways that slow down or stop immune reactions and prevent excessive tissue damage from uncontrolled activity of immune cells. By “Checkpoint inhibitor” can refer to any small molecule chemical compound, antibody, nucleic acid molecule, or polypeptide, or fragments thereof, which can inhibit the inhibitory pathways, allowing more extensive immune activity. In certain embodiments, the checkpoint inhibitor is an inhibitor of the programmed death-1 (PD-1) pathway, for example an anti-PD1 antibody, such as, but not limited to Nivolumab. In other embodiments, the checkpoint inhibitor is an anti-cytotoxic T-lymphocyte-associated antigen (CTLA-4) antibody. In additional embodiments, the checkpoint inhibitor is targeted at another member of the CD28 CTLA4 (g superfamily such as BTLA, LAG3, ICOS, PDL1 or KIR Page et al., Annual Review of Medicine 65:27 (2014)). In some cases targeting a checkpoint inhibitor is accomplished with an inhibitory antibody or similar molecule. In other cases, it is accomplished with an agonist for the target.

In further additional embodiments, the inhibitor is targeted at a member of the TNF Superfamily such as CD40, OX40, CD 137, GITR, CD27 or TIM-3. In some cases targeting a member of the TNF Superfamily is accomplished with an inhibitory antibody or similar molecule. In other cases, it can be accomplished with an agonist for the target; examples of this class include the stimulatory targets CD40, OX40 and GITR.

The term “combination” can in some embodiments embrace the administration of a vaccine or immunogenic composition (e.g., a pooled sample of neoplasia/tumor specific neo antigens) and one or more inhibitors, such as a checkpoint inhibitor or chemotherapeutic agent, as part of a treatment regimen intended to provide a beneficial (additive or synergistic) effect from the co-action of one or more of these therapeutic agents. The combination can also include one or more additional agents, for example, but not limited to, chemotherapeutic agents, anti-angiogenesis agents and agents that reduce immune-suppression. The beneficial effect of the combination includes, but is not limited to, pharmacokinetic or pharmacodynamic co-action resulting from the combination of therapeutic agents. Administration of these therapeutic agents in combination typically can be carried out over a defined time period (for example, minutes, hours, days, or weeks depending upon the combination selected).

“Combination therapy” can be intended to embrace administration of these therapeutic agents in a sequential manner, that is, wherein each therapeutic agent can be administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present disclosure can comprise a pooled sample of tumor specific neoantigens and an inhibitor, such as a checkpoint inhibitor or chemotherapeutic agent, administered at the same or different times, or the composition can be formulated as a single, co-formulated pharmaceutical composition comprising the two compounds. As another example, a combination of the present disclosure (e.g., a pooled sample of tumor specific neoantigens and an inhibitor, such as a checkpoint inhibitor (e.g., an anti-PD-L1 antibody), and/or a chemotherapeutic agent) can be formulated as separate pharmaceutical compositions that can be administered at the same or different time. As used herein, the term “simultaneously” can refer to administration of one or more agents at the same time. For example, in certain embodiments, a vaccine or immunogenic composition and an inhibitor, such as a checkpoint inhibitor or chemotherapeutic agent, are administered simultaneously. Simultaneously includes administration contemporaneously, that is during the same period of time. In certain embodiments, the one or more agents can be administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination can be administered by intravenous injection while the other component(s) of the combination can be administered orally. The components can be administered in any therapeutically effective sequence. The phrase “combination” embraces groups of compounds or non-drug therapies useful as part of a combination therapy.

The term “pharmaceutically acceptable” can refer to approved or approvable by a regulatory agency of the Federal or a state government or listed in the U.S. Pharmacopeia or other generally recognized pharmacopeia for use in animals, including humans. Examples of cancers can include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). Lymphoproliferative disorders are also considered to be proliferative diseases.

A “pharmaceutically acceptable excipient, carrier or diluent” can refer to an excipient, carrier or diluent that can be administered to a subject, together with an agent, and which can not destroy the pharmacological activity thereof and is nontoxic when administered in doses sufficient to deliver a therapeutic amount of the agent.

A “pharmaceutically acceptable salt” of pooled tumor specific neoantigens as recited herein can in some embodiments be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, in some embodiments the terms “prevent,” “preventing,” “prevention,” “prophylactic treatment,” and the like, can refer to reducing the probability of developing a disease or condition in a subject, who does not have, but is at risk of or susceptible to developing a disease or condition.

The term “prime/boost” or “prime/boost dosing regimen” can refer to the successive administrations of a vaccine or immunogenic or immunological compositions. The priming administration (priming) can be the administration of a first vaccine or immunogenic or immunological composition type and can comprise one, two or more administrations. The boost administration can be the second administration of a vaccine or immunogenic or immunological composition type and can comprise one, two or more administrations, and, for instance, can comprise or consist essentially of annual administrations. In certain embodiments, administration of the neoplasia vaccine or immunogenic composition can be in a prime/boost dosing regimen.

Ranges provided herein can be understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 can comprise 1 to 10, 1 to 20, 1 to 30, and 1 to 40 in one direction, or 50 to 40, 50 to 30, 50 to 20, and 50 to 10 in the other direction.

A “receptor” can be understood as meaning a biological molecule or a molecule grouping capable of binding a ligand. A receptor can serve, to transmit information in a cell, a cell formation or an organism. The receptor comprises at least one receptor unit and frequently contains two or more receptor units, where each receptor unit can consist of a protein molecule, in particular a glycoprotein molecule. The receptor can have a structure that complements the structure of a ligand and can complex the ligand as a binding partner. Signaling information can be transmitted by conformational changes of the receptor following binding with the ligand on the surface of a cell. According to the disclosure, a receptor can refer to particular proteins of MHC classes I and II capable of forming a receptor/ligand complex with a ligand, in particular a peptide or peptide fragment of suitable length.

The term “subject” refers to an animal which can be the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treat,” “treated,” “treating,” “treatment,” and the like can be meant to refer to reducing or ameliorating a disorder and/or symptoms associated therewith (e.g., a neoplasia or tumor). ‘Treating” can refer to administration of the combination therapy to a subject after the onset, or suspected onset, of a cancer. “Treating” can include the concepts of “alleviating”, which refers to lessening the frequency of occurrence or recurrence, or the severity, of any symptoms or other ill effects related to a cancer and/or the side effects associated with cancer therapy. The term “treating” can also encompass the concept of “managing” which refers to reducing the severity of a particular disease or disorder in a patient or delaying its recurrence, e.g., lengthening the period of remission in a patient who had suffered from the disease. It can be appreciated that, although not precluded, treating a disorder or condition does not require that the disorder, condition, or symptoms associated therewith be completely eliminated.

The term “therapeutic effect” can refer to some extent of relief of one or more of the symptoms of a disorder (e.g., a neoplasia or tumor) or its associated pathology. “Therapeutically effective amount” as used herein can refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. ‘Therapeutically effective amount” can be intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the disclosure employed in a pharmaceutical composition at levels lower than that required in order to achieve the desired therapeutic effect and gradually increase the dosage until the desired effect is achieved.

An “adverse reaction” or AE can generally refer to any untoward medical occurrence in a patient administered a pharmaceutical product, which can not necessarily have a causal relationship with the treatment. An AE can be any unfavorable and unintended sign (e.g., including an abnormal laboratory finding), symptom, or disease temporally associated with the use of the investigational product, whether or not it is considered to be study treatment related. This includes any newly occurring event or previous condition that has increased in severity or frequency since the administration of study treatment. Abnormal laboratory values or test results can constitute AEs only if they induce clinical signs or symptoms, are considered clinically significant, or require therapy.

Progression of the cancer under study cannot be considered an AE unless it is considered to be drug-related by the patients' care team. An “adverse drug reaction (ADR)” can be defined as all noxious and unintended responses to a medicinal product, related to any dose. A causal relationship between the medicinal product and an AE is at least a reasonable possibility—i.e., the relationship cannot be ruled out. An expected AE can be one that is listed or characterized in the applicable product information, e.g., the current IB. An unexpected AE can be one that is not identified in nature, severity, or frequency as described in the applicable product information, e.g., the current IB. An unexpected ADR can be an ADR where the nature or severity is not consistent with the applicable product information. ADRs that are more specific or more severe than described in the IB(s) can also be considered unexpected. A “serious adverse event” or SAE can be any AE, occurring at any dose and regardless of causality that: results in death; is life-threatening (Life-threatening means that the patient was at immediate risk of death from the reaction as it occurred, i.e., it cannot include a reaction which hypothetically might have caused death had it occurred in a more severe form); requires in-patient hospitalization or prolongation of existing hospitalization (hospitalization admissions and/or surgical operations scheduled to occur during the study period but planned prior to study entry can not be considered AEs if the illness or disease existed before the patient was enrolled in the study, provided that it did not deteriorate in an unexpected manner during the study (e.g., surgery performed earlier than planned)); results in persistent or significant disability/incapacity (disability can be defined as a substantial disruption of a person's ability to conduct normal life functions); can be a congenital anomaly/birth defect or is an important medical event, defined as an event that can not result in death, be life-threatening, or require hospitalization but can be considered an SAE when, based on appropriate medical judgment, it can jeopardize the patient or patients and can require medical or surgical intervention to prevent one of the outcomes listed in the definitions for SAEs. Examples of such medical events include allergic bronchospasm requiring intensive treatment in an emergency room or at home, blood dyscrasias or convulsions that do not result in in-patient hospitalization, or the development of drug dependency or drug abuse.

Each patient can be carefully monitored for the development of any AEs from the signing of consent through 30 days following the cessation of treatment. This information can be obtained in the form of non-leading questions (e.g., “How are you feeling?”) and from signs and symptoms detected during each examination, observations of study personnel, and spontaneous reports from patients. All AEs (serious and non-serious) spontaneously reported by the patient and/or in response to an open question from study personnel or revealed by observation, physical examination, or other diagnostic procedures can be recorded on the appropriate page of the eCRF. When possible, signs and symptoms indicating a common underlying pathology can be noted as one comprehensive event. All SAEs that occur from the signing of the ICF through 90 days after the last dose of nivolumab, or 30 days after the last dose of nivolumab if the patient initiates new anticancer therapy, should be reported by the Investigator to the Sponsor assigned to the study (see below) within 1 working day from the point in time when the Investigator becomes aware of the SAE. Should the Sponsor not be available for any reason, there can be an alternate physician contact. All SAEs can be reported whether or not considered causally related to the study treatment. SAE forms can be completed, and the information collected can include patient number, a narrative description of the event, and an assessment by the Investigator as to the severity of the event and relatedness to study treatment. A sample of the follow-up information on the SAE can be requested by the Sponsor or CRO.

The present disclosure relates to methods for the treatment of neoplasia, and more particularly tumors, by administering to a subject a neoplasia vaccine or immunogenic composition comprising a plurality of neoplasia/tumor specific neoantigens and at least one an inhibitor, such as a checkpoint inhibitor or chemotherapeutic agent.

Human tumors can contain large numbers of unique deoxyribonucleic acid (DNA) mutations that result in altered amino acid sequences of the encoded proteins. These novel protein sequences, often known as neoantigens, range from single amino acid changes (caused by missense mutations) to the addition of long regions of novel amino acid sequences due to frame shifts, read-through of termination codons, or translation of intron regions (novel open reading frames [neoORFs]). Tumor neoantigens arise mostly because of mutations in tumors. Therefore, they are extremely tumor-specific and are not subject to the immune-dampening effects of self-tolerance.

Immune responses to neoantigens can depend critically on the ability of major histocompatibility complex (MHC) molecules to effectively bind a small peptide (epitope) containing the altered amino acid sequence and present it to a T cell. Such an epitope can be generated synthetically and used in a vaccine to initiate an antigen-specific T-cell response targeting tumor cells expressing the mutated protein.

Binding of peptides to MHC can be used as a surrogate for the immunogenicity of a given peptide sequence. Advanced algorithms predicting peptide binding to MHC have been built using binding data from a large number of peptides to different MHC molecules (Lundegaard, 2011). These algorithms can be used to predict with high accuracy whether a specific peptide sequence will bind to MHC and with what affinity. Using these algorithms, protein sequences containing tumor-encoded mutations (both missense and neoORF) can be evaluated in silico for binding to a specific MHC molecule.

In some embodiments, a subject can comprise mutated epitopes comprising altered amino acid sequences, for example if the subject has cancer. In one embodiment mutated epitopes are determined by sequencing the genome and/or exome of tumor tissue and healthy tissue from a cancer patient using next generation sequencing technologies. In another embodiment genes that are selected based on their frequency of mutation and ability to act as a neoantigen are sequenced using next generation sequencing technology. Next-generation sequencing applies to genome sequencing, genome resequencing, transcriptome profiling (RNA-Seq), DNA-protein interactions (ChiP-sequencing), and epigenome characterization (de Magalhaes J P, Finch C E, Janssens G (2010). “Next-generation sequencing in aging research: emerging applications, problems, pitfalls and possible solutions”. Ageing Research Reviews 9 (3): 315-323; Hall N (Can 2007). “Advanced sequencing technologies and their wider impact in microbiology”. J. Exp. Biol. 209 (Pt 9): 1518-1525; Church G M (January 2006). “Genomes for ail”. Sci. Am. 294 (1): 46-54; ten Bosch J R, Grody W W (2008). “Keeping Up with the Next Generation”. The Journal of Molecular Diagnostics 10 (6): 484-492; Tucker T, Marra M, Friedman J M (2009). “Massively Parallel Sequencing: The Next Big Thing in Genetic Medicine”. The American Journal of Human Genetics 85 (2): 142-154).

Next-generation sequencing can now rapidly reveal the presence of discrete mutations such as coding mutations in individual tumors, most commonly single amino acid changes (e.g., missense mutations) and less frequently novel stretches of amino acids generated by frame-shift insertions/deletions/gene fusions, read-through mutations in stop codons, and translation of improperly spliced introns (e.g., neoORFs). NeoORFs are particularly valuable as immunogens because the entirety of their sequence is completely novel to the immune system and so are analogous to a viral or bacterial foreign antigen. Thus, neoORFs: (1) are highly specific to the tumor (i.e. there is no expression in any normal cells); (2) can bypass central tolerance, thereby increasing the precursor frequency of neoantigen-specific CTLs. For example, the power of utilizing analogous foreign sequences in a therapeutic anti-cancer vaccine or immunogenic composition was recently demonstrated with peptides derived from human papilloma virus (HPV). −50% of the 19 patients with pre-neoplastic, viral-induced disease who received 3-4 vaccinations of a mix of HPV peptides derived from the viral oncogenes E6 and E7 maintained a complete response for >24 months (Kenter et a, Vaccination against HPV-16 Oncoproteins for Vulvar Intraepithelial Neoplasia NEJM 361: 1838 (2009)).

Sequencing technology has revealed that each tumor contains multiple, patient-specific mutations that alter the protein coding content of a gene. Such mutations create altered proteins, ranging from single amino acid changes (caused by missense mutations) to addition of long regions of novel amino acid sequence due to frame shifts, read-through of termination codons or translation of intron regions (novel open reading frame mutations; neoORFs). These mutated proteins are valuable targets for the host's immune response to the tumor as, unlike native proteins; they are not subject to the immune-dampening effects of self-tolerance. Therefore, mutated proteins are more likely to be immunogenic and are also more specific for the tumor cells compared to normal cells of the patient.

An alternative method for identifying tumor specific neoantigens is direct protein sequencing. Protein sequencing of enzymatic digests using multidimensional MS techniques (MSn) including tandem mass spectrometry (MS/MS)) can also be used to identify neoantigens of the disclosure. Such proteomic approaches permit rapid, highly automated analysis (see, e.g., Gevaert and J. Vandekerckhove, Electrophoresis 21: 1145-1154 (2000)). It is further contemplated within the scope of the disclosure that high-throughput methods for de novo sequencing of unknown proteins can be used to analyze the proteome of a patient's tumor to identify expressed neoantigens. For example, meta shotgun protein sequencing can be used to identify expressed neoantigens (see e.g., Gutilais et al. (2012) Shotgun Protein Sequencing with Meta-contig Assembly, Molecular and Cellular Proteomics 11(30): 3084-96).

Tumor specific neoantigens can also be identified using MHC multimers to identify neoantigen-specific T-cell responses. For example, high-throughput analysis of neoantigen-specific T-cell responses in patient samples can be performed using MHC tetramer-based screening techniques (see e.g., Hombrink et al. (2011) High-Throughput Identification of Potential Minor Histocompatibility Antigens by MHC Tetramer-Based Screening: Feasibility and Limitations 6(8): 1-11; Hadrup et al. (2009) Parallel detection of antigen-specific T-cell. responses by multidimensional encoding of MHC multimers, Nature Methods, 6(7):520-26; van Rooij et al. (2013) Tumor exome analysis reveals neoantigen-specific T-cell reactivity in an Ipilimumab-responsive melanoma, Journal of Clinical Oncology, 31: 1-4; and Heemskerk et al. (2013) The cancer antigenome, EMBO Journal, 32(2):194-203). Such tetramer-based screening techniques can be used for the initial identification of tumor specific neoantigens, or alternatively as a secondary screening protocol to assess what neoantigens a patient can have already been exposed to, thereby facilitating the selection of candidate neoantigens for the disclosure.

In one embodiment the sequencing data derived from determining the presence of mutations in a cancer patient is analyzed to predict personal mutated peptides that can bind to HLA molecules of the individual. In one embodiment the data is analyzed using a computer. In another embodiment the sequence data is analyzed for the presence of neoantigens. In one embodiment neoantigens are determined by their affinity to MHC molecules. Efficiently choosing which particular mutations to utilize as immunogen requires identification of the patient HLA type and the ability to predict which mutated peptides would efficiently bind to the patient's HLA alleles. Recently, neural network based learning approaches with validated binding and non-binding peptides have advanced the accuracy of prediction algorithms for the major HLA-A and HLA-B alleles. Utilizing the recently improved algorithms for predicting which missense mutations create strong binding peptides to the patient's cognate MHC molecules, a set of peptides representative of optimal mutated epitopes (both neoORF and missense) for each patient can be identified and prioritized (Zhang et al, Machine learning competition in immunology—Prediction of HLA class I binding peptides J Immunol Methods 374: 1 (2011); Lundegaard et al Prediction of epitopes using neural network based methods J Immunol Methods 374:26 (2011)).

Targeting as many mutated epitopes as practically possible takes advantage of the enormous capacity of the immune system, prevents the opportunity for immunological escape by down-modulation of a particular immune targeted gene product, and compensates for the known inaccuracy of epitope prediction approaches. Synthetic peptides provide a particularly useful means to prepare multiple immunogens efficiently and to rapidly translate identification of mutant epitopes to an effective vaccine or immunogenic composition. Peptides can be readily synthesized chemically and easily purified utilizing reagents free of contaminating bacteria or animal substances. The small size allows a clear focus on the mutated region of the protein and also reduces irrelevant antigenic competition from other components (unmutated protein or viral vector antigens).

In one embodiment the drug formulation is a multi-epitope vaccine or immunogenic composition of long peptides. Such “long” peptides undergo efficient internalization, processing and cross-presentation in professional antigen-presenting cells such as dendritic cells, and have been shown to induce CTLs in humans (Melief and van der Burg, Immunotherapy of established (pre) malignant disease by synthetic long peptide vaccines Nature Rev Cancer 8:351 (2008)). In one embodiment at least 1 peptide is prepared for immunization. In some embodiments, 20 or more peptides are prepared for immunization. In one embodiment the neoantigenic peptide ranges from about 5 to about 50 amino acids in length. In another embodiment peptides from about 15 to about 35 amino acids in length is synthesized. In some embodiments, the neoantigenic peptide ranges from about 20 to about 35 amino acids in length.

In some embodiments, personalized cancer vaccines consisting of up to 20 synthesized peptides approximately 14 to 35 amino acids in length that are derived from an individual patient's mutated tumor DNA (neoantigens) are provided. Because these mutations are not expressed in the patient's normal cells, they are specific targets expressed only on tumor cells.

Unlike most previously used cancer vaccines, this neoantigen peptide vaccine is based on the production of a novel and unique product for each individual patient or cancer phenotype. The extent of possible tumor mutations and the wide range of patient human leukocyte antigen (HLA) haplotypes make it highly unlikely that any 2 patients will receive the same vaccine.

Generation of neoantigens can begin with whole exome DNA and ribonucleic acid (RNA) sequencing of tumor and normal tissue samples and HLA-A, HLA-B, and HLA-C genotypes from a subject. These data can then be used to identify coding sequence mutations that have occurred in the subject's tumor. These mutations can in some cases include single-amino acid missense mutations, fusion proteins, and neoORFs which can vary in length from 1 amino acid up to hundreds of amino acids. Long peptides 14-35 residues in length can then be designed specifically from the specific mutations identified in an individual's tumor. The vaccine can then be composed of a mixture of peptides that are predicted to induce a response in CD4+ and/or CD8+ T cells. In order to predict which are most likely to induce such an immune response, a number of filters can be applied to the entire set of long peptides that cover the subject's tumor mutanome. A primary criterion is the HLA binding affinity of the mutant epitope compared to its native protein. An epitope selection algorithm can be used to identify mutation-containing epitopes that are predicted to bind to MHC class I molecules of each subject (Lundegaard, 2011). Other key criteria can include RNA expression, type of mutation (e.g., missense versus neoORF), the likelihood that the mutation is an oncogenic driver, and the physical location of the mutant residue(s) on the peptide. Up to 35 peptides can be selected and prioritized for synthesis. Thereafter, up to 20 synthesized peptides can be mixed together in up to 4 pools of up to 5 peptides each for injection. Each of the 4 pools can be injected in to the subject.

### II. Production of Tumor Specific Neoantigens

The present disclosure is based, at least in part, on the ability to present the immune system of the patient with a pool of tumor specific neoantigens. One of skill in the art from this disclosure a d the knowledge in the art will appreciate that there are a variety of ways in which to produce such tumor specific neoantigens. In general, such tumor specific neoantigens can be produced either in vitro or in vivo. Tumor specific neoantigens can be produced in vitro as peptides or polypeptides, which can then be formulated into a personalized neoplasia vaccine or immunogenic composition and administered to a subject. As described in further detail herein, such in vitro production can occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, tumor specific neoantigens can be produced in vivo by introducing molecules (e.g., DNA, RNA, viral expression systems, and the like) that encode tumor specific neoantigens into a subject, whereupon the encoded tumor specific neoantigens are expressed. The methods of in vitro a d in vivo production of neoantigens is also further described herein as it relates to pharmaceutical compositions and methods of deliver of the combination therapy.

### A. In Vitro Peptide/Polypeptide Synthesis

Proteins or peptides can be made by any technique known to those of skill in the art, including the expression of proteins, polypeptides or peptides through standard molecular biological techniques, the isolation of proteins or peptides from natural sources, in vitro translation, or the chemical synthesis of proteins or peptides. The nucleotide and protein, polypeptide and peptide sequences corresponding to various genes have been previously disclosed, and can be found at computerized databases known to those of ordinary skill in the art. Exemplary database can be found in the National Center for Biotechnology Information, Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield R B: Solid phase peptide synthesis. I. The synthesis of a tetrapeptide. J. Am. Chem. Soc. 85:2149-54, 1963). In certain embodiments, neoantigenic peptides are prepared by (1) parallel solid-phase synthesis on multi-channel instruments using uniform synthesis and cleavage conditions; (2) purification over a P-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different-patients.

Alternatively, a nucleic acid (e.g., a polynucleotide) encoding a neoantigenic peptide of the disclosure can be used to produce the neoantigenic peptide in vitro. The polynucleotide can be, e.g., DNA, cDNA, PNA, CNA, RNA, either single- and/or double-stranded, or native or stabilized forms of polynucleotides, such as e.g., polynucleotides with a phosphorothiate backbone, or combinations thereof and it can or can not contain introns so long as it codes for the peptide. In one embodiment in vitro translation is used to produce the peptide. Many exemplary systems exist that one skilled in the art could utilize (e.g., Retic Lysate IVT Kit, Life Technologies, Waltham, MA).

An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression, if necessary, the DNA can be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the isolated polynucleotides, as well as host cells containing the expression vectors, are also contemplated. The neoantigenic peptides can be provided in the form of RNA or cDNA molecules encoding the desired neoantigenic peptides. One or more neoantigenic peptides of the disclosure can be encoded by a single expression vector.

The term “polynucleotide encoding a polypeptide” encompasses a polynucleotide which includes only coding sequences for the polypeptide as well as a polynucleotide which includes additional coding and/or non-coding sequences. Polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides can comprise the coding sequence for the tumor specific neoantigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In embodiments, the polynucleotides can comprise the coding sequence for the tumor specific neoantigenic peptide fused in the same reading frame to a marker sequence that allows, for example, for purification of the encoded polypeptide, which can then be incorporated into the personalized neoplasia vaccine or immunogenic composition. For example, the marker sequence can be a hexa-histidine tag (SEQ ID NO: 1) supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides can comprise the coding sequence for one or more of the tumor specific neoantigenic peptides fused in the same reading frame to create a single concatamerized neoantigenic peptide construct capable of producing multiple neoantigenic peptides.

In certain embodiments, isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neoantigenic peptide of the present disclosure, can be provided.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95%) identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, WI 53711). Bestfit uses the local homology algorithm of Smit and Waterman, Advances in Applied Mathematics 2:482-489 (1981), to find the best segment of homology between two sequences. When using Bestfit or any other sequence alignment program to determine whether a particular sequence is, for instance, 95%) identical to a reference sequence according to the present disclosure, the parameters are set such that the percentage of identity is calculated over the full length of the reference nucleotide sequence and that gaps in homology of up to 5% of the total number of nucleotides in the reference sequence are allowed.

The isolated tumor specific neoantigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al, Proc. Nat'l. Acad. Sci. USA 81:5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors can be used to amplify and express DNA encoding the tumor specific neoantigenic peptides. Recombinant expression vectors are replicable DNA constructs which have synthetic or cDNA-derived DNA fragments encoding a tumor specific neoantigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, operatively linked means contiguous and in the reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as Ml 3 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al, Cloning Vectors: A Laboratory Manual, Elsevier, Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23: 175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine (SEQ ID NO: 1), maltose binding domain, influenza coat sequence, glutathione-S-transferase, and the like can be attached to the j 1 protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

### B. In Vivo Peptide/Polypeptide Synthesis

The present disclosure also contemplates the use of nucleic acid molecules as vehicles for delivering neoantigenic peptides/polypeptides to the subject in need thereof, in vivo, in the form of, e.g., DNA/RNA vaccines (see, e.g., WO2012/159643, and WO2012/159754, hereby incorporated by reference in their entirety).

In one embodiment neoantigens can be administered to a patient in need thereof by use of a plasmid. These are plasmids which usually consist of a strong viral promoter to drive the in vivo transcription and translation of the gene (or complementary DNA) of interest (Mor, et al., (1995). The Journal of Immunology 155 (4): 2039-2046). Intron A can sometimes be included to improve mRNA stability and hence increase protein expression (Leitner et al, (1997). The Journal of Immunology 159 (12): 6112-61 19). Plasmids also include a strong polyadenylation/transcriptional termination signal, such as bovine growth hormone or rabbit beta-globulin polyadenylation sequences (Alarcon et al, (1999). Adv. Parasitol. Advances in Parasitology 42: 343-410; Robinson et al., (2000). Adv. Virus Res. Advances in Virus Research 55: 1-74; Bohm et al, (1996). Journal of Immunological Methods 193 (i): 29-40). Multicistronic vectors are sometimes constructed to express more than one immunogen, or to express an immunogen and an immunostimulatory protein (Lewis et al., (1999). Advances in Virus Research (Academic Press) 54: 129-88).

Because the plasmid is the “vehicle” from which the immunogen is expressed, optimizing vector design for maximal protein expression is essential (Lewis et al., (1999), Advances in Vims Research (Academic Press) 54: 129-88). One way of enhancing protein expression is by optimizing the codon usage of pathogenic mRNAs for eukaryotic cells. Another consideration is the choice of promoter. Such promoters can be the SV40 promoter or Rous Sarcoma Vims (RSV).

Plasmids can be introduced into animal tissues by a number of different methods. The two most popular approaches are injection of DNA in saline, using a standard hypodermic needle, and gene gun delivery, A schematic outline of the construction of a DNA vaccine plasmid and its subsequent delivery by these two methods into a host is illustrated at Scientific American (Weiner et al., (1999) Scientific American 281 (I): 34-41). Injection in saline is normally conducted intramuscularly (IM) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999), Adv, Parasitol. Advances in Parasitology 42: 343-410; Lewis et al., (1999), Advances in Virus Research (Academic Press) 54: 12.9-88).

Alternative delivery methods can include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Vims Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors.

The method of delivery determines the dose of DNA required to raise an effective immune response. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshia et al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In one embodiment, a neoplasia vaccine or immunogenic composition can include separate DNA plasmids encoding, for example, one or more neoantigenic peptides/polypeptides as identified in according to the disclosure. As discussed herein, the exact choice of expression vectors can depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection.

One or more neoantigenic peptides of the disclosure can be encoded and expressed in vivo using a viral based system (e.g., an adenovirus system, an adeno associated vims (AAV) vector, a poxvirus, or a lentivirus). In one embodiment, the neoplasia vaccine or immunogenic composition can include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (1PCAVD 001). J Infect Dis. 2013 Jan. 15; 207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. Patent application No. 20080254008, hereby incorporated by reference).

Among vectors that can be used in the practice of the disclosure, integration in the host genome of a cell is possible with retrovirus gene transfer methods, often resulting in long term expression of the inserted transgene. In some embodiments, the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors can be used in the practice of the disclosure). Moreover, lentiviral vectors are able to transduce or infect non-dividing cells and typically produce high viral titers. Selection of a retroviral gene transfer system can therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that can be used in the practice of the disclosure include those based upon murine leukemia virus (MuLV), gibbon ape leukemia virus (GaLV), simian immunodeficiency virus (SIV), human immunodeficiency virus (HIV), and combinations thereof (see, e.g., Buchscher et al., (1992) J. Virol. 66:2731-2739; Johann et al., (1992) J. Virol. 66: 1635-1640; Sommnerfelt et al., (1990) Virol. 176:58-59; Wilson et al, (1998) J. Virol. 63:2374-2378; Miller et al., (1991) J. Virol. 65:2220-2224; PCT/US94/05700). Zo et al. administered about 10 μl of a recombinant lentivirus having a titer of 1×10¹ transducing units (TU)/ml by an intrathecal catheter. These sort of dosages can be adapted or extrapolated to use of a retroviral or lentiviral vector in the present disclosure.

Also useful in the practice of the disclosure is a minimal non-primate lentiviral vector, such as a lentiviral vector based on the equine infectious anemia vims (EIAV) (see, e.g., Balagaan, (2006) J Gene Med; 8: 275-285, Published online 21 Nov. 2005 in Wiley InterScience (interscience.wiley.com). DOI: 1002/jgm.845). The vectors can have cytomegalovirus (CMV) promoter driving expression of the target gene. Accordingly, the disclosure contemplates amongst vector(s) useful in the practice of the disclosure: viral vectors, including retroviral vectors and lentiviral vectors.

Also useful in the practice of the disclosure is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

In an embodiment herein the delivery is via an adenovirus, which can be at a single booster dose containing at least 1×10⁵ particles (also referred to as particle units, pu) of adenoviral vector. In an embodiment herein, the dose can be at least about 1×10⁶ particles (for example, about 1×10⁶-1×10² particles), at least about 1×10⁷ particles, at least about 1×10⁸ particles (e.g., about 1×10⁸-1×10¹¹ particles or about 1×10⁸-1×10¹² particles), or at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, no more than about 1×10¹³ particles, no more than about 1×10¹² particles, no more than about 1×10¹¹ particles, or no more than about 1×10¹⁰ particles (e.g., no more than about 1×10⁹ articles). Thus, the dose can contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al, granted on Jun. 4, 2013; incorporated by reference herein, and the dosages at col 29, lines 36-58 thereof In an embodiment herein, the adenovirus is delivered via multiple doses.

In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: SynapsinI for all neurons, CaMKIIalpha for excitatory neurons, GAD67 or GAD65 or VGAT for GABAergic neurons, etc. Promoters used to drive RNA synthesis can include: Pol II I promoters such as U6 or HI, The use of a Pol II promoter and intronic cassettes can be used to express guide RNA (gRNA).

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV 1, AAV2, AAV5 or any combination thereof for targeting brain or neuronal cells; and one can select AAV4 for targeting cardiac tissue. AAV8 is useful for delivery to the liver. The above promoters and vectors can be used individually.

In an embodiment herein, the delivery is via an AAV. A therapeutically effective dosage for in vivo delivery of the AAV to a human is believed to be in the range of from about 20 to about 50 ml of saline solution containing from about 1×10¹⁰ to about 1×10⁵⁰ functional AAV/ml solution. The dosage can be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage can be about 1×10¹³ genomes AAV. Such concentrations can be delivered in from about 0.001 ml to about 100 ml, about 0.05 to about 50 ml, or about 10 to about 25 ml of a carrier solution. In some embodiments, AAV is used with a titer of about 2×10¹³ viral genomes/milliliter, and each of the striatal hemispheres of a mouse receives one 500 nanoliter injection. Other effective dosages can be readily established by one of ordinary skill in the art through routine trials establishing dose response curves. (See, e.g., U.S. Pat. No. 8,404,658 B2, hereby incorporated by reference in its entirety).

In another embodiment effectively activating a cellular immune response for a neoplasia vaccine or immunogenic composition can be achieved by expressing the relevant neoantigens in a vaccine or immunogenic composition in a non-pathogenic microorganism. Well-known examples of such microorganisms are Mycobacterium bovis BCG, Salmonella and Pseudomonas (See, U.S. Pat. No. 6,991,797, hereby incorporated by reference in its entirety).

In another embodiment a Poxvirus is used in the neoplasia vaccine or immunogenic composition. These include orthopoxvirus, avipox, vaccinia, MVA, NYVAC, canarypox, ALVAC, fowlpox, TROVAC, etc. (See e.g., Verardi et al., Hum Vaccin Immunother. 2012 July; 8 (7):961-70; and Moss, Vaccine. 2013: 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

In another embodiment the vaccinia virus is used in the neoplasia vaccine or immunogenic composition to express a neoantigen. (See e.g., Rolph et al., Recombinant viruses as vaccines and immunological tools. Curr Opin Immunol 9:517-524, 1997). The recombinant vaccinia virus is able to replicate within the cytoplasm of the infected host cell and the polypeptide of interest can therefore induce an immune response. Moreover, Poxviruses have been widely used as vaccine or immunogenic composition vectors because of their ability to target encoded antigens for processing by the major histocompatibility complex class I pathway by directly infecting immune cells, in particular antigen-presenting cells, but also due to their ability to self-adjuvant.

In another embodiment ALVAC is used as a vector in a neoplasia vaccine or immunogenic composition. ALVAC is a canarypox vims that can be modified to express foreign transgenes and has been used as a method for vaccination against both prokaryotic and eukaryotic antigens (Horig H, Lee D S, Conkright W, et al. Phase I clinical trial, of a recombinant canarypoxvirus (ALVAC) vaccine expressing human carcinoembryonic antigen and the B7.1 co-stimulatory molecule. Cancer Immunol Immunother 2000; 49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000; 6:2219-28; Musey L, Ding Y, Elizaga M, et al. HIV-1 vaccination administered intramuscularly can induce both, systemic and mucosal T cell immunity in HIV-1-uninfected individuals. J Immunol 2003; 171:1094-101; Paoletti E. Applications of pox virus vectors to vaccination: an update. Proc Natl Acad Sci USA 1996; 93:11349-53; U.S. Pat. No. 7,255,862). In a phase I clinical trial, an ALVAC virus expressing the tumor antigen CEA showed an excellent safety profile and resulted in increased CEA-specific T-cell responses in selected patients; objective clinical responses, however, were not observed (Marshall J L, Hawkins M J, Tsang K Y, et al. Phase I study in cancer patients of a replication-defective avipox recombinant, vaccine that expresses human carcinoembryonic antigen. J Clin Oncol 1999; 17:332-7).

In another embodiment a Modified Vaccinia Ankara (MVA) virus can be used as a viral vector for a neoantigen vaccine or immunogenic composition. MVA is a member of the Orthopoxvirus family and has been generated by about 570 serial passages on chicken embryo fibroblasts of the Ankara strain of Vaccinia virus (CVA) (for review see Mayr, A., et al., Infection 3, 6-14, 1975). As a consequence of these passages, the resulting MVA vims contains 3.1 kilobases less genomic information compared to CVA, and is highly host-cell restricted (Meyer, H. et al., J. Gen. Virol. 72, 103 1-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast, cancer and is currently in clinical trials. (Mandl et al., Cancer Immunol Immunother. January 2012; 61(1): 19-29). Methods to make and use recombinant MVA have been described (e.g., see U.S. Pat. Nos. 8,309,098 and 5,185,146 hereby incorporated in its entirety).

In another embodiment the modified Copenhagen strain of vaccinia virus, NYVAC and NYVAC variations are used as a vector (see U.S. Pat. No. 7,255,862; PCT WO 95/30018; U.S. Pat. Nos. 5,364,773 and 5,494,807, hereby incorporated by reference in its entirety).

In one embodiment recombinant viral particles of the vaccine or immunogenic composition are administered to patients in need thereof. Dosages of expressed neoantigen can range from a few to a few hundred micrograms, e.g., 5 to 500.mu.g. The vaccine or immunogenic composition can be administered in any suitable amount to achieve expression at these dosage levels. The viral particles can be administered to a patient in need thereof or transfected into cells in an amount of about at least 10^{3.5} pfu; thus, the viral particles can be administered to a patient in need thereof or infected or transfected into cells in at least about 10⁴ pfu to about 10⁶ pfu; however, a patient in need thereof can be administered at least about 10⁸ pfu such that an amount for administration can be at least about 10⁷ pfu to about 10⁹ pfu. Doses as to NYVAC are applicable as to ALVAC, MVA, MVA-BN, and avipoxes, such as canarypox and fowlpox.

### III. Vaccine or Immunogenic Composition Adjuvant

Toll-like receptors (TLRs) are important members of the family of pattern recognition receptors (PRRs) expressed by cells of the innate and adaptive immune systems. The TLRs recognize conserved motifs shared by many microorganisms, termed pathogen-associated molecular patterns (PAMPS). Different TLRs recognize distinct PAMPs, and TLR ligand binding leads to activation of inflammatory signaling cascades including the nuclear factor kappa light-chain of activated B cells (NF-κB) transcription factor and the type I interferons (IFNs). Toll-like receptor-mediated activation of APCs such as dendritic cells (DCs) results in increased expression of MHC and T-cell co-stimulatory molecules and can help facilitate initiation of a peptide-specific T-cell response.

Non-limiting examples of cancer vaccine adjuvants include TLR9 agonist 5′-C-phoshate-G-3′ (CpG) and the synthetic double-stranded ribonucleic acid (dsRNA) TLR3 ligand polyinosinic-polycytidylic acid-polylysine carboxymethylcellulose (adjuvant) (poly-ICLC) [Hiltonol®](poly-inosinic acid: poly-cytidilic acid). The CpG is a synthetic dinucleotide, and pICLC is a synthetic, dsRNA stabilized with poly-lysine and carboxymethylcellulose.

Poly-ICLC is a synthetic, dsRNA “host-targeted” therapeutic viral-mimic and PAMP with broad innate and adaptive immune adjuvant function. Poly-ICLC exerts its function through TLR3, melanoma differentiation-associate protein 5 (MDA5), and several nuclear and cytoplasmic enzyme systems (oligoadenylate synthetase, the dsRNA-dependent protein kinase R [PKR], retinoic acid-inducible gene 1 [RIG-1] helicase, and MDA5) that are involved in antiviral and anti-tumor host defenses.

Stimulation with poly-ICLC leads to DC and natural killer (NK) cell activation and production of a natural mix of type I IFNS, cytokines, and chemokines (Meylan, 2006). This adjuvant has been shown to induce local and systemic activation of immune cells in vivo, produce stimulatory chemokines and cytokines, and stimulate antigen presentation by DCs. In preclinical studies, poly-ICLC appears to be a potent TLR adjuvant due to its induction of pro-inflammatory cytokines, lack of stimulation of Interleukin-10 (IL-10), and maintenance of high levels of co-stimulatory molecules in DCs (Bogunovic, 2011). Furthermore, poly-ICLC was directly compared to CpG in non-human primates as an adjuvant for a protein vaccine consisting of human papillomavirus (HPV) 16 capsomers and was found to be much more effective in inducing HPV-specific T_{H}1 (T helper cell 1) immune responses (Stahl-Hennig, 2009).

Poly-ICLC can induce durable CD4+ and CD8+ responses in humans. Striking similarities were seen in the up-regulation of transcriptional and signal transduction pathways between patients vaccinated with poly-ICLC and in volunteers who had received the highly effective, replication-competent yellow fever vaccine (Okada, 2011). In a recent Phase 1 study, >90% of ovarian carcinoma patients immunized with poly-ICLC in combination with a NY-ESO-1 peptide vaccine showed induction of CD4+ and CD8+ T cells, as well as antibody responses to the peptide (Sabbatini, 2012). Without being bound by theory, these neoantigens are expected to bypass central thymic tolerance (thus allowing stronger anti-tumor T cell response), while reducing the potential for autoimmunity (e.g., by avoiding targeting of normal self-antigens). An effective immune response advantageously includes a strong adjuvant to activate the immune system (Speiser and Romero, Molecularly defined vaccines for cancer immunotherapy, and protective T cell immunity Seminars in Immunol 22: 144 (2010)). For example, Toll-like receptors (TLRs) have emerged as powerful sensors of microbial and viral pathogen “danger signals”, effectively inducing the innate immune system, and in turn, the adaptive immune system (Bhardwaj and Gnjatic, TLR AGONISTS: Are They Good Adjuvants?Cancer J. 16:382-391 (2010)). Among the TLR agonists, poly-ICLC (a synthetic double-stranded RNA mimic) is one of the most potent activators of myeloid-derived dendritic cells. In a human volunteer study, poly-ICLC has been shown to be safe and to induce a gene expression profile in peripheral blood cells comparable to that induced by one of the most potent live attenuated viral vaccines, the yellow fever vaccine YF-17D (Caskey et al, Synthetic double-stranded RNA induces innate immune responses similar to a live viral vaccine in humans J Exp Med 208:2357 (2011)). In some embodiments, Hiitonol®, a GMP preparation of poly-ICLC prepared by Oncovir, Inc. is utilized as the adjuvant. In other embodiments, other adjuvants described herein are envisioned. For instance oil-in-water, water-in-oil or multiphasic W/O/W; see, e.g., U.S. Pat. No. 7,608,279 and Aucouturier et al, Vaccine 19 (2001), 2666-2672, and documents cited therein.

### IV. Immune Checkpoint Modulators

Immune checkpoints are crucial signaling pathways in the immune system that maintain self-tolerance and modulate the duration and amplitude of physiological immune responses. Under normal conditions, these pathways prevent excessive effector activity by T cells. Two important examples of this pathway are the cell surface receptors CTLA-4 and PD-1 (Teft, 2006; Keir, 2008). In some cases, tumors express or over-express inhibitory immune checkpoint pathways as a major mechanism of immune evasion. Because many of the immune checkpoints are initiated by ligand-receptor interactions, these signals can be readily blocked by antibodies or modulated by recombinant forms of ligands or receptors (Pardoll, 2012).

Immune checkpoint proteins are important targets for pharmacologic blockade (Teft, 2006; Keir, 2008), and dramatic clinical responses have been observed after treatment with antibodies blocking PD-1 and CTLA-4 (See, e.g., Brahmer, 2010; Robert, 2011; Topalian, 2012; Powles, 2014; Topalian, 2014; Brahmer, 2015; Le, 2015; Robert, 2015; Reck, 2016; Langer, 2017). Accordingly, the present disclosure features in exemplary embodiments, novel combinations of a neoplasia vaccine or immunogenic composition and an anti-PD-1 antibodies.

The PD-1 receptor refers to an immunoinhibitory receptor belonging to the CD28 family. PD-1 is expressed on a number of cell types including Tregs, activated B cells, and natural killer (NK) cells, and is expressed predominantly on previously activated T cells in vivo, and binds to two ligands, PD-L1 and PD-L2. PD1's endogenous ligands, PD-L1 and PD-L2, are expressed in activated immune cells as well as nonhematopoietic cells, including tumor cells. PD-1 as used herein is meant to include human PD-1 (hPD-1), variants, isoforms, and species homologs of hPD-1, and analogs having at least one common epitope with hPD-1. The complete hPD-1 sequence can be found under GENBANK Accession No. U64863. Programmed Death Ligand-1 (PD-L1 “is one of two cell surface glycoprotein ligands for PD-1 (the other being PD-L2) that downregulate T cell activation and cytokine secretion upon binding to PD-1. PD-L3 as used herein includes human PD-L1 (hPD-L1), variants, isoforms, and species homologs of hPD-L1, and analogs having at least one common epitope with hPD-L1. The complete hPD-L1 sequence can be found under GENBAN Accession No. Q9NZQ7. Tumors have been demonstrated to escape immune surveillance by expressing PD-L1/L2, thereby suppressing tumor-infiltrating lymphocytes via PD-1/PD-L1,2 interactions (Dong et al. Nat. Med. 8:793-800. 2002).

In some embodiments, the anti-PD-1 antibody is nivolumab. Nivolumab (Opdivo®, Bristol-Myers Squibb Company, NY) is a human immunoglobulin G4 (IgG4) mAb that binds to the programmed death 1 (PD-1) receptor and blocks its interaction with PD-L1 and programmed death ligand 2 (PD-L2), reversing PD-1 pathway-mediated inhibition of the immune response, including the anti-tumor immune response. Binding of PD-L1 and PD-L2 to the PD-1 receptor expressed on T cells inhibits T-cell proliferation and cytokine production. Up-regulation of PD-1 ligands occurs in some tumors, and signaling through this pathway can contribute to inhibition of active T-cell immune surveillance of tumors. The antibodies of the disclosure include, but are not limited to, all of the anti-PD-1 and anti-PD-L1 Abs disclosed in U.S. Pat. Nos. 8,008,449 and 7,943,743, respectively. Other anti-PD-1 mAbs have been described in, for example, U.S. Pat. Nos. 7,488,802 and 8,168,757, and anti-PD-L1 mAbs have been described in, for example, U.S. Pat. Nos. 7,635,757 and 8,217,149, and U.S. Publication No. 2009/0317368. U.S. Pat. No. 8,008,449 exemplifies seven anti-PD-1 HuMAbs: 1 708. 2D3, 4M, 5C4 (also referred to herein as nivolumab or BMS-936558), 4A11, 7D3 and 5F4.

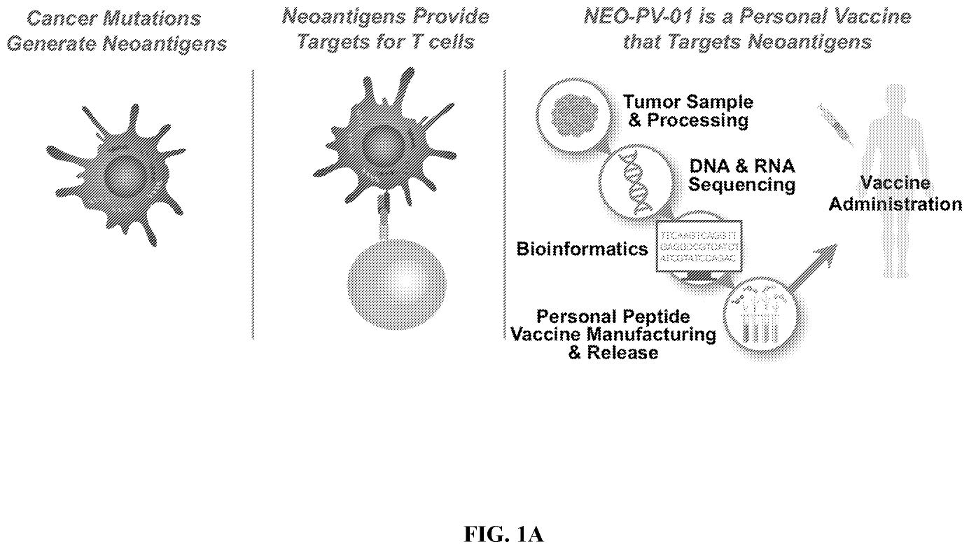

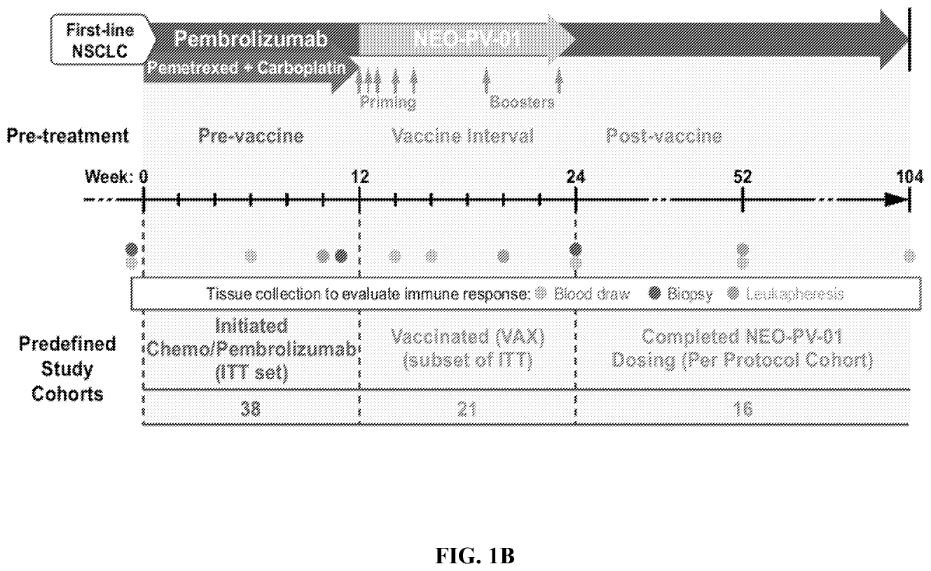

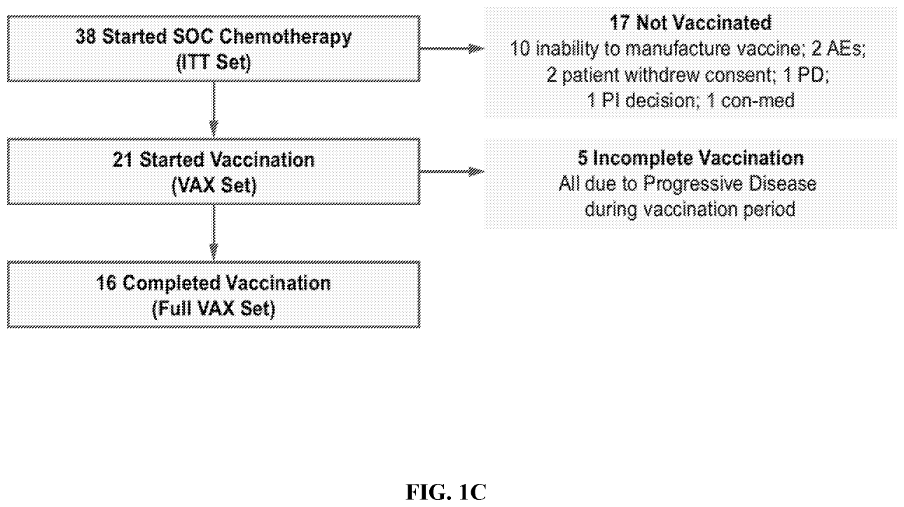

In addition to CTLA-4 and PD-1/PD-L1, numerous other immunomodulatory targets have been identified preliminarily, many with corresponding therapeutic antibodies that are being investigated in clinical trials. Page et al. (Annu Rev. Med. 2014.65) details targets of antibody immune modulators in FIG. 1, incorporated by reference herein.

The present disclosure features in exemplary aspects, novel combinations of a neoplasia vaccine or immunogenic composition and one or more inhibitors of the PD-L1 pathway. In some embodiments, the inhibitor of the PD-1 pathway is an anti-PD-L1 antibody, for example Pembrolizumab.

The present disclosure also features in other exemplary aspects, novel combinations of a neoplasia vaccine or immunogenic composition in combination with anti-PD-L1 antibody Pembrolizumab and one or more chemotherapeutic agents. In one embodiment, the one or more chemotherapeutic agents comprises a platinum-based anticancer therapeutic agent. A platinum-based anticancer agent can be selected from carboplatin, dicycloplatin, oxaliplatin, satraplatin and nedaplatin for use in treating cancer in combination with a neoplasia vaccine or immunogenic composition and Pembrolizumab. or a pharmaceutically acceptable salt or solvate thereof. In certain preferred embodiments, the platinum-based anticancer agent is Carboplatin. In some embodiments, the one or more chemotherapeutic agents comprises a first chemotherapeutic agent Carboplatin, and a second chemotherapeutic agent. In some embodiments, the second chemotherapeutic agent is an antimetabolite. In some embodiments, antimetabolite is Pemetrexed. In some embodiments, the novel combinations further comprise an adjuvant.

In some embodiments, a PD-L1 inhibitor such as pembrolizumab can be administered once or more than once along with neoantigen administration. In some dosage regimens, a PD-L1 inhibitor such as can be administered once as a priming dose at the beginning of the vaccination period, followed by one, two, three, four, five or more boost doses during and/or after the neoantigen vaccine doses. In some embodiments, a pembrolizumab dose is administered once before administering the neoantigen vaccine. In some embodiments, a pembrolizumab dose is administered more than once before administering the neoantigen vaccine. In some embodiments, a pembrolizumab dose is administered once after administering the neoantigen vaccine. In some embodiments, a pembrolizumab dose is administered more than once after administering the neoantigen vaccine. In some embodiments, a pembrolizumab dose is administered twice, three times, four times, five times or more after administering the neoantigen vaccine. In some embodiments, a nivolumab dose is administered both before and after administering the neoantigen vaccine.

In some embodiments the adjuvant is Hiltolol. In some embodiments the adjuvant is Poly-ICLC. In some embodiments, Hiltolol and Poly-ICLC are used.

It has been increasingly recognized that somatic mutations in tumor cells can lead to presentation of neoantigens, which can be recognized by the host immune system and directly lead to tumor cell killing (Ott and Wu 2019; Ott et al. 2017; Keskin et al. 2019; Hu et al. 2021). Numerous clinical trials have attempted to harness this mechanism of inducing tumor control by utilizing personalized neoantigen-based vaccination, particularly in the setting of solid tumors with a high neoantigen load, with some promising success in the adjuvant setting (Ott et al. 2017; Keskin et al. 2019; Sahin et al. 2017; Hilf et al. 2019). Inventors previously reported on a similar personalized neoantigen-based vaccine trial in combination with anti-PD1 in patients with melanoma, non-small cell lung cancer (NSCLC) and urothelial carcinoma (TCC) of the bladder, in which safety and feasibility was demonstrated, as well as the ability to deeply characterize immune responses generated against the vaccine peptides (Ott et al. 2020).

Treatment of patients with NSCLC by utilizing immunotherapeutic treatment modalities has been a relatively recent development, with an encouraging effect on prognosis for patients diagnosed with this disease. Several critical studies have led to the approval of immune checkpoint inhibitors (ICIs), including the PD-1 inhibitor Pembrolizumab, either as monotherapies or in combination with standard-of-care (SOC) chemotherapeutic regimens (Gettinger et al. 2015; Gandhi et al. 2018). Studies assessing the combination of Pembrolizumab plus chemotherapy resulted in increased progression free survival (PFS) from 4.9 months to 9.0 months in non-squamous NSCLC and from 4.8 months to 6.4 months in squamous NSCLC. (Gandhi et al. 2018; Gadgeel et al. 2020) (Borghaei et al. 2020). However, despite these successes, there is still clearly an urgent need for additional therapeutic options to further extend PFS for this patient population. Given the incomplete successes observed in the setting of ICIs alone or in combination with chemotherapy, there is a strong rationale to combine with neoantigen-based vaccination strategies in order to further boost the anti-tumor immune response in a targeted fashion.

The addition of neoantigen vaccine to a regimen of chemotherapy and pembrolizumab presents an opportunity to induce and expand tumor specific CD4⁺ and CD8⁺ T cell responses. The use of neoantigen vaccine as therapy in late-stage NSCLC in combination with anti PD-1 has been reported by us (Ott et al. 2020) with robust generation of both CD4⁺ and CD8⁺ responses in all patients. In addition, neoantigen vaccine has been studied in combination with tyrosine kinase inhibitor in a recent small study of 16 patients in late-stage NSCLC harboring EGFR mutations (Li et al. 2021). Both studies have shown such an approach of adding neoantigen vaccine is feasible, safe and leads to additional tumor specific immunity.

In some embodiments, the subject is suffering from a neoplasia selected from the group consisting of Non-Hodgkin's Lymphoma (NHL), clear cell Renal Cell Carcinoma (ccRCC), melanoma, sarcoma, leukemia or a cancer of the bladder, colon, brain, breast, head and neck, endometrium, lung, ovary, pancreas or prostate. In some embodiments, the neoplasia is metastatic melanoma. In some embodiments, the subject has no detectable neoplasia but is at high risk for disease recurrence. In embodiments, the cancer is selected from the group consisting of: adrenal, bladder, breast, cervical, colorectal, glioblastoma, head and neck, kidney chromophobe, kidney clear cell, kidney papillary, liver, lung adenocarcinoma, lung squamous, ovarian, pancreatic, melanoma, stomach, uterine corpus endometrial, and uterine carcinosarcoma. In embodiments, the cancer is selected from the group consisting of: prostate cancer, bladder, lung squamous, NSCLC, breast, head and neck, lung adenocarcinoma, GBM, Glioma, CML, AML, supretentorial ependyomas, acute promyelocytic leukemia, solitary fibrous tumors, and crizotinib resistant cancer. In embodiments, the cancer is selected from the group consisting of: CRC, head and neck, stomach, lung squamous, lung adenocarcinoma, prostate, bladder, stomach, renal cell carcinoma, and uterine. In embodiments, the cancer is selected from the group consisting of: melanoma, lung squamous, DLBCL, uterine, head and neck, uterine, liver, and CRC. In embodiments, the cancer is selected from the group consisting of: lymphoid cancer; Burkitt lymphoma, neuroblastoma, prostate adenocarcinoma, colorectal adenocarcinoma; Uterine/Endometrium Adenocarcinoma; MSI+; endometrium serous carcinoma; endometrium carcinosarcoma-malignant mesodermal mixed tumour; glioma; astrocytoma; GBM, acute myeloid leukemia associated with MDS; chronic lymphocytic leukemia-small lymphocytic lymphoma; myelodysplastic syndrome; acute myeloid leukemia; luminal NS carcinoma of breast; chronic myeloid leukemia; ductal carcinoma of pancreas; chronic myelomonocytic leukemia; myelofibrosis; myelodysplastic syndrome; prostate adenocarcinoma; essential thrombocythaemia; and medullomyoblastoma. In embodiments, the cancer is selected from the group consisting of: colorectal, uterine, endometrial, and stomach. In embodiments, the cancer is selected from the group consisting of: cervical, head and neck, anal, stomach, Burkitt's lymphoma, and nasopharyngeal carcinoma. In embodiments, the cancer is selected from the group consisting of: bladder, colorectal, and stomach. In embodiments, the cancer is selected from the group consisting of: lung, CRC, melanoma, breast, NSCLC, and CLL. In embodiments, the subject is a partial or non-responder to checkpoint inhibitor therapy. In embodiments, the subject is a partial or non-responder to CD40 agonist therapy, In embodiments, the cancer is selected from the group consisting of: bladder urothelial carcinoma (BLCA), breast invasive carcinoma (BRCA), breast cancer, cervical squamous cell carcinoma and endocervical adenocarcinoma (CESC), chronic lymphocytic leukemia (CLL), colorectal cancer (CRC), glioblastoma multiforme (GBM), head and neck squamous cell carcinoma (HNSC), kidney renal papillary cell carcinoma (KIRP), liver hepatocellular carcinoma (LIHC), lung adenocarcinoma (LUAD), lung squamous cell carcinoma (LUSC), pancreatic adenocarcinoma (PAAD), Prostate Cancer, skin cutaneous melanoma (SKCM), stomach adenocarcinoma (STAD), thyroid adenocarcinoma (THCA), and uterine corpus endometrioid carcinoma (UCEC). In embodiments, the cancer is selected from the group consisting of: colorectal cancer, uterine cancer, endometrium cancer, stomach cancer, and Lynch syndrome. In embodiments, the cancer is an MSI+ cancer.

### V. Pharmaceutical Compositions/Methods of Delivery

The present disclosure is also directed to pharmaceutical compositions comprising an effective amount of one or more compounds according to the present disclosure (including a pharmaceutically acceptable salt, thereof), optionally in combination with a pharmaceutically acceptable carrier, excipient or additive.

When administered as a combination, the therapeutic agents (i.e. the neoplasia vaccine or immunogenic composition and one or more inhibitors, such as one or more checkpoint inhibitors or the chemotherapeutic drug) can be formulated as separate compositions that are given at the same time or different times, or the therapeutic agents can be given as a single composition.

The compositions can be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compositions of the disclosure can be used to treat diseases and disease conditions that are acute, and can also be used for treatment of chronic conditions. In particular, the compositions of the disclosure are used in methods to treat or prevent a neoplasia.

In certain embodiments, the compounds of the disclosure are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it can be advantageous for the compounds of the disclosure to be administered for the remainder of the patient's life. In some embodiments, the patient is monitored to check the progression of the disease or disorder, and the dose is adjusted accordingly. In some embodiments, treatment according to the disclosure is effective for at least two weeks, three weeks, one month, two months, three months, four months, five months, six months, one year, two years, three years, four years, or five years, ten years, fifteen years, twenty years, or for the remainder of the subject's life.

As described herein, in certain embodiments, administration of the inhibitor is initiated before initiation of administration of the neoplasia vaccine or immunogenic composition. In other embodiments, administration of the inhibitor is initiated after initiation of administration of the neoplasia vaccine or immunogenic composition. In still other embodiments, administration of the inhibitor is initiated simultaneously with the initiation of administration of the neoplasia vaccine or immunogenic composition.

Administration of the inhibitor, such as a checkpoint inhibitor or a chemotherapeutic drug, can continue every 2, 3, 4, 5, 6, 7, 8 or more weeks after the first administration of the inhibitor, such as a checkpoint inhibitor or the chemotherapeutic drug. It is understood that week 1 is meant to include days 1-7, week 2 is meant to include days 8-14, week 3 is meant to include days 15-21 and week 4 is meant to include days 22-28. When dosing is described as being on weekly intervals it means approximately 7 days apart although in any given week the day can be one or more days before or after the scheduled day.

In certain embodiments, administration of the inhibitor, such as a checkpoint inhibitor or a chemotherapeutic drug, is withheld during the week prior to administration of the neoplasia vaccine or immunogenic composition. In other embodiments, administration of the inhibitor, such as a checkpoint inhibitor is withheld during administration of the neoplasia vaccine or immunogenic composition.

Surgical resection uses surgery to remove abnormal tissue in cancer, such as mediastinal, neurogenic, or germ cell tumors, or thymoma. In other embodiments, administration of the neoplasia vaccine or immunogenic composition is initiated 1, 2, 3, 4, 5, 6, 7, 8, 9, 10, 11, 12, 13, 14, 15 or more weeks after tumor resection. In some embodiments, administration of the neoplasia vaccine or immunogenic composition is initiated 4, 5, 6, 7, 8, 9, 10, 11 or 12 weeks after tumor resection.

Prime/boost regimens refer to the successive administrations of a vaccine or immunogenic or immunological compositions. In certain embodiments, administration of the neoplasia vaccine or immunogenic composition is in a prime/boost dosing regimen, for example administration of the neoplasia vaccine or immunogenic composition at weeks 1, 2, 3 or 4 as a prime and administration of the neoplasia vaccine or immunogenic composition is at months 2, 3 or 4 as a boost. In another embodiment heterologous prime-boost strategies are used to elicit a greater cytotoxic T-cell response (see Schneider et al., Induction of CD8+ T cells using heterologous prime-boost immunization strategies, Immunological Reviews Volume 170, Issue 1, pages 29-38, August 1999). In another embodiment DNA encoding neoantigens is used to prime followed by a protein boost. In another embodiment protein is used to prime followed by boosting with a virus encoding the neoantigen. In another embodiment a virus encoding the neoantigen is used to prime and another virus is used to boost. In another embodiment protein is used to prime and DNA is used to boost. In some embodiments, a DNA vaccine or immunogenic composition is used to prime a T-cell response and a recombinant viral vaccine or immunogenic composition is used to boost the response. In some embodiments, a viral vaccine or immunogenic composition is co-administered with a protein or DNA vaccine or immunogenic composition to act as an adjuvant for the protein or DNA vaccine or immunogenic composition. The patient can then be boosted with either the viral vaccine or immunogenic composition, protein, or DNA vaccine or immunogenic composition (see Hutchings et al., Combination of protein and viral vaccines induces potent cellular and humoral immune responses and enhanced protection from murine malaria challenge. Infect Immun, 2007 December; 75(12):5519-26. Epub 2007 Oct. 1).

As used herein, the term “fixed intermittent dosing regimen” refers to repeating cycles of preplanned drug administration in which the drug is administered on one or more consecutive days (“days on”) followed by one or more consecutive days of rest on which the drug is not administered (“days off”).

In some embodiments, the cycles are regular, in that the pattern of days on and days off is the same in each cycle. In some embodiments, the cycles are irregular, in that the pattern of days on and days off differs from one cycle to the next cycle. In some embodiments, each of the repeating cycles, however, is preplanned in that it is not determined solely in response to the appearance of one or more adverse events. In some embodiments, administration of the composition comprising the first component and/or the second component is repeated for one to ten cycles, such as for example one cycle, two cycles, three cycles, four cycles, five cycles, six cycles, seven cycles, eight cycles, nine cycles or ten cycles.

In some embodiments, a cycle comprises 3 days to 60 days. In some embodiments, a cycle comprises 7 to 50 days, such as 7 to 30 days, 7 to 21 days, or 7 to 14 days. In some embodiments, a cycle consists of 7 days.

In some embodiments, the fixed intermittent dosing regimen comprises a repeating cycle of administration of an effective amount of said composition comprising the first component and/or the second component on 1 to 5 consecutive days, such as 2 to 5 consecutive days, followed by 6 to 2 days of rest, such as 5 to 2 days of rest. In some embodiments, the fixed intermittent dosing regimen comprises a repeating cycle of administration of an effective amount of said composition comprising the first component and/or the second component on 5 consecutive days followed by 2 days of rest. In some embodiments, the fixed intermittent dosing regimen comprises a repeating cycle of administration of an effective amount of said composition comprising the first component and/or the second component on 4 consecutive days followed by 3 days of rest. In some embodiments, the fixed intermittent dosing regimen comprises a repeating cycle of administration of an effective amount of said composition comprising the first component and/or the second component on 3 consecutive days followed by 4 days of rest.

In some embodiments, the fixed intermittent dosing regimen comprises a repeating cycle of administration of an effective amount of said composition comprising the first component and/or the second component on 1 to 5 consecutive days, such as 2 to 5 consecutive days, followed by 6 to 2 days of rest, such as 5 to 2 days of rest. In some embodiments, placebo is administered on said days of rest.

The pharmaceutical compositions can be processed in accordance with conventional methods of pharmacy to produce medicinal agents for administration to patients in need thereof, including humans and other mammals.

Modifications of the neoantigenic peptides can affect the solubility, bioavailability and rate of metabolism of the peptides, thus providing control over the delivery of the active species. Solubility can be assessed by preparing the neoantigenic peptide and testing according to known methods well within the routine practitioner's skill in the art.

It has been found that a pharmaceutical composition comprising succinic acid or a pharmaceutically acceptable salt thereof (succinate) can provide improved solubility for the neoantigenic peptides. Thus, in one aspect, the disclosure provides a pharmaceutical composition comprising: at least one neoantigenic peptide or a pharmaceutically acceptable salt thereof; a pH modifier (such as a base, such as a dicarboxylate or tricarboxylate salt, for example, a pharmaceutically acceptable salt of succinic acid or citric acid); and a pharmaceutically acceptable carrier. Such pharmaceutical compositions can be prepared by combining a solution comprising at least one neoantigenic peptide with a base, such as a dicarboxylate or tricarboxylate salt, such as a pharmaceutically acceptable salt of succinic acid or citric acid (such as sodium succinate), or by combining a solution comprising at least one neoantigenic peptide with a solution comprising a base, such as a dicarboxylate or tricarboxylate salt, such as a pharmaceutically acceptable salt of succinic acid or citric acid (including, e.g., a succinate buffer solution). In certain embodiments, the pharmaceutical composition comprises sodium succinate. In certain embodiments, the pH modifier (such as citrate or succinate) is present in the composition at a concentration from about 1 mM to about 10 mM, and, in certain embodiments, at a concentration from about 1.5 mM to about 7.5 mM, or about 2.0 to about 6.0 mM, or about 3.75 to about 5.0 mM.

In certain embodiments of the pharmaceutical composition the pharmaceutically acceptable carrier comprises water. In certain embodiments, the pharmaceutically acceptable carrier further comprises dextrose. In certain embodiments, the pharmaceutically acceptable earner further comprises dimethylsulfoxide. In certain embodiments, the pharmaceutical composition further comprises an immunomodulator or adjuvant. In certain embodiments, the immunomodulator or adjuvant is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In certain embodiments, the immunomodulator or adjuvant comprises poly-ICLC.

Xanthenone derivatives such as, for example, Vadimezan or AsA404 (also known as 5,6-dimethylaxanthenone-4-acetic acid (DMXAA)), can also be used as adjuvants according to embodiments of the disclosure. Alternatively, such derivatives can also be administered in parallel to the vaccine or immunogenic composition of the disclosure, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5,6-Di-methylxanthenone-4-Acetic Acid, journal of Immunology, 190:5216-25 and Kim et al. (2013) Anticancer Flavonoids are Mouse-Selective STING Agonists, 8: 1396-1401).

The vaccine or immunological composition can also include an adjuvant compound chosen from the acrylic or methacrylic polymers and the copolymers of maleic anhydride and an alkenyl derivative. It is in particular a polymer of acrylic or methacrylic acid cross-linked with a polyalkenyl ether of a sugar or polyalcohol (carbomer), in particular cross-linked with an allyl sucrose or with allylpentaerythritol. It can also be a copolymer of maleic anhydride and ethylene cross-linked, for example, with divinyl ether (see U.S. Pat. No. 6,713,068 hereby incorporated by reference in its entirety).

In certain embodiments, the pH modifier can stabilize the adjuvant or immunomodulator as described herein.

In certain embodiments, a pharmaceutical composition comprises: one to five peptides, dimethyl sulfoxide (DMSO), dextrose, water, succinate, poly I: poly C, poly-L-lysine, carboxymethylcellulose, and chloride. In certain embodiments, each of the one to five peptides is present at a concentration between 200 μg/ml and 500 μg/ml, 300-400 μg/ml. In certain embodiments, the pharmaceutical composition comprises S 3% DMSO by volume, about 4-5% DMSO. In certain embodiments, the pharmaceutical composition comprises 3.5-5.5% dextrose, 4.9-5.0% dextrose in water. In certain embodiments, the pharmaceutical composition comprises δ5.0 mM succinate, 3.6-3.7 mM succinate (e.g., as sodium succinate). In certain embodiments, the pharmaceutical composition comprises ≥0.4 mg/ml poly I: poly C, for example, 1.0-2.2 mg/ml, for example, 1.7-1.9 mg/ml. In certain embodiments, the pharmaceutical composition comprises ≥0.375 mg/ml poly-L-Lysine, 0.5-2.0 mg/ml, or 1.5 mg/ml. In certain embodiments, the pharmaceutical composition comprises ≥1.25 mg/ml sodium carboxymethylcellulose, 2-7 mg/ml, for example, 4-5 mg/ml. In certain embodiments, the pharmaceutical composition comprises ≥0.225% sodium chloride, 0.5-1.0% sodium chloride, or 0.8-2.0% sodium chloride.

Pharmaceutical compositions comprise the herein-described tumor specific neoantigenic peptides in a therapeutically effective amount for treating diseases and conditions (e.g., a neoplasia/tumor), which have been described herein, optionally in combination with a pharmaceutically acceptable additive, carrier and/or excipient. One of ordinary skill in the art from this disclosure and the knowledge in the art will recognize that a therapeutically effective amount of one of more compounds according to the present disclosure can vary with the condition to be treated, its severity, the treatment regimen to be employed, the pharmacokinetics of the agent used, as well as the patient (animal or human) treated.

To prepare the pharmaceutical compositions according to the present disclosure, a therapeutically effective amount of one or more of the compounds according to the present disclosure can be intimately admixed with a pharmaceutically acceptable carrier according to conventional pharmaceutical compounding techniques to produce a dose. A carrier can take a wide variety of forms depending on the form of preparation desired for administration, e.g., ocular, oral, topical or parenteral, including gels, creams ointments, lotions and time released implantable preparations, among numerous others. In preparing pharmaceutical compositions in oral dosage form, any of the usual pharmaceutical media can be used. Thus, for liquid oral preparations such as suspensions, elixirs and solutions, suitable carriers and additives including water, glycols, oils, alcohols, flavoring agents, preservatives, coloring agents and the like can be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like can be used. If desired, the tablets or capsules can be enteric-coated or sustained release by standard techniques.

The active compound is included in the pharmaceutically acceptable earner or diluent in an amount sufficient to deliver to a patient a therapeutically effective amount for the desired indication, without causing serious toxic effects in the patient treated.

Oral compositions generally include an inert diluent or an edible carrier. They can be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material herein discussed, a liquid carrier such as fatty oil. In addition, dosage unit forms can contain various other materials which modify the physical form of the dosage unit, for example, coatings of sugar, shellac, or enteric agents.

Formulations of the present disclosure suitable for oral administration can be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, surface-active or dispersing agent. Molded tablets can be made by molding in a suitable machine a mixture of the powdered compound moistened with an inert liquid diluent. The tablets optionally can be coated or scored and can be formulated so as to provide slow or controlled release of the active ingredient therein.

Methods of formulating such slow or controlled release compositions of pharmaceutically active ingredients, are known in the art and described in several issued US Patents, some of which include, but are not limited to, U.S. Pat. Nos. 3,870,790; 4,226,859; 4,369,172: 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof can also be administered as a component of an elixir, suspension, syrup, wafer, chewing gum or the like. A syrup can contain, in addition to the active compounds, sucrose or fructose as a sweetening agent and certain preservatives, dyes and colorings and flavors.

Solutions or suspensions used for ocular, parenteral, intradermal, subcutaneous, or topical application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose.

In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS), and 5% dextrose in water (D5W). In certain embodiments, the aqueous solvent further comprises dimethyl sulfoxide (DMSO), e.g., in an amount of about 1-4%, or 2-3%. In certain embodiments, the pharmaceutically acceptable carrier is isotonic (i.e., has substantially the same osmotic pressure as a body fluid such as plasma).

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations are within the ambit of the skilled artisan in view of this disclosure and the knowledge in the art.

A skilled artisan from this disclosure and the knowledge in the art recognizes that in addition to tablets, other dosage forms can be formulated to provide slow or controlled release of the active ingredient. Such dosage forms include, but are not limited to, capsules, granulations and gel-caps.

Liposomal suspensions can also be pharmaceutically acceptable carriers. These can be prepared according to methods known to those skilled in the art. For example, liposomal formulations can be prepared by dissolving appropriate lipid(s) in an inorganic solvent that is then evaporated, leaving behind a thin film of dried lipid on the surface of the container. An aqueous solution of the active compound can then be introduced into the container. The container is then swirled by hand to free lipid material from the sides of the container and to disperse lipid aggregates, thereby forming the liposomal suspension. Other methods of preparation well known by those of ordinary skill can also be used in this aspect of the present disclosure.

The formulations can conveniently be presented in unit dosage form and can be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid earners or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin can be presented as ointments, creams, gels and pastes comprising the ingredient to be administered in a pharmaceutical acceptable carrier. A topical delivery system that can be used includes is a transdermal patch containing the ingredient to be administered.

Formulations for rectal administration can be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which stuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration can be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, carriers can include, for example, physiological saline or phosphate buffered saline (PBS).

For parenteral formulations, the carrier usually comprises sterile water or aqueous sodium chloride solution, though other ingredients including those which aid dispersion can be included. Of course, where sterile water is to be used and maintained as sterile, the compositions and carriers are also sterilized. Injectable suspensions can also be prepared, in which case appropriate liquid carriers, suspending agents and the like can be employed.

Formulations suitable for parenteral administration include aqueous and non-aqueous sterile injection solutions which can contain antioxidants, buffers, bacteriostats and solutes which render the formulation isotonic with the blood of the intended recipient; and aqueous and non-aqueous sterile suspensions which can include suspending agents and thickening agents. The formulations can be presented in unit-dose or multi-dose containers, for example, sealed ampules and vials, and can be stored in a freeze-dried (lyophilized) condition requiring only the addition of the sterile liquid carrier, for example, water for injections, immediately prior to use. Extemporaneous injection solutions and suspensions can be prepared from sterile powders, granules and tablets of the kind previously described.

Administration of the active compound can range from continuous (intravenous drip) to several oral administrations per day (for example, Q.I.D.) and can include oral, topical, eye or ocular, parenteral, intramuscular, intravenous, sub-cutaneous, transdermal (which can include a penetration enhancement agent), buccal and suppository administration, among other routes of administration, including through an eye or ocular route.

The neoplasia vaccine or immunogenic composition and the at least one inhibitor, such as a checkpoint inhibitor or chemotherapeutic agent, and any additional agents, can be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

Various techniques can be used for providing the subject compositions at the site of interest, such as injection, use of catheters, trocars, projectiles, pluronic gel, stents, sustained drag release polymers or other device which provides for internal access. Where an organ or tissue is accessible because of removal from the patient, such organ or tissue can be bathed in a medium containing the subject compositions, the subject compositions can be painted onto the organ, or can be applied in any convenient way.

The tumor specific neoantigenic peptides can be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

The tumor specific neoantigenic peptides can be utilized in combination with at least one known other therapeutic agent, or a pharmaceutically acceptable salt of said agent. Examples of known therapeutic agents which can be used for combination therapy include, but are not limited to, corticosteroids (e.g., cortisone, prednisone, dexamethasone), non-steroidal anti-inflammatory drugs (NSAIDS) (e.g., ibuprofen, celecoxib, aspirin, indomethicin, naproxen), alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; and/or RNA/DNA antimetabolites such as 5-azacytidine, 5-fiuorouracii and methotrexate; DNA antimetabolites such as 5-fluoro-2′-deoxy-uridine, ara-C, hydroxyurea and thioguanine; antibodies such as BERCEPT1N and RITUXAN.

In certain embodiments, administration of the compositions described herein can be combined with the administration of antagonists that block the release of histamine and anti-inflammatory drugs to prevent adverse allergic reactions. H1 and H2 antagonists can be administered to the patient before the administration of the compositions described herein.

It should be understood that in addition to the ingredients particularly mentioned herein, the formulations of the present disclosure can include other agents conventional in the art having regard to the type of formulation in question, for example, those suitable for oral administration can include flavoring agents.

Pharmaceutically acceptable salt forms can be the chemical form of compounds according to the present disclosure for inclusion in pharmaceutical compositions according to the present disclosure.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present disclosure which retain the desired biological activity of the parent compound and exhibit limited toxicological effects to normal cells. Non-limiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

The compounds herein are commercially available or can be synthesized. As can be appreciated by the skilled artisan, further methods of synthesizing the compounds of the formulae herein is evident to those of ordinary skill in the art. Additionally, the various synthetic steps can be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

### VI. Dosage

When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the disclosure can be varied so as to obtain an amount of the active ingredient which is effective to achieve the desired therapeutic response for a particular patient, composition, and mode of administration, without being toxic to the patient. Generally, agents or pharmaceutical compositions of the disclosure are administered in an amount sufficient to reduce or eliminate symptoms associated with viral infection and/or autoimmune disease.

A dose of an agent can be the maximum that a patient can tolerate and not develop serious or unacceptable side effects.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with viral infection or autoimmune disease) is observed in the treated subject, with minimal or acceptable toxic side effects. Applicable methods for determining an appropriate dose and dosing schedule for administration of a pharmaceutical composition of the present disclosure are described, for example, in Goodman and Oilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

Unit dosage formulations can be those containing a daily dose or unit, daily sub-dose, as herein discussed, or an appropriate fraction thereof, of the administered ingredient.

The dosage regimen for treating a disorder or a disease with the tumor specific neoantigenic peptides of this disclosure and/or compositions of this disclosure is based on a variety of factors, including the type of disease, the age, weight, sex, medical condition of the patient, the severity of the condition, the route of administration, and the particular compound employed. Thus, the dosage regimen can vary widely, but can be determined routinely using standard methods.

The amounts and dosage regimens administered to a subject can depend on a number of factors, such as the mode of administration, the nature of the condition being treated, the body weight of the subject being treated and the judgment of the prescribing physician; all such factors being within the ambit of the skilled artisan from this disclosure and the knowledge in the art. In some embodiments, an initial series of closely spaced immunizations can be administered to induce an immune response followed by a period of rest to allow memory T cells to be established and booster immunizations to expand the response. Alternatively, the priming doses can be administered over a longer period of time, and boosts can be administered more frequently for a longer period. For instance, a long priming period can be followed by boosts administered every 2 months for 1 year.

The amount of compound included within therapeutically active formulations according to the present disclosure is an effective amount for treating the disease or condition.

In general, a therapeutically effective amount of a compound in dosage form can range from slightly less than about 0.025 mg/kg/day to about 2.5 g/kg/day, about 0.1 mg/kg/day to about 100 mg/kg/day of the patient or considerably more, depending upon the compound used, the condition or infection treated and the route of administration, although exceptions to this dosage range can be contemplated by the present disclosure. In some embodiments, compounds according to the present disclosure are administered in amounts ranging from about 1 mg/kg/day to about 100 mg/kg/day. The dosage of the compound can depend on the condition being treated, the particular compound, and other clinical factors such as weight and condition of the patient and the route of administration of the compound. It is to be understood that the present disclosure has application for both human and veterinary use.

According to certain exemplary embodiments, the vaccine or immunogenic composition is administered at a dose of about 10 μg-1 mg per neoantigenic peptide. According to certain exemplary embodiments, the NEO-PV-01 vaccine/Adjuvant+pembrolizumab+chemotherapy regime comprises administering the vaccine or immunogenic composition at an average weekly dose level of about 10 μg-2000 μg per neoantigenic peptide. In some cases, a single dose of one or more neoantigenic peptides has a concentration between 100 μg/ml to 1000 μg/ml, 300-600 μg/ml, or 400-500 μg/ml. According to some embodiments, the NEO-PV-01 vaccine/Adjuvant+pembrolizumab+chemotherapy regime comprises administering Pembrolizumab at a dose of 200 mg by intravenous infusion (IV) plus chemotherapy with carboplatin (AUC 5)+pemetrexed (500 mg/m\hat{ }2) every 3 weeks for 4 cycles. At Week 12, all patients, regardless of their disease status, will receive NEO-PV-01+adjuvant administered subcutaneously (one vial of pooled peptides per injection site) in up to four distinct sites (each extremity or flanks) while continuing therapy with Pembrolizumab.

In some embodiments, a subject is administered neoantigenic peptides at a dosage of 10 μg to 2,000 μg per peptide. In some embodiments, a subject is administered neoantigenic peptides at a dosage of at least 10 μg, 50 μg, 100 μg, 150 μg, 200 μg, 250 μg, 300 μg, 400 μg, 500 μg, 600 μg, 800 μg, 1000 μg or 1500 μg per peptide. In some embodiments, a subject is administered neoantigenic peptides at a dosage of at most 2,000 μg, 1500 μg, 1000 μg, 800 μg, 700 μg, 600 μg, 500 μg, 400 μg, 300 μg, 250 μg, 200 μg, 100 μg, 75 μg per peptide. In some embodiments, a subject is administered neoantigenic peptides at a dosage of 10 μg to 50 μg, 10 μg to 100 μg, 10 μg to 200 μg, 10 μg to 300 μg, 10 μg to 400 μg, 10 μg to 500 μg, 10 μg to 600 μg, 10 μg to 800 μg, 10 μg to 1,000 μg, 10 μg to 1,500 μg, 10 μg to 2,000 μg, 50 μg to 100 μg, 50 μg to 200 μg, 50 μg to 300 μg, 50 μg to 400 μg, 50 μg to 500 μg, 50 μg to 600 μg, 50 μg to 800 μg, 50 μg to 1,000 μg, 50 μg to 1,500 μg, 50 μg to 2,000 μg, 100 μg to 200 μg, 100 μg to 300 μg, 100 μg to 400 μg, 100 μg to 500 μg, 100 μg to 600 μg, 100 μg to 800 μg, 100 μg to 1,000 μg, 100 μg to 1,500 μg, 100 μg to 2,000 μg, 200 μg to 300 μg, 200 μg to 400 μg, 200 μg to 500 μg, 200 μg to 600 μg, 200 μg to 800 μg, 200 μg to 1,000 μg, 200 μg to 1,500 μg, 200 μg to 2,000 μg, 300 μg to 400 μg, 300 μg to 500 μg, 300 μg to 600 μg, 300 μg to 800 μg, 300 μg to 1,000 μg, 300 μg to 1,500 μg, 300 μg to 2,000 μg, 400 μg to 500 μg, 400 μg to 600 μg, 400 μg to 800 μg, 400 μg to 1,000 μg, 400 μg to 1,500 μg, 400 μg to 2,000 μg, 500 μg to 600 μg, 500 μg to 800 μg, 500 μg to 1,000 μg, 500 μg to 1,500 μg, 500 μg to 2,000 μg, 600 μg to 800 μg, 600 μg to 1,000 μg, 600 μg to 1,500 μg, 600 μg to 2,000 μg, 800 μg to 1,000 μg, 800 μg to 1,500 μg, 800 μg to 2,000 μg, 1,000 μg to 1,500 μg, 1,000 μg to 2,000 μg, or 1,500 μg to 2,000 μg per peptide. In some embodiments, a subject is administered neoantigenic peptides at a dosage of 10 μg, 50 μg, 100 μg, 200 μg, 300 μg, 400 μg, 500 μg, 600 μg, 800 μg, 1,000 μg, 1,500 μg, or 2,000 μg per peptide.

In some embodiments, a subject is administered Pembrolizumab at a dosage of 50 mg to 400 mg. In some embodiments, a subject is administered Pembrolizumab at a dosage of at least 50 mg, 75 mg, 100 mg, 150 mg, 200 mg, 220 mg, 240 mg, 250 mg, 260 mg, 270 mg, 280 mg, 300 mg, 320 mg or 350 mg. In some embodiments, a subject is administered Pembrolizumab at a dosage of at most 400 mg, 350 mg, 300 mg, 260 mg, 240 mg, 200 mg, 150 mg, 100 mg or 75 mg. In some embodiments, a subject is administered Pembrolizumab at a dosage of 50 mg to 75 mg, 50 mg to 100 mg, 50 mg to 150 mg, 50 mg to 200 mg, 50 mg to 220 mg, 50 mg to 240 mg, 50 mg to 260 mg, 50 mg to 280 mg, 50 mg to 300 mg, 50 mg to 350 mg, 50 mg to 400 mg, 75 mg to 100 mg, 75 mg to 150 mg, 75 mg to 200 mg, 75 mg to 220 mg, 75 mg to 240 mg, 75 mg to 260 mg, 75 mg to 280 mg, 75 mg to 300 mg, 75 mg to 350 mg, 75 mg to 400 mg, 100 mg to 150 mg, 100 mg to 200 mg, 100 mg to 220 mg, 100 mg to 240 mg, 100 mg to 260 mg, 100 mg to 280 mg, 100 mg to 300 mg, 100 mg to 350 mg, 100 mg to 400 mg, 150 mg to 200 mg, 150 mg to 220 mg, 150 mg to 240 mg, 150 mg to 260 mg, 150 mg to 280 mg, 150 mg to 300 mg, 150 mg to 350 mg, 150 mg to 400 mg, 200 mg to 220 mg, 200 mg to 240 mg, 200 mg to 260 mg, 200 mg to 280 mg, 200 mg to 300 mg, 200 mg to 350 mg, 200 mg to 400 mg, 220 mg to 240 mg, 220 mg to 260 mg, 220 mg to 280 mg, 220 mg to 300 mg, 220 mg to 350 mg, 220 mg to 400 mg, 240 mg to 260 mg, 240 mg to 280 mg, 240 mg to 300 mg, 240 mg to 350 mg, 240 mg to 400 mg, 260 mg to 280 mg, 260 mg to 300 mg, 260 mg to 350 mg, 260 mg to 400 mg, 280 mg to 300 mg, 280 mg to 350 mg, 280 mg to 400 mg, 300 mg to 350 mg, 300 mg to 400 mg, or 350 mg to 400 mg. In some embodiments, a subject is administered Pembrolizumab at a dosage of 50 mg, 75 mg, 100 mg, 150 mg, 200 mg, 220 mg, 240 mg, 260 mg, 280 mg, 300 mg, 350 mg, or 400 mg.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient can be administered at once, or can be divided into a number of smaller doses to be administered at varying intervals of time.

The disclosure provides for pharmaceutical compositions containing at least one tumor specific neoantigen described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which can be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing viral infection and/or autoimmune disease.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container.

Methods for formulating the pharmaceutical compositions of the present disclosure are conventional and well known in the art (see Remington and Remington's). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th ed., Lippincott Williams & Wilkins), which is hereby incorporated by reference.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents, in the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet, can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well known in the art.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(orthoesters).

In embodiments, the active ingredients) are administered by aerosol. This is accomplished by preparing an aqueous aerosol, liposomal preparation, or solid particles containing the compound. A non-aqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present disclosure are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422, 119, 5,023,084, which are hereby incorporated by reference. The transdermal patch can also be any transdermal patch well known in the art, including transscrotal patches. Pharmaceutical compositions in such transdermal patches can contain one or more absorption enhancers or skin permeation enhancers well known in the art (see, e.g., U.S. Pat. Nos. 4,379,454 and 4,973,468, which are hereby incorporated by reference). Transdermal therapeutic systems for use in the present disclosure can be based on iontophoresis, diffusion, or a combination of these two effects.

Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flu can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g., gum acacia or gum tragacanth, naturally occurring phosphatides, e.g., soybean lecithin and sorbitan monooleate derivatives.

Examples of antioxidants include, but are not limited to, butylated hydroxy anisole (BHA), ascorbic acid and derivatives thereof tocopherol and derivatives thereof, and cysteine.

Examples of preservatives include, but are not limited to, trehalose, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylforamamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, TRANSCUTOL, and AZONE.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone.

In addition to the active ingredient(s), the ointments, pastes, creams, and gels of the present disclosure can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the disclosure in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well known in the art and are suitable for use in the present disclosure. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or can slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as SILASTIC, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilizing agents.

Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer can be a polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drag-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the herein-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

### VII. Vaccine or Immunogenic Compositions

The present disclosure is directed to methods of combination treatment. The combination treatment comprises at least an immunogenic composition, e.g., a neoplasia vaccine or immunogenic composition capable of raising a specific T-cell response. The neoplasia vaccine or immunogenic composition comprises neoantigenic peptides and/or neoantigenic polypeptides corresponding to tumor specific neoantigens identified by the methods described herein.

A suitable neoplasia vaccine or immunogenic composition can contain a plurality of tumor specific neoantigenic peptides. In an embodiment, the vaccine or immunogenic composition can include between 1 and 100 sets of peptides, between 1 and 50 such peptides, between 10 and 30 sets peptides, or between 15 and 25 peptides. According to another embodiment, the vaccine or immunogenic composition can include at least one peptides, such as 2, 3, 4, or 5 peptides, In certain embodiments, the vaccine or immunogenic composition can comprise 5, 6, 7, 8, 9, 10, 11, 12, 13, 14, 15, 16, 17, 18, 19, 20, 21, 22, 23, 24, 25, 26, 27, 28, 29, or 30 different peptides. Multiple doses of the vaccine or immunogenic composition can be administered to a subject. Each dose of the vaccine composition can comprise different sets of peptides. For instance, one dose or part of one dose of the composition can comprise 5 peptides. Another part of the dose can comprise a different set of 5 peptides.

The optimum amount of each peptide to be included in the vaccine or immunogenic composition and the optimum dosing regimen can be determined by one skilled in the art without undue experimentation. For example, the peptide or its variant can be prepared for intravenous (i.v.) injection, sub-cutaneous (s.c.) injection, intradermal (i.d.) injection, intraperitoneal (i.p.) injection, intramuscular (i.m.) injection. Methods of peptide injection include s.c, i.d., i.p., i.m., and i.v. Methods of DNA injection include i.d., i.m., s.c, i.p. and i.v. For example, doses of between 1 and 500 mg, 50 μg and 1.5 mg, or 10 μg to 500 μg, of peptide or DNA can be given and can depend from the respective peptide or DNA. Doses of this range were successfully used in previous trials (Brunsvig P F, et al., Cancer Immunol Immunother. 2006; 55(12): 1553-1564; M. Staehler, et al, ASCO meeting 2007; Abstract No 3017). Other methods of administration of the vaccine or immunogenic composition are known to those skilled in the art.

The vaccine or immunogenic composition can be administered to a subject in the form of one or more subcutaneous injections. In one embodiment, a single dose of the composition can be divided into one or more subcutaneous injections for administration. For instance, a single dose of the composition can be divided in to 1, 2, 3, 4, 5, 6, 7 or 8 different subcutaneous injections. Each injection of the composition can comprise one or more peptides. The peptides in each injection of a single dose of the composition can comprise different sets of peptides. In some examples, each injection of a single dose of the composition comprises 1, 2, 3, 4, 5 or 6 different peptides. Alternatively, each subcutaneous injection of a single dose of the composition can comprise the same set of peptides. In some cases, multiple injections, as part of a single dose of the vaccine or the immunogenic composition can comprise different sets of peptides. For instance, a single dose of the composition can be divided in to 4 injections with each injection comprising 5 different sets of peptides.

The vaccine or immunogenic composition can be administered to the patient as a subcutaneous injection in a single location. For example, a single dose of the vaccine or immunogenic composition can be administered to the patient as one injection in an extremity. In cases where a single dose of the composition is divided over one or more injections, the dose can be administered into a subject in multiple locations. For instance, a case where a dose is divided in to 4 different injections, the 4 different injections can be administered into different extremities. In some cases, multiple injections as part of a single dose of the composition can be administered at the same location at different time periods. For instance, a 5, 10, 15, 20, 30, 50 or 60 minute time period can be provided between different injections of one single dose of the vaccine or immunogenic composition.

In one embodiment of the present disclosure the different tumor specific neoantigenic peptides and/or polypeptides are selected for use in the neoplasia vaccine or immunogenic composition so as to maximize the likelihood of generating an immune attack against the neoplasia/tumor of the patient. Without wishing to be bound by theory, it is believed that the inclusion of a diversity of tumor specific neoantigenic peptides can generate a broad scale immune attack against a neoplasia/tumor. In one embodiment, the selected tumor specific neoantigenic peptides/polypeptides are encoded by missense mutations. In a second embodiment, the selected tumor specific neoantigenic peptides polypeptides are encoded by a combination of missense mutations and neoORF mutations. In a third embodiment, the selected tumor specific neoantigenic peptides/polypeptides are encoded by neoORF mutations.

In one embodiment in which the selected tumor specific neoantigenic peptides/polypeptides are encoded by missense mutations, the peptides and/or polypeptides are chosen based on their capability to associate with the particular MHC molecules of the patient. Peptides/polypeptides derived from neoORF mutations can also be selected on the basis of their capability to associate with the particular MHC molecules of the patient, but can also be selected even if not predicted to associate with the particular MHC molecules of the patient.

The vaccine or immunogenic composition is capable of raising a specific cytotoxic T cells response and/or a specific helper T-cell response.

The vaccine or immunogenic composition can further comprise an adjuvant and/or a carrier. Examples of useful adjuvants and carriers are given herein. The peptides and/or polypeptides in the composition can be associated with a carrier such as, e.g., a protein or an antigen-presenting cell such as e.g., a dendritic cell (DC) capable of presenting the peptide to a T-cell.

Adjuvants are any substance whose admixture into the vaccine or immunogenic composition increases or otherwise modifies the immune response to the mutant peptide. Carriers are scaffold structures, for example a polypeptide or a polysaccharide, to which the neoantigenic peptides, is capable of being associated. Optionally, adjuvants are conjugated covalently or non-covalently to the peptides or polypeptides of the disclosure.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant can also alter an immune response, for example, by changing a primarily humoral or Th2 response into a primarily cellular, or Th1 response.

Suitable adjuvants include, but are not limited to 1018 ISS, aluminum salts, Amplivax, AS 15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox, Quil or Superfos. Several immunological adjuvants (e.g., F59) specific for dendritic cells and their preparation have been described previously (Dupuis M, et al, Cell Immunol. 1998; 186(1): 18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al, J Immunother Emphasis Tumor Immunol. 1996 (6):414-418).

Toll like receptors (TLRs) can also be used as adjuvants, and are important members of the family of pattern recognition receptors (PRRs) which recognize conserved motifs shared by many micro-organisms, termed “pathogen-associated molecular patterns” (PAMPS). Recognition of these “danger signals” activates multiple elements of the innate and adaptive immune system. ‘TLRs are expressed by cells of the innate and adaptive immune systems such as dendritic cells (DCs), macrophages, T and B cells, mast cells, and granulocytes and are localized in different cellular compartments, such as the plasma membrane, lysosomes, endosomes, and endo lysosomes. Different TLRs recognize distinct PAMPS. For example, TLR4 is activated by LPS contained in bacterial cell walls, TLR 9 is activated by unmethylated bacterial or viral CpG DNA, and TLRS is activated by double stranded RNA. TLR ligand binding leads to the activation of one or more intracellular signaling pathways, ultimately resulting in the production of many key molecules associated with inflammation and immunity (particularly the transcription factor NF-κB and the Type-I interferons). TLR mediated DC activation leads to enhanced DC activation, phagocytosis, upregulation of activation and co-stimulation markers such as CD80, CD83, and CD86, expression of CCR7 allowing migration of DC to draining lymph nodes and facilitating antigen presentation to T cells, as well as increased secretion of cytokines such as type I interferons, IL-12, and IL-6. All of these downstream events are critical for the induction of an adaptive immune response.

Among the most promising cancer vaccine or immunogenic composition adjuvants currently in clinical development are the TLR9 agonist CpG and the synthetic double-stranded RNA (dsRNA) TLR3 ligand poly-ICLC. In preclinical studies poly-ICLC appears to be the most potent TLR adjuvant when compared to LPS and CpG due to its induction of pro-inflammatory cytokines and lack of stimulation of IL-IO, as well as maintenance of high levels of co-stimulatory molecules in IX s i. Furthermore, poly-ICLC was recently directly compared to CpG in non-human primates (rhesus macaques) as adjuvant for a protein vaccine or immunogenic composition consisting of human papillomavirus (HPV)16 capsomers (Stahl-Hennig C, Eisenblatter M, Jasny E, et al. Synthetic double-stranded R As are adjuvants for the induction of T helper I and humoral immune responses to human papillomavirus in rhesus macaques. PLoS pathogens. April 2009; 5(4)).

CpG immunostimulatory oligonucleotides have also been reported to enhance the effects of adjuvants in a vaccine or immunogenic composition setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Th1 cells and strong cytotoxic T-lymphocyte (CTL) generation, even in the absence of CD4 T-cell help. The Th1 bias induced by TLR9 stimulation is maintained even in the presence of vaccine adjuvants such as alum or incomplete Freund's adjuvant (IFA) that normally promote a Th2 bias. CpG oligonucleotides show even greater adjuvant activity when formulated or co-administered with other adjuvants or in formulations such as microparticles, nanoparticles, lipid, emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, June 2006, 471-484). U.S. Pat. No. 6,406,705 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which can be a component of the pharmaceutical composition of the present disclosure. Other TLR binding molecules such as RNA binding TLR 7, TLR 8 and/or TLR 9 can also be used.

Other examples of useful adjuvants include, but. are not limited to, chemically modified CpGs (e.g., CpR, Idera), Poly(I:C) (e.g., polyI:CI2U), non-CpG bacterial DNA or RNA as well as immunoactive small molecules and antibodies such as cyclophosphamide, sunitinib, bevacizumab, Celebrex, NCX-4016, sildenafil, tadalafil, vardenafil, sorafinib, XL-999, CP-547632, pazopanib, ZD2171, AZD2171, ipilimumab, tremelimumab, and SC58175, which can act therapeutically and/or as an adjuvant. The amounts and concentrations of adjuvants and additives useful in the context of the present disclosure can readily be determined by the skilled artisan without undue experimentation. Additional adjuvants include colony-stimulating factors, such as Granulocyte Macrophage Colony Stimulating Factor (GM-CSF, sargramostim).

Poly-ICLC is a synthetically prepared double-stranded RNA consisting of polyI and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′5′-OAS and the Pl/eIF2a kinase, also known as the PKR (4-6), as well as RIG-I helicase and MDA5.

In rodents and non-human primates, poly-ICLC was shown to enhance T cell responses to viral antigens, cross-priming, and the induction of tumor-, virus-, and autoantigen-specific CD8+ T-cells. In a recent study in non-human primates, poly-ICLC was found to be essential for the generation of antibody responses and T-cell immunity to DC targeted or non-targeted HIV Gag p24 protein, emphasizing its effectiveness as a vaccine adjuvant.

In human subjects, transcriptional analysis of serial whole blood samples revealed similar gene expression profiles among the 8 healthy human volunteers receiving one single subcutaneous (s.c.) administration of poly-ICLC and differential expression of up to 212 genes between these 8 subjects versus 4 subjects receiving placebo. Remarkably, comparison of the poly-ICLC gene expression data to previous data from volunteers immunized with the highly effective yellow fever vaccine YF17D showed that a large number of transcriptional and signal transduction canonical pathways, including those of the innate immune system, were similarly upregulated at peak time points.

More recently, an immunologic analysis was reported on patients with ovarian, fallopian tube, and primary peritoneal cancer in second or third complete clinical remission who were treated on a phase 1 study of subcutaneous vaccination with synthetic overlapping long peptides (OLP) from the cancer testis antigen NY-ESO-1 alone or with Montanide-ISA-51, or with 1.4 mg poly-ICLC and Montanide. The generation of NY-ESO-1-specific CD4+ and CD 8+ T-cell and antibody responses were markedly enhanced with the addition of poly-ICLC and Montanide compared to OLP alone or OLP and Montanide.

A vaccine or immunogenic composition according to the present disclosure can comprise more than one different adjuvant. Furthermore, the disclosure encompasses a therapeutic composition comprising any adjuvant substance including any of those herein discussed. It is also contemplated that the peptide or polypeptide, and the adjuvant can be administered separately in any appropriate sequence.

A carrier can be present independently of an adjuvant. The carrier can be covalently linked to the antigen. A carrier can also be added to the antigen by inserting DNA encoding the carrier in frame with DNA encoding the antigen. The function of a carrier can for example be to confer stability, to increase the biological activity, or to increase serum half-life. Extension of the half-life can help to reduce the number of applications and to lower doses, thus are beneficial for therapeutic but also economic reasons. Furthermore, a carrier can aid presenting peptides to T-cells. The carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier can be a physiologically acceptable carrier acceptable to humans and safe. However, tetanus toxoid and/or diphtheria toxoid are suitable carriers in one embodiment of the disclosure. Alternatively, the carrier can be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and APC is present. Correspondingly, it can enhance the immune response if not only the peptide is used for activation of CTLs, but if additionally APCs with the respective MHC molecule are added. Therefore, in some embodiments the vaccine or immunogenic composition according to the present disclosure additionally contains at least one antigen presenting cell.

The antigen-presenting cell (or stimulator cell) typically has an MHC class I or II molecule on its surface, and in one embodiment is substantially incapable of itself loading the MHC class I or II molecule with the selected antigen. As is described in more detail herein, the MHC class I or II molecule can readily be loaded with the selected antigen in vitro.

CD8+ cell activity can be augmented through the use of CD4+ cells. The identification of CD4 T+ cell epitopes for tumor antigens has attracted interest because many immune based therapies against cancer can be more effective if both CD8+ and CD4+ T lymphocytes are used to target a patient's tumor, CD4+ cells are capable of enhancing CD8 T cell responses. Many studies in animal models have clearly demonstrated better results when both CD4+ and CD8+ T cells participate in anti-tumor responses (see e.g., Nishimura et al. (1999) Distinct role of antigen-specific T helper type I (Th1) and Th2 cells in tumor eradication in vivo. J Ex Med 190:617-27). Universal CD4+ T cell epitopes have been identified that are applicable to developing therapies against different types of cancer (see e.g., Kobayashi et al. (2008) Current Opinion in Immunology 20:221-27). For example, an HLA-DR restricted helper peptide from tetanus toxoid was used in melanoma vaccines to activate CD4+ T cells nonspecifically (see e.g., Slingluff et al. (2007) Immunologic and Clinical Outcomes of a Randomized Phase II Trial of Two Multipeptide Vaccines for Melanoma in the Adjuvant Setting, Clinical Cancer Research 13(21):6386-95). It is contemplated within the scope of the disclosure that such CD4+ cells can be applicable at three levels that vary in their tumor specificity: 1) a broad level in which universal CD4+ epitopes (e.g., tetanus toxoid) can be used to augment CD 8+ cells; 2) an intermediate level in which native, tumor-associated CD4+ epitopes can be used to augment CD8+ cells; and 3) a patient specific level in which neoantigen CD4+ epitopes can be used to augment CD8+ cells in a patient specific manner.

CD8+ cell immunity can also be generated with neoantigen loaded dendritic cell (DC) vaccine. DCs are potent antigen-presenting cells that initiate T cell immunity and can be used as cancer vaccines when loaded with one or more peptides of interest, for example, by direct peptide injection. For example, patients that were newly diagnosed with metastatic melanoma were shown to be immunized against 3 HLA-A*0201-restricted gp100 melanoma antigen-derived peptides with autologous peptide pulsed CD40 L/IFN-g-activated mature DCs via an IL-12p70-producing patient DC vaccine (see e.g., Carreno et al (2013) L-12p70-producing patient DC vaccine elicits Tel-polarized immunity, Journal of Clinical Investigation, 123(8):3383-94 and Ali et al. (2009) In situ regulation of DC subsets and T cells mediates tumor regression in mice, Cancer Immunotherapy, 1(8): 1-10). It is contemplated within the scope of the disclosure that neoantigen loaded DCs can be prepared using the synthetic TLR 3 agonist Polyinosinic-Polycytidylic Acid-poly-L-lysine Carboxymethylcellulose (Poly-ICLC) to stimulate the DCs. Poly-ICLC is a potent individual maturation stimulus for human DCs as assessed by an upregulation of CD83 and CD86, induction of interleukin-12 (IL-I2), tumor necrosis factor (TNF), interferon gamma-induced protein 10 (IP-10), interleukin 1 (IL-1), and type I interferons (IFN), and minimal interleukin 10 (IL-10) production. DCs can be differentiated from frozen peripheral blood mononuclear cells (PBMCs) obtained by leukapheresis, while PBMCs can be isolated by Ficoll gradient centrifugation and frozen in aliquots.

Illustratively, the following 7 day activation protocol can be used. Day 1—PBMCs are thawed and plated onto tissue culture flasks to select for monocytes which adhere to the plastic surface after 1-2 hr incubation at 37° C. in the tissue culture incubator. After incubation, the lymphocytes are washed off and the adherent monocytes are cultured for 5 days in the presence of interleukin-4 (IL-4) and granulocyte macrophage-colony stimulating factor (GM-CSF) to differentiate to immature DCs. On Day 6, immature DCs are pulsed with the keyhole limpet hemocyanin (KLH) protein which serves as a control for the quality of the vaccine and can boost the immunogenicity of the vaccine. The DCs are stimulated to mature, loaded with peptide antigens, and incubated overnight. On Day 7, the cells are washed, and frozen in 1 ml aliquots containing 4-20×10(6) cells using a controlled-rate freezer. Lot release testing for the batches of DCs can be performed to meet minimum specifications before the DCs are injected into patients (see e.g., Sabado et al. (2013) Preparation of tumor antigen-loaded mature dendritic cells for immunotherapy, J. Vis Exp. August 1; (78). doi: 10.3791/50085).

A DC vaccine can be incorporated into a scaffold system to facilitate delivery to a patient. Therapeutic treatment of a patients neoplasia with a DC vaccine can utilize a biomaterial system that releases factors that recruit host dendritic cells into the device, differentiates the resident, immature DCs by locally presenting adjuvants (e.g., danger signals) while releasing antigen, and promotes the release of activated, antigen loaded DCs to the lymph nodes (or desired site of action) where the DCs can interact, with T cells to generate a potent cytotoxic T lymphocyte response to the cancer neoantigens. Implantable biomaterials can be used to generate a potent cytotoxic T lymphocyte response against a neoplasia in a patient specific manner. The biomaterial-resident dendritic cells can then be activated by exposing them to danger signals mimicking infection, in concert with release of antigen from the biomaterial. The activated dendritic cells then migrate from the biomaterials to lymph nodes to induce a cytotoxic T effector response. This approach has previously been demonstrated to lead to regression of established melanoma in preclinical studies using a lysate prepared from tumor biopsies (see e.g., Ali et al. (2209) In situ regulation of DC subsets and T cells mediates tumor regression in mice, Cancer Immunotherapy 1 (8): 1-10; Ali et al. (2009).

In some embodiments, the antigen presenting cells are dendritic cells. Suitably, the dendritic cells are autologous dendritic cells that are pulsed with the neoantigenic peptide. The peptide can be any suitable peptide that gives rise to an appropriate T-cell response. T-cell therapy using autologous dendritic cells pulsed with peptides from a tumor associated antigen is disclosed in Murphy et al. (1996) The Prostate 29, 371-380 and Tjua et al. (1997) The Prostate 32, 272-278.

Thus, in one embodiment of the present disclosure the vaccine or immunogenic composition containing at least one antigen presenting cell is pulsed or loaded with one or more peptides of the present disclosure. Alternatively, peripheral blood mononuclear cells (PBMCs) isolated from a patient can be loaded with peptides in vivo and injected back into the patient. As an alternative the antigen presenting cell comprises an expression construct encoding a peptide of the present disclosure. The polynucleotide can be any suitable polynucleotide and is capable of transducing the dendritic cell, thus resulting in the presentation of a peptide and induction of immunity.

The inventive pharmaceutical composition can be compiled so that the selection, number and/or amount of peptides present in the composition is/are tissue, cancer, and/or patient-specific. For instance, the exact selection of peptides can he guided by expression patterns of the parent proteins in a given tissue to avoid side effects. The selection can be dependent on the specific type of cancer, the status of the disease, earlier treatment regimens, the immune status of the patient, and, of course, the HLA-haplotype of the patient. Furthermore, the vaccine or immunogenic composition according to the disclosure can contain individualized components, according to personal needs of the particular patient. Examples include varying the amounts of peptides according to the expression of the related neoantigen in the particular patient, unwanted side-effects due to personal allergies or other treatments, and adjustments for secondary treatments following a first round or scheme of treatment.

Pharmaceutical compositions comprising the peptide of the disclosure can be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use can depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition and possibly by measuring specific CTL activity in the patient's blood. It should be kept in mind that the peptide and compositions of the present disclosure can generally be employed in serious disease states, that is, life-threatening or potentially life threatening situations, especially when the cancer has metastasized. For therapeutic use, administration should begin as soon as possible after the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. In some embodiments, the pharmaceutical compositions are administered parenterally, e.g., intravenously, subcutaneously, intradermally, or intramuscularly. The compositions can be administered at the site of surgical excision to induce a local immune response to the tumor. The disclosure provides compositions for parenteral administration which comprise a solution of the peptides and vaccine or immunogenic compositions are dissolved or suspended in an acceptable carrier, for example, an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

A liposome suspension containing a peptide can be administered intravenously, locally, topically, etc. in a dose which varies according to, inter alia, the manner of administration, the peptide being delivered, and the stage of the disease being treated. For targeting to the immune cells, a ligand, such as, e.g., antibodies or fragments thereof specific for cell surface determinants of the desired immune system cells, can be incorporated into the liposome.

For solid compositions, conventional or nanoparticle nontoxic solid carriers can be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the disclosure, and for example, at a concentration of 25%-75%.

For aerosol administration, the immunogenic peptides can be supplied in finely divided form along with a surfactant and propellant. Typical percentages of peptides are 0.01%-20% by weight, for example, 1%-10%. The surfactant can, of course, be nontoxic, and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides can be employed. The surfactant can constitute 0.1%-20% by weight of the composition, for example, 0.25-5%. The balance of the composition is ordinarily propellant. A carrier can also be included as desired, as with, e.g., lecithin for intranasal delivery.

The peptides and polypeptides of the disclosure can be readily synthesized chemically utilizing reagents that are free of contaminating bacterial or animal substances (Merrifield R B: Solid phase peptide synthesis. I. The synthesis of a tetrapeptide. J. Am. Chem, Soc. 85:2149-54, 1963).

The peptides and polypeptides of the disclosure can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the disclosure. Upon introduction into an acutely or chronically infected host or into a non-infected host, the vector expresses the immunogenic peptide, and thereby elicits a host CTL response.

For therapeutic or immunization purposes, nucleic acids encoding the peptide of the disclosure and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Generally, a plasmid for a vaccine or immunological composition can comprise DNA encoding an antigen (e.g., one or more neoantigens) operatively linked to regulatory sequences which control expression or expression and secretion of the antigen from a host cell, e.g., a mammalian cell; for instance, from upstream to downstream, DNA for a promoter, such as a mammalian virus promoter (e.g., a CMV promoter such as an hCMV or mCMV promoter, e.g., an early-intermediate promoter, or an SV40 promoter—see documents cited or incorporated herein for useful promoters), DNA for a eukaryotic leader peptide for secretion (e.g., tissue plasminogen activator), DNA for the neoantigen(s), and DNA encoding a terminator (e.g., the 3′ UTR transcriptional terminator from the gene encoding Bovine Growth Hormone or bGH polyA). A composition can contain more than one plasmid or vector, whereby each vector contains and expresses a different neoantigen. Mention is also made of Wasmoen U.S. Pat. No. 5,849,303, and Dale U.S. Pat. No. 5,811,104, whose text can be useful DNA or DNA plasmid formulations can be formulated with or inside cationic lipids; and, as to cationic lipids, as well as adjuvants, mention is also made of Loosmore U.S. Patent Application 2003/0104008. Also, teachings in Audonnet U.S. Pat. Nos. 6,228,846 and 6,159,477 can be relied upon for DNA plasmid teachings that can be employed in constructing and using DNA plasmids that contain and express i vivo.

The nucleic acids can also be delivered complexed to cationic compounds, such as cationic lipids. Lipid-mediated gene delivery methods are described, for instance, in WO 1996/18372; WO 1993/24640; Mannino & Gould-Fogerite, BioTechniques 6(7): 682-691 (1988); U.S. Pat. No. 5,279,833; WO 1991/06309; and Feigner et al., Proc. Natl. Acad. Sci. USA 84: 7413-7414 (1987).

RNA encoding the peptide of interest (e.g., mRNA) can also be used for delivery (see, e.g., Kiken et al, 2011; Su et al, 2011; see also U.S. Pat. No. 8,278,036; Halabi et al. J Clin Oncol (2003) 21: 1232-1237; Petsch et al, Nature Biotechnology 2012 Dec. 7; 30(12): 1210-6).

Information concerning poxviruses that can be used in the practice of the disclosure, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC. 1, NYVAC.2, MVA, VA-BN), canarypox virus (e.g., Wheatley C93 Strain, ALVAC), fowlpox virus (e.g., FP9 Strain, Webster Strain, TROVAC), dovepox, pigeonpox, quail pox, and raccoon pox, inter alia, synthetic or non-naturally occurring recombinants thereof, uses thereof, and methods for making and using such recombinants can be found in scientific and patent literature, such as: U.S. Pat. Nos. 4,603,112, 4,769,330, 5,110,587, 5,174,993, 5,364,773, 5,762,938, 5,494,807, 5,766,597, 7,767,449, 6,780,407, 6,537,594, 6,265,189, 6,214,353, 6,130,066, 6,004,777, 5,990,091, 5,942,235, 5,833,975, 5,766,597, 5,756,101, 7,045,313, 6,780,417, 8,470,598, 8,372,622, 8,268,329, 8,268,325, 8,236,560, 8,163,293, 7,964,398, 7,964,396, 7,964,395, 7,939,086, 7,923,017, 7,897,156, 7,892,533, 7,628,980, 7,459,270, 7,445,924, 7,384,644, 7,335,364, 7,189,536, 7,097,842, 6,913,752, 6,761,893, 6,682,743, 5,770,212, 5,766,882, and 5,989,562, and Panicali, D. Proc. Natl. Acad. Sci. 1982; 79; 4927-493, Panicali D. Proc. Natl. Acad. Sci. 1983; 80(17): 5364-8, Mackett, M. Proc. Natl. Acad. Sci. 1982; 79: 7415-7419, Smith G L. Proc, Natl. Acad. Sci. 1983; 80(23): 7155-9, Smith G L. Nature 1983; 302: 490-5, Sullivan V J. Gen. Vir. 1987; 68: 2587-98, Perkus M Journal of Leukocyte Biology 1995; 58: 1-13, Yilma T D. Vaccine 1989; 7: 484-485, Brochier B. Nature 1991; 354: 520-22, Wiktor, TJ. Proc. Natl Acad, Sci. 1984; 81:7194-8, Rupprecht, CE. Proc, Natl Acd. Sci. 1986; 83: 7947-50, Poulet, H Vaccine 2007; 25 (Jul.): 5606-12, Weyer J. Vaccine 2009; 27 (Nov.): 7198-201, Buller, RM Nature 1985; 317(6040): 813-5, Boiler R M. J. Virol. 1988; 62(3):866-74, Flexner, C. Nature 1987; 330(6145): 259-62, Shida, H. J. Virol. 1988; 62(12): 4474-80, Kotwal G J. J. Virol. 1989; 63(2): 600-6, Child, S J. Virology 1990; 174(2): 625-9, Mayr A. Zentralbl Bakteriol 1978; 167(5,6): 375-9, Antoine G. Virology. 1998; 244(2): 365-96, Wyatt, L S. Virology 1998; 251(2): 334-42, Sancho, MC. J. Virol. 2002; 76(16); 8313-34, Gallego-Gomez, JC. J. Virol. 2003; 77(19); 10606-22), Goebel S J. Virology 1990; (a,b) 179: 247-66, Tartaglia, J. Virol. 1992; 188(1): 217-32, Najera J L. J. Virol. 2006; 80(12): 6033-47, Najera, J L. J. Virol. 2006; 80: 6033-6047, Gomez, CE. J. Gen. Virol. 2007; 88: 2473-78, Mooij, P. Jour. Of Virol. 2008; 82: 2975-2988, Gomez, CE. Curr. Gene Ther. 2011; 11: 189-217, Cox, W. Virology 1993; 195: 845-50, Perkus, M. Jour. Of Leukocyte Biology 1995; 58: 1-13, Blanchard T J. J Gen Virology 1998; 79(5): 1159-67, Amara R. Science 2001; 292: 69-74, Hel, Z., J. Immunol. 2001; 167: 7180-9, Gherardi M M. J. Virol. 2003; 77: 7048-57, Didierlaurent, A. Vaccine 2004; 22: 3395-3403, Bissht H. Proc. Nat. Aca. Sci. 2004; 101: 6641-46, McCurdy L H. Clin. Inf. Dis 2004; 38: 1749-53, Earl P L. Nature 2004; 428: 182-85, Chen Z. J. Virol. 2005; 79: 2678-2688, Najera J L. J. Virol. 2006; 80(12): 6033-47, Nam J H. Acta. Virol. 2007; 51:125-30, Antonis A F. Vaccine 2007; 25: 4818-4827, B Weyer J. Vaccine 2007; 25: 4213-22, Ferrier-Rembert A. Vaccine 2008; 26(14): 1794-804, Corbett M. Proc. Natl. Acad. Sci. 2008: 105(6): 2046-51, Kaufman H L., J. Clin. Oncol. 2004; 22: 2122-32, Amato, RJ. Clin. Cancer Res. 2008; 14(22): 7504-10, Dreicer R. Invest New Drugs 2009; 27(4): 379-86, Kantoff P W. J. Clin. Oncol. 2010, 28, 1099-1 105, Amato R J. J. Clin. Can. Res. 2010; 16(22): 5539-47, Kim, D W. Hum. Vaccine. 2010: 6: 784-791, Oudard, S. Cancer Immunol.]mm another. 2011; 60: 261-71, Wyatt, L S. Aids Res. Hum. Retroviruses. 2004; 20: 645-53, Gomez, CE. Virus Research 2004; 105: 11-22, Webster, DP. Proc. Natl Acad. Sci. 2005; 102; 4836-4, Huang, X. Vaccine 2007; 25: 8874-84, Gomez, CE. Vaccine 2007a; 25: 2863-85, Esteban M. Hum. Vaccine 2009; 5: 867-871, Gomez, CE. Curr. Gene therapy 2008; 8(2): 97-120, Whelan, K T. PLoS One 2009; 4(6): 5934, Scriba, T J. Eur, Jour. Immuno. 2010; 40(1): 279-90, Corbett, M. Proc. Natl, Acad. Sci. 2008; 105: 2046-2051, Midgley, CM. J. Gen. Virol. 2008; 89: 2992-97, Von Krempelhuber, A. Vaccine 2010; 28: 1209-16, Perreau, M. J. Of Virol. 2011; October: 9854-62, Pantaleo, G. Curr Opin HIV-AIDS. 2010; 5: 391-3%, each of which is incorporated herein by reference.

As to adenovirus vectors useful in the practice of the disclosure, mention is made of U.S. Pat. No. 6,955,808. The adenovirus vector used can be selected from the group consisting of the Ad5, Ad35, Ad 11, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Ad11 vectors are described in U.S. Pat. No. 6,913,922. C6 adenovirus vectors are described in U.S. Pat. Nos. 6,780,407; 6,537,594; 6,309,647; 6,265,189; 6,156,567; 6,090,393; 5,942,235 and 5,833,975. C7 vectors are described in U.S. Pat. No. 6,277,558. Adenovirus vectors that are El-defective or deleted, E3-d elective or deleted, and/or E4-defective or deleted can also be used. Certain adenoviruses having mutations in the El region have improved safety margin because El-defective adenovirus mutants are replication-defective in non-permissive cells, or, at the very least, are highly attenuated. Adenoviruses having mutations in the E3 region can have enhanced the immunogenicity by disrupting the mechanism whereby adenovirus down-regulates MHC class I molecules. Adenoviruses having E4 mutations can have reduced immunogenicity of the adenovirus vector because of suppression of late gene expression. Such vectors can be particularly useful when repeated re-vaccination utilizing the same vector is desired. Adenovirus vectors that are deleted or mutated in El, E3, E4, El and E3, and El and E4 can be used in accordance with the present disclosure. Furthermore, “gutless” adenovirus vectors, in which ail viral genes are deleted, can also be used in accordance with the present disclosure. Such vectors require a helper virus for their replication and require a special human 293 cell line expressing both Ela and Cre, a condition that does not exist in natural environment. Such “gutless” vectors are non-immunogenic and thus the vectors can be inoculated multiple times for re-vaccination. The “gutless” adenovirus vectors can be used for insertion of heterologous inserts/genes such as the transgenes of the present disclosure, and can even be used for co-delivery of a large number of heterologous inserts/genes.

As to lentivirus vector systems useful in the practice of the disclosure, mention is made of U.S. Pat. Nos. 6,428,953, 6,165,782, 6,013,516, 5,994,136, 6,312,682, and 7,198,784, and documents cited therein.

With regard to AAV vectors useful in the practice of the disclosure, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein.

Another vector is BCG (Bacille Calmette Guerin). BCG vectors are described in Stover et al. (Nature 351:456-460 (1991)). A wide variety of other vectors useful for therapeutic administration or immunization of the peptides of the disclosure, e.g., Salmonella typhi vectors and the like, is apparent to those skilled in the art from the description herein.

Vectors can be administered so as to have in vivo expression and response akin to doses and/or responses elicited by antigen administration.

In some embodiments, a means of administering nucleic acids encoding the peptide of the disclosure uses minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g., poly-alanine) or naturally-occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Several vector elements are required: a promoter with a down-stream cloning site for minigene insertion; a polyadenylation signal for efficient transcription termination; an E. coli origin of replication; and an E. coli selectable marker (e.g., ampicillin or kanamycin resistance). Numerous promoters can be used for this purpose, e.g., the human cytomegalovirus (hCMV) promoter. See, U.S. Pat. Nos. 5,580,859 and 5,589,466 for other suitable promoter sequences.

Additional vector modifications can be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immuno stimulatory sequences (ISSs or CpGs) play a role in the immunogenicity of DNA' vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used. Examples of proteins or polypeptides that could beneficially enhance the immune response if co-expressed include cytokines (e.g., IL2, 11,12, GM-CSF), cytokine-inducing molecules (e.g., LeIF) or costimulatory molecules. Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g., TGF-β) can be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted herein, nucleic acids are conveniently formulated with cationic lipids. In addition, glycolipids, fusogenic liposomes, peptides and compounds referred to collectively as protective, interactive, non-condensing (P1NC) could also be complexed to purified plasmid DNA to influence variables such as stability, intramuscular dispersion, or trafficking to specific organs or cell types.

Target cell sensitization can be used as a functional assay for expression and MHC class I presentation of minigene-encoded CTL epitopes. The plasmid DNA is introduced into a mammalian cell line that is suitable as a target for standard CTL chromium release assays. The transfection method used is dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 labeled and used as target cells for epitope-specific CTL lines. Cytolysis, detected by 51 Cr release, indicates production of MHC presentation of mini gene-encoded CTL epitopes.

In vivo immunogenicity is a second approach for functional testing of minigene DNA formulations. Transgenic mice expressing appropriate human MHC molecules are immunized with the DNA product. The dose and route of administration are formulation dependent (e.g., IM for DNA in PBS, IP for lipid-complexed DNA). Twenty-one days after immunization, splenocytes are harvested and restimulated for 1 week in the presence of peptides encoding each epitope being tested. These effector cells (CTLs) are assayed for cytolysis of peptide-loaded, chromium-51 labeled target cells using standard techniques. Lysis of target cells sensitized by MHC loading of peptides corresponding to minigene-encoded epitopes demonstrates DNA vaccine function for in vivo induction of CTLs.

Peptides can be used to elicit CTL ex vivo, as well. The resulting CTL, can be used to treat chronic tumors in patients in need thereof that do not respond to other conventional forms of therapy, or does not respond to a peptide vaccine approach of therapy. Ex vivo CTL responses to a particular tumor antigen are induced by incubating in tissue culture the patient's CTL precursor cells (CTLp) together with a source of antigen-presenting cells (APC) and the appropriate peptide. After an appropriate incubation time (typically 1-4 weeks), in which the CTLp are activated and mature and expand into effector CTL, the cells are infused back into the patient, where they destroy their specific target cell (i.e., a tumor cell). In order to optimize the in vitro conditions for the generation of specific cytotoxic T cells, the culture of stimulator cells are maintained in an appropriate serum-free medium.

Prior to incubation of the stimulator cells with the cells to be activated, e.g., precursor CD 8+ cells, an amount of antigenic peptide is added to the stimulator cell culture, of sufficient quantity to become loaded onto the human Class I molecules to be expressed on the surface of the stimulator cells. In the present disclosure, a sufficient amount of peptide is an amount that allows about 200, or 200 or more, human Class I MHC molecules loaded with peptide to be expressed on the surface of each stimulator cell. In some embodiments, the stimulator cells are incubated with >2 μg/ml peptide. For example, the stimulator cells are incubated with >3, 4, 5, 10, 15, or more μg/ml peptide.

Resting or precursor CD8+ cells are then incubated in culture with the appropriate stimulator cells for a time period sufficient to activate the CD8+ cells. In some embodiments, the CD8+ cells are activated in an antigen-specific manner. The ratio of resting or precursor CD8+(effector) cells to stimulator cells can vary from individual to individual and can further depend upon variables such as the amenability of an individual's lymphocytes to culturing conditions and the nature and severity of the disease condition or other condition for which the within-described treatment modality is used. The lymphocyte: stimulator cell ratio can be in the range of about 30:1 to 300:1, The effector/stimulator culture can be maintained for as long a time as is necessary to stimulate a therapeutically useable or effective number of CD8+ cells.

The induction of CTL in vitro requires the specific recognition of peptides that are bound to allele specific MHC class I molecules on APC. The number of specific MHC/peptide complexes per APC is crucial for the stimulation of CTL, particularly in primary immune responses. While small amounts of peptide/MHC complexes per cell are sufficient to render a cell susceptible to lysis by CTL, or to stimulate a secondary CTL response, the successful activation of a CTL precursor (pCTL) during primary response requires a significantly higher number of MHC/peptide complexes. Peptide loading of empty major histocompatibility complex molecules on cells allows the induction of primary cytotoxic T lymphocyte responses.

Since mutant cell lines do not exist for every human MHC allele, it is advantageous to use a technique to remove endogenous MHC-associated peptides from the surface of APC, followed by loading the resulting empty MHC molecules with the immunogenic peptides of interest. The use of non-transformed (non-tumorigenic), non-infected cells, and autologous cells of patients as APC is desirable for the design of CTL induction protocols directed towards development of ex vivo CTL therapies. This application discloses methods for stripping the endogenous MHC-associated peptides from the surface of APC followed by the loading of desired peptides.

A stable MHC class I molecule is a trimeric complex formed of the following elements: 1) a peptide usually of 8-10 residues, 2) a transmembrane heavy polymorphic protein chain which bears the peptide-binding site in its a1 and a2 domains, and 3) a non-covalently associated non-polymorphic light chain, p2microglobuiin. Removing the bound peptides and/or dissociating the p2microglobulin from the complex renders the MHC class I molecules nonfunctional and unstable, resulting in rapid degradation. All MHC class I molecules isolated from PBMCs have endogenous peptides bound to them. Therefore, the first step is to remove all endogenous peptides bound to MHC class I molecules on the APC without causing their degradation before exogenous peptides can be added to them.

Two possible ways to free up MHC class I molecules of bound peptides include lowering the culture temperature from 37° C. to 26° C. overnight to destabilize p2microgiobulin and stripping the endogenous peptides from the cell using a mild acid treatment. The methods release previously bound peptides into the extracellular environment allowing new exogenous peptides to bind to the empty class I molecules. The cold-temperature incubation method enables exogenous peptides to bind efficiently to the MHC complex, but requires an overnight incubation at 26° C. which can slow the cell's metabolic rate, it is also likely that cells not actively synthesizing MHC molecules (e.g., resting PBMC) would not produce high amounts of empty surface MHC molecules by the cold temperature procedure.

Harsh acid stripping involves extraction of the peptides with trifluoroacetic acid, pH 2, or acid denaturation of the immunoaffinity purified class I-peptide complexes. These methods are not feasible for CTL induction, since it is important to remove the endogenous peptides while preserving APC viability and an optimal metabolic slate which is critical for antigen presentation. Mild acid solutions of pH 3 such as glycine or citrate-phosphate buffers have been used to identify endogenous peptides and to identify tumor associated T cell epitopes. The treatment is especially effective, in that only the MHC class I molecules are destabilized (and associated peptides released), while other surface antigens remain intact, including MHC class II molecules. Most importantly, treatment of cells with the mild acid solutions does not affect the cell's viability or metabolic state. The mild acid treatment is rapid since the stripping of the endogenous peptides occurs in two minutes at 4° C. and the APC is ready to perform its function after the appropriate peptides are loaded. The technique is utilized herein to make peptide-specific APCs for the generation of primary antigen-specific CTL. The resulting APC are efficient in inducing peptide-specific CD8+ CTL.

Activated CD8+ cells can be effectively separated from the stimulator cells using one of a variety of known methods. For example, monoclonal antibodies specific for the stimulator cells, for the peptides loaded onto the stimulator cells, or for the CD8+ cells (or a segment thereof). can be utilized to bind their appropriate complementary ligand. Antibody-tagged molecules can then be extracted from the stimulator-effector cell admixture via appropriate means, e.g., via well-known immunoprecipitation or immunoassay methods.

Effective, cytotoxic amounts of the activated CD 8+ cells can vary between in vitro and in vivo uses, as well as with the amount and type of cells that are the ultimate target of these killer cells. The amount can also vary depending on the condition of the patient and should be determined via consideration of ail appropriate factors by the practitioner. About 1×10⁶ to about 1×10¹², about 1×10⁸ to about 1×10¹¹, or about 1×10⁹ to about 1×10¹⁰ activated CD8+ cells can be utilized for adult humans, compared to about 5×10⁶-5×10⁷ cells used in mice.

As discussed herein, the activated CD8+ cells are harvested from the cell culture prior to administration of the CD8+ cells to the individual being treated. It is important to note, however, that unlike other present and proposed treatment modalities, the present method uses a cell culture system that is not tumorigenic. Therefore, if complete separation of stimulator cells and activated CD8+ cells are not achieved, there is no inherent danger known to be associated with the administration of a small number of stimulator cells, whereas administration of mammalian tumor-promoting cells can be extremely hazardous.

Methods of re-introducing cellular components are known in the art and include procedures such as those exemplified in U.S. Pat. No. 4,844,893 to Honsik, et al. and U.S. Pat. No. 4,690,915 to Rosenberg. For example, administration of activated CD8+ cells via intravenous infusion is appropriate.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning; A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Wei, 1996); “Gene Transfer Vectors for Mammalian Cells” (Miller and Calos, 1987); “Current Protocols in Molecular Biology” (Ausubel, 1987); “PGR: The Polymerase Chain Reaction”, (Mullis, 1994); “Current Protocols in Immunology” (Coligan, 1991). These techniques are applicable to the production of the polynucleotides and polypeptides of the disclosure, and, as such, can be considered in making and practicing the disclosure. Particularly useful techniques for particular embodiments are discussed in the sections that follow.

### VIII. Therapeutic Methods

The present disclosure provides methods of inducing a neoplasia/tumor specific immune response in a subject, vaccinating against a neoplasia/tumor, treating and or alleviating a symptom of cancer in a subject by administering the subject a neoplasia vaccine or a neoantigenic peptide or composition of the disclosure and at least one inhibitor, such as a checkpoint inhibitor or chemotherapeutic agent.

In particular, the present disclosure is directed to methods of treating or preventing a neoplasia comprising the steps of administering to a subject (a) a neoplasia vaccine or immunogenic composition, and (b) at least one inhibitor, such as a checkpoint inhibitor or chemotherapeutic agent.

In one aspect, provided herein is a method of treating or preventing a cancer in a human subject in need thereof comprising administering to the human subject in need thereof: a first component comprising: (i) a polypeptide comprising a cancer-specific neoepitope of a protein expressed by cancer cells of the cancer, (ii) a polynucleotide encoding the polypeptide of (i), (iii) one or more APCs comprising the polypeptide of (i) or the polynucleotide of (ii), (iv) a T cell receptor (TCR) specific for a complex comprising an HLA protein expressed by the human subject and a cancer-specific neoepitope of a protein expressed by cancer cells of the cancer neoepitope, or (v) T cells comprising the TCR of (iv); and (a) a second component comprising an anti-cancer agent which is an antibody or an antigen-binding portion thereof that binds specifically to a Programmed Death-1 (PD-1) receptor and inhibits PD-1 activity; and (b) a third component comprising a platinum-based chemotherapy; wherein the human subject: (i) has not previously received a systemic treatment for metastatic disease, (ii) has not previously received an immunotherapy with an anti-PD-1 antibody, and (iii) has not previously received an immunotherapy with an anti-PD-L1 antibody.

According to the disclosure, the herein-described neoplasia vaccine or immunogenic composition can be used for a patient that has been diagnosed as having cancer, or at risk of developing cancer.

The described combination of the disclosure is administered in an amount sufficient to induce a CTL response.

The vaccine or immunogenic composition comprising neoantigenic peptides can be administered to a subject for the treatment of a condition. The subject can be administered one or more inhibitors, such as a checkpoint inhibitor or a chemotherapeutic drug, in addition to the neoantigenic peptides. Administration of the one or more inhibitors, such as a checkpoint inhibitor or chemotherapeutic drug, can be performed before the administration of the neoantigenic peptides. In some embodiments, more than one inhibitor, such as a checkpoint inhibitor or chemotherapeutic drug, is being administered to the patient. In such cases, one inhibitor, such as a checkpoint inhibitor or chemotherapeutic drug, can be administered before the administration of the neoantigen peptides. For instance, in a case where a combination of neoantigenic peptides.

Patients can be screened before administration and after the administration of the compositions described herein. Patients can undergo screening assessments that document historical health status as well as their current and future health status in general and as related to their underlying disease. The screening assessments can include tests like vital signs (including diastolic and systolic blood pressure, heart rate, temperature, weight, and height), electrocardiograms, symptom directed physical exam, hematology (including hematocrit, hemoglobin, RBC count, WBC count with differential, and platelet count), chemistry (including tests for glucose, urea nitrogen, creatinine, sodium, potassium, calcium, total and direct bilirubin, AST, ALT, alkaline phosphatase, lactate dehydrogenase (LDH), and adrenocorticotropic hormone), liver function tests (such as detecting levels of AST, ALT, total and direct bilirubin), pregnancy testing, CT or MRI, surgical or core needle biopsy of a primary or metastatic tumor site for DNA and RNA sequencing, immunological analysis. Biopsies can be used to evaluate the presence of T-cell infiltrates in the tumor and their localization with respect to tumor margins by tests like immunohistochemistry, western blot analysis, RNA and DNA analysis. The presence of tumor-associated macrophages and DCs within the tumor micro-environment can also be evaluated. The list of markers for analysis can include, but not be limited to, the following: CD3, CD4, CD8, CD45RO, PD-L1, PD-1, FoxP3, Granzyme B, Perforin, CD68, CD163, MHC Class I, MHC Class II, CD83 AND CD11b.

Immune response parameters can be analyzed for changes over time from baseline levels before the administration of the treatments and can include summaries of characterization of nucleic acids (e.g., DNA mutations, transcript abundance), histopathology, and immune cell analyses in tissues obtained at the Pretreatment, Pre-vaccination Treatment, and Vaccination Treatment phases as well as at the time of preliminary assessment. Reporting the test results can include tables depicting shifts from earlier time points in order to compare changes in immune parameters. Descriptive statistics and frequency distributions can be used as appropriate. Immunological analyses can include summaries for CD8+ and CD4+ T-cell response measured by ex vivo IFN-γ ELISpot and assessed through spot counts. Analyses can be used to assess changes from pretreatment to pre-vaccination treatment to vaccination treatment to preliminary assessment. Reporting can include medians and inter-quartile ranges, as well as tables depicting shifts from earlier time points for each patient. Additionally, nonparametric tests (e.g., Wilcoxon signed-rank test) can be used to determine differences in the ELISpot data between time points as appropriate. Fold changes in biomarkers measured on a continuous scale can be summarized and compared across response categories using the Wilcoxon rank-sum test between treatment arms for every cohort. For multi-gene assays, genes can be grouped into analysis sets to characterize biological functions of the cells as appropriate.

Primary objectives can include: The rate of adverse events and severe adverse events leading to treatment discontinuation [Time Frame: Baseline through 90 days after the last dose of pembrolizumab] Rate of adverse events and severe adverse events leading to treatment discontinuation and those adverse events and severe adverse events detected during symptom-directed physical examinations (changes in safety laboratory evaluations, physical examination findings. vital signs, and ECOG PS.

Secondary Outcome measures can include:
1. Date of the first documented PD based on Objective Response Rate (ORR), defined as the proportion of patients who achieve complete response (CR) or partial response (PR) based on Response Criteria in Solid Tumors (RECIST) v1.1.
2. Clinical Benefit Rate (CBR), defined as the proportion of patients who achieve a CR, PR, or stable disease (SD) based on RECIST v1.1.
3. Duration of Response, DOR, defined as the date of the first documentation of a confirmed response to the date of the first documented PD based on RECIST v1.1.
4. Response Conversion Rate (RCR), defined as the proportion of patients who improve in RECIST v1.1 category subsequent to vaccination (e.g., PD to SD/PR/CR, SD to PR/CR, PR/CR).
5. Progression Free Survival (PFS), defined as the time from the date of first dosing to the date of first documented PD or death.
6. Overall Survival (OS), defined from the date of enrollment and death from any cause.

Tests and results can include a detailed characterization of the phenotype and abundance of antigen-specific T cells, both in the periphery and in the tumor microenvironment. The abundance of regulatory cells such as regulatory T cells or myeloid-derived suppressor cells, and T-cell recognition, activation, and cytotoxicity can also be evaluated with PBMCs and tumor cells. Additionally, ex vivo induction of neoantigen T-cell responses can also be performed on peripheral blood and leukapheresis samples. Presence of circulating tumor DNA (ctDNA) and vaccine-specific antibody responses can be evaluated after treatment with the compositions described herein.

In one aspect, provided herein is a method of treating or preventing a cancer in a human subject in need thereof comprising administering to the human subject in need thereof: (a) a first component comprising: (i) a polypeptide comprising a cancer-specific neoepitope of a protein expressed by cancer cells of the cancer, (ii) a polynucleotide encoding the polypeptide of (i), (iii) one or more APCs comprising the polypeptide of (i) or the polynucleotide of (ii), (iv) a T cell receptor (TCR) specific for a complex comprising an HLA protein expressed by the human subject and a cancer-specific neoepitope of a protein expressed by cancer cells of the cancer neoepitope, or (v) T cells comprising the TCR of (iv); and (b) a second component comprising an anti-cancer agent which is an antibody or an antigen-binding portion thereof that binds specifically to a Programmed Death-1 (PD-1) receptor and inhibits PD-1 activity; and (c) a third component comprising a platinum-based chemotherapy; wherein: (i) the method promotes epitope spread of an epitope that is different than any of the cancer-specific neoepitopes; (ii) a median progression-free survival (PFS) of a first population of human subjects with the cancer treated with the first, second and third components is longer than a median PFS of a second population of subjects with the cancer treated with the second and/or third components but not the first component; (iii) an overall response rate (ORR) of a first population of human subjects with the cancer treated with the first, second and third components is higher than an ORR of a second population of subjects with the cancer treated with the second and/or third components but not the first component; (iv) a percentage of subjects with at least a 12-month progression-free survival (PFS) of a first population of human subjects with the cancer treated with the first, second and third components is higher than a percentage of subjects with at least a 12-month PFS of a second population of subjects with the cancer treated with the second and/or third components but not the first component; (v) a median overall survival (OS) of a first population of human subjects with the cancer treated with the first, second and third components is longer than a median OS of a second population of subjects with the cancer treated with the second and/or third components but not the first component; (vi) a percentage of subjects who achieve complete response, partial response, prolonged stable disease or stable disease for 6 months or more (CBR) of a first population of human subjects with the cancer treated with the first, second and third components is higher than an CBR of a second population of subjects with the cancer treated with the second and/or third components but not the first component; (vii) reduction of tumor size in a first population of human subjects with the cancer treated with the first, second and third components is greater than reduction of tumor size in a second population of subjects with the cancer treated with the second and/or third components but not the first component; (viii) a level of CD4+ T cells that infiltrate a tumor in a first population of human subjects treated with the first, second and third components is higher than a level of CD4+ T cells that infiltrate a tumor in a second population of subjects treated with the second and/or third components but not the first component; (ix) a level of effector and cytotoxic CD4+ T cells generated in a first population of human subjects treated with the first, second and third components is higher than a level of effector and cytotoxic CD4+ T cells generated in a second population of subjects treated with the second and/or third components but not the first component; (x) the method increases a level of CD4+ T cells specific to a cancer-specific neoepitope that upregulate expression of ZEB2, PDCD1, TOX, TIGT, CXCR3, ITGB1, GZMA and/or ICOS; (xi) the method increases a level of CD4+/CD62Lhi/CD69+/CD27+/CCR7+ T cells specific to a cancer-specific neoepitope; and/or (xii) the method increases a level of CD4+/NKG7+/CCL4+/CCL5+/GNLY+/LAG3+ T cells specific to a cancer-specific neoepitope.

For inclusion in a clinical trial the following criteria were followed: Ages Eligible for Study: 18 Years and older (Adult, Older Adult); Sexes Eligible for Study: All. Adverse Events (NCI CTCAE) version 4.03, Grade of 0 or 1, except for toxicities not considered a safety risk (eg, alopecia or vitiligo). Screening laboratory values must meet the following criteria and should be obtained within 30 days (or 45 days if a biopsy is repeated) prior to study treatment:
1. White blood cell (WBC) count ≥3×10e3/μL
2. Absolute neutrophil count (ANC) ≥1.5×10e3/μL
3. Platelet count ≥100×10e3/μL
4. Hemoglobin >9 g/dL
5. Serum creatinine ≤1.5× upper limit of normal (ULN) or creatinine clearance (CrCl) ≥40 mL/min/1.73 m\hat{ }2
6. Aspartate aminotransferase (AST) and alanine aminotransferase (ALT) ≤2.5×ULN or ≤5×ULN for patients with liver metastases
7. Total bilirubin ≤1.5×ULN (except in patients with Gilbert Syndrome who can have total bilirubin <3.0 mg/dL). Direct bilirubin ≤ULN for patients with total bilirubin levels >1.5× ULN
8. International Normalized Ratio (INR) or Prothrombin Time (PT) ≤1.5×ULN unless patient is receiving anticoagulant therapy as long as PT or PTT is within therapeutic range of intended use of anticoagulants
9. Activated Partial Thromboplastin Time (aPTT) ≤1.5×ULN unless patient is receiving anticoagulant therapy as long as PT or PTT is within therapeutic range of intended use of anticoagulants Female patients of childbearing potential must have a negative urine or serum pregnancy test within 72 hours prior to receiving the first dose of study medication. If the urine test is positive or cannot be confirmed as negative, a serum pregnancy test will be required. Female patients of childbearing potential must be willing to use an adequate method of contraception as outlined in Section 6.4.5.3, for the course of the study through 120 days after the last dose of study medication. Abstinence is acceptable if this is the usual lifestyle and preferred contraception for the patient. Male patients of childbearing potential must agree to use an adequate method of contraception as outlined in Section 6.4.5.3, starting with the first dose of study therapy through 120 days after the last dose of study therapy. Abstinence is acceptable if this is the usual lifestyle and preferred contraception for the patient

### Exclusion Criteria:

Is currently participating and receiving study therapy or has participated in a study of an investigational agent and received study therapy or used an investigational device within 4 weeks of first dose of treatment.
Received any systemic therapy for cancer treatment including immunotherapeutic agents such as anti-PD1 or anti-PD-L1 antibody therapy.
Has had a prior anti-cancer monoclonal antibody (mAb) within 4 weeks prior to study Day 1 or who has not recovered (ie, ≤Grade 1 or at baseline) from adverse events due to agents administered more than 4 weeks earlier.
Has had prior chemotherapy, targeted small molecule therapy, or radiation therapy within 30 days prior to study Day 1 or who has not recovered (i.e., ≤Grade 1 or at baseline) from adverse events due to a previously administered agent.
Subjects with ≤Grade 2 neuropathy or ≤Grade 2 alopecia are an exception to this criterion and can qualify for the study.
If subject received major surgery, they must have recovered adequately from the toxicity and/or complications from the intervention prior to starting therapy.
Patients cannot receive or have received any radiation therapy at the biopsy sites.
Received radiation therapy to the lung >30 Gy within 6 months of first dose of study treatment.
Received prior tyrosine kinase inhibitor therapy or palliative radiation within 7 days of first dose of study treatment.
Has known active central nervous system (CNS) metastases and/or carcinomatous meningitis.
Patients with previously treated brain metastases can participate provided they are stable (without evidence of progression by imaging (using the identical imaging modality for each assessment, either MRI or CT scan) for at least 4 weeks prior to the first dose of trial treatment and any neurologic symptoms have returned to baseline), have no evidence of new or enlarging brain metastases, and are not using steroids for at least 7 days prior to trial treatment. This exception does not include carcinomatous meningitis which is excluded regardless of clinical stability.
Patients cannot receive any non-oncology vaccine therapy during the period of NEO PV-01 or pembrolizumab administration and until at least 8 weeks after the last dose of the booster vaccine.
Seasonal influenza vaccines are allowed but cannot be administered between the first dose of pembrolizumab and the last booster dose of NEO-PV-01.
Has received transfusion of blood products (including platelets or red blood cells) or administration of colony stimulating factors (including G-CSF, GM-CSF or recombinant erythropoietin) within 4 weeks prior to study Day 1.
Has active autoimmune disease that has required systemic treatment in past 2 years (i.e., with use of disease modifying agents, corticosteroids or immunosuppressive drugs).
Replacement therapy (e.g., thyroxine, insulin, or physiologic corticosteroid replacement therapy for adrenal or pituitary insufficiency) is not considered a form of systemic treatment.
Has a diagnosis of immunodeficiency or is receiving chronic systemic steroid therapy (in dosing exceeding 10 mg daily of prednisone equivalent) or any other form of immunosuppressive therapy within 7 days prior to the first dose of trial treatment. The use of physiologic doses of corticosteroids can be approved after consultation with the Sponsor.
Received a live-virus vaccination within 30 days of planned treatment start.
Have symptomatic ascites or pleural effusion.
Has a history of (non-infectious) pneumonitis that required steroids or current pneumonitis.
Has a history of interstitial lung disease.
Has an active infection requiring systemic therapy.
Has a known history of Human Immunodeficiency Virus (HIV) (HIV 1/2 antibodies).
Has known active Hepatitis B (e.g., HBsAg reactive) or Hepatitis C (e.g., HCV RNA is detected).
Have an uncontrolled intercurrent illness including, but not limited to, ongoing or active infection requiring treatment, symptomatic congestive heart failure, unstable angina pectoris, cardiac arrhythmia.
Have any underlying medical condition, psychiatric condition, or social situation that, in the opinion of the Investigator, would compromise study administration as per protocol or compromise the assessment of AEs.
Have a planned major surgery.
Is pregnant or breastfeeding, or expecting to conceive or father children within the projected duration of the trial, starting with the screening visit through 120 days after the last dose of trial treatment.
Nursing women are excluded from this study because there is an unknown but potential risk of AEs in nursing infants secondary to treatment of the mother with pembrolizumab, personalized neoantigen peptides, and adjuvant.
Have a history of an invasive metastatic disease, except for the following:
1. Individuals with a history of invasive metastatic disease are eligible if they have been disease free for at least 2 years and are deemed by the Investigator to be at low risk for recurrence of that metastatic disease;
2. Exceptions include basal cell carcinoma of the skin, squamous cell carcinoma of the skin that has undergone potentially curative therapy or in situ cervical cancer. Patients with nonsquamous NSCLC having functionally significant anaplastic lymphoma kinase (ALK) translocations or epidermal growth factor receptor (EGFR) mutations who have not received prior treatment with ALK or EGFR inhibitor. Have severe hypersensitivity (≥Grade 3) to pembrolizumab and/or any of its excipients.

### IX. Additional Therapies

The tumor specific neoantigen peptides and pharmaceutical compositions described herein can also be administered in further combination with another agent, for example a therapeutic agent. In certain embodiments, the additional agents can be, but are not limited to, chemotherapeutic agents, anti-angiogenesis agents and agents that reduce immune-suppression.

The neoplasia vaccine or immunogenic composition and one or more inhibitors, such as a checkpoint inhibitor or chemotherapeutic agent, can be administered before, during, or after administration of the additional agent. In embodiments, the neoplasia vaccine or immunogenic composition and/or one or more inhibitors, such as a checkpoint inhibitor or chemotherapeutic agent, are administered before the first administration of the additional agent. In other embodiments, the neoplasia vaccine or immunogenic composition and/or one or more inhibitors, such as a checkpoint inhibitor or chemotherapeutic agent, are administered after the first administration of the additional therapeutic agent (e.g., 1, 2, 3, 4, 5, 6, 7, 8, 9, 10, 11, 12, 13, 14 or more days). In embodiments, the neoplasia vaccine or immunogenic composition and one or more inhibitors, such as a checkpoint inhibitor or chemotherapeutic agent, are administered simultaneously with the first administration of the additional therapeutic agent.

The therapeutic agent is for example, a chemotherapeutic or biotherapeutic agent, radiation, or immunotherapy. Any suitable therapeutic treatment for a particular cancer can be administered. Examples of chemotherapeutic and biotherapeutic agents include, but are not limited to, an angiogenesis inhibitor, such ashydroxy angiostatin K 1-3, DL-a-Difluoromethyl-omithine, endostatin, fumagillin, genistein, minocycline, staurosporine, and thalidomide; a DNA intercaltor/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-beiizotriazine 1,4-dioxide, Aminopterin, Cytosine β-D-arabinofuranoside, 5-Fluoro-5′˜deoxyuridine, 5-Flurorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-l-imidazole-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatins 15, Nocodazole, Paclitaxel, Podophyllotoxin, Rhizoxin, Vinblastine, Vincristine, Vindesine, and Vinorelbine (Navelbine); and an unclassified therapeutic agent, such as 17-(Allylamino)-1 7-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-α, Rapamycin, Sex hormone-binding globulin, Thapsigargin, and Urinary trypsin inhibitor fragment (Bikunin). The therapeutic agent can be altretamine, amifostine, asparaginase, capecitabine, cladribine, cisapride, cytarabine, dacarbazine (DT1C), dactinomycin, dronabinol, epoetin alpha, filgrastim, fludarabine, gemcitabine, granisetron, ifosfamide, irinotecan, lansoprazole, levamisole, leucovorin, megestrol, mesna, metoclopramide, mitotane, omeprazole, ondansetron, pilocarpine, prochloroperazine, or topotecan hydrochloride. The therapeutic agent can be a monoclonal antibody such as rituximab (Rituxan®), alemtuzumab (Campath®), Bevacizumab (Avastin®), Cetuximab (Erbitux®), panitumumab (Vectibix®), and trastuzumab (Herceptin®), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, 1311-tosit.umomab, ado-trastuzumab emtansine, lapatinib (Tykerb®), pertuzumab (Perjeta™), ado-trastuzumab emtansine (adcylam), regorafenib (Stivarga®), sunitinib (Sutent®), Denosumab (Xgeva®), sorafenib (Nexavar®), pazopanib (Votrient®), axitinib (Ini ta®), dasatinib (Sprycel®), nilotinib (Tasigna®), bosutinib (Bosulif®), ofatumumab (Arzerra®), obinutuzumab (Gazyva®), ibrutinib (Imbruvica®), idelalisib (Zydelig®), crizotinib (Xalkori®), erlotinib (Tarceva®), afatimb dimaleate (Giiotrif®), ceritinib (LDK378/Zykadia), Tositumomab and 1311-tositumomab (Bexxar®), ibritumomab tiuxetan (Zevalin®), brentuximab vedotin (Adcetris®), bortezomib (Velcade®), siltuximab (Sylvant™), trametinib (Mekinist®), dabrafenib (Tafmlar®), pembrolizimiab (Keytruda®), carfilzomib (Kyprolis®), Ramucirumab (Cyramza™), Cabozantinib (Cometriq™), vandetanib (Caprelsa®), Optionally, the therapeutic agent is a neoantigen. The therapeutic agent can be a cytokine such as interferons (INFs), interleukins (ILs), or hematopoietic growth factors. The therapeutic agent can be INF-α, IL-2, Aldesleukin, IL-2, Erythropoietin, Granulocyte-macrophage colony-stimulating factor (GM-CSF) or granulocyte colony-stimulating factor. The therapeutic agent can be a targeted therapy such as toremifene (Fareston®), fulvestrant (Faslodex®), anastrozole (Arimidex®), exemestane (Aromasin®), letrozole (Femara®), ziv-aflibercept (Zaltrap®), alitretinoin (Panretin®), temsirolimus (Torisel®), Tretinoin (Vesanoid®), denileukin diftitox (Ontak®), vorinostat (Zoiinza®), romidepsin (Istodax®), bexarotene (Targretin®), pralatrexate (Foiotyn®), lenaliomide (Revlimid®), belinostat (Beleodaq™), lenaliomide (Revlimid®), pomalidomide (Pomalyst®), Cabazitaxel (Jevtana®), enzaluiamide (Xtandi®), abiraterone acetate (Zytiga®), radium 223 chloride (Xofigo®), or everolimus (Afinitor®). Additionally, the therapeutic agent can be an epigenetic targeted drug such as HDAC inhibitors, kinase inhibitors, DNA methyltransferase inhibitors, histone demethylase inhibitors, or histone methylation inhibitors. The epigenetic drugs can be Azacitidine (Vidaza), Decitabine (Dacogen), Vorinostat (Zoiinza), Romidepsin (Istodax), or Ruxolitinib (Jakafi). For prostate cancer treatment, a chemotherapeutic agent with which anti-CTLA-4 can be combined is paclitaxel (TAXOL).

In certain embodiments, the one or more additional agents are one or more anti-glucocorticoid-induced tumor necrosis factor family receptor (GITR) agonistic antibodies. GITR is a costimulatory molecule for T lymphocytes, modulates innate and adaptive immune system and has been found to participate in a variety of immune responses and inflammatory processes. GITR was originally described by Nocentini et al. after being cloned from dexamethasone-treated murine T cell hybridomas (Nocentini et al, Proc Natl Acad Sci USA 94:6216-6221.1997). Unlike CD28 and CTLA-4, GITR has a very low basal expression on naive CD4+ and CD8+ T cells (Ronchetti et al. Eur J Immunol 34:613-622.2004). The observation that GITR stimulation has immunostimulatory effects in vitro and induced autoimmunity in vivo prompted the investigation, of the antitumor potency of triggering this pathway. A review of Modulation Of CTLA4 And GITR For Cancer Immunotherapy can be found in Cancer Immunology and Immunotherapy (Avogadri et al. Current Topics in Microbiology and Immunology 344. 2011). Other agents that can contribute to relief of immune suppression include checkpoint inhibitors targeted at another member of the CD28/CTLA4 Ig superfamily such as BTLA, LAG 3, ICOS, PDL1 or J (Page et a, Annual Review of Medicine 65:27 (2014)).

In certain embodiments, the one or more additional agents are synergistic in that they increase immunogenicity after treatment. In one embodiment the additional agent allows for lower toxicity and/or lower discomfort due to lower doses of the additional therapeutic agents or any components of the combination therapy described herein. In another embodiment the additional agent results in longer lifespan due to increased effectiveness of the combination therapy described herein. Chemotherapeutic treatments that enhance the immunological response in a patient have been reviewed (Zitvogel et al., Immunological aspects of cancer chemotherapy. Nat Rev Immunol. 2008 January; 8(1):59-73). Additionally, chemotherapeutic agents can be administered safely with immunotherapy without inhibiting vaccine specific T-cell responses (Perez et al., A new era in anticancer peptide vaccines. Cancer Can 2010). In one embodiment the additional agent is administered to increase the efficacy of the combination therapy described herein. In one embodiment the additional agent is a chemotherapy treatment. In one embodiment low doses of chemotherapy potentiate delayed-type hypersensitivity (DTH) responses. In one embodiment the chemotherapy agent targets regulatory T-cells. In one embodiment cyclophosphamide is the therapeutic agent. In one embodiment cyclophosphamide is administered prior to vaccination. In one embodiment cyclophosphamide is administered as a single dose before vaccination (Walter et al, Multipeptide immune response to cancer vaccine IMA901 after single-dose cyclophosphamide associates with longer patient survival. Nature Medicine; 18:8 2012). In another embodiment cyclophosphamide is administered according to a metronomic program, where a daily dose is administered for one month (Ghiringhelli et al, Metronomic cyclophosphamide regimen selectively depletes CD4+CD25+ regulatory T cells and restores T and NK effector functions in end stage cancer patients. Cancer Immunol Immunother 2007 56:641-648). In another embodiment taxanes are administered before vaccination to enhance T-cell and NK-cell functions (Zitvogel et al, 2008). In another embodiment a low dose of a chemotherapeutic agent is administered with the combination therapy described herein. In one embodiment the chemotherapeutic agent is estramustine. In one embodiment the cancer is hormone resistant prostate cancer. A >50% decrease in serum prostate specific antigen (PSA) was seen in 8.7% of advanced hormone refractory prostate cancer patients by personalized vaccination alone, whereas such a decrease was seen in 54% of patients when the personalized vaccination, was combined with a low dose of estramustine (I ton et al., Personalized peptide vaccines: A new therapeutic modality for cancer. Cancer Sci 2006; 97: 970-976). In another embodiment glucocorticoids are not administered with or before the combination therapy described herein (Zitvogef et al., 2008). In another embodiment glucocorticoids are administered after the combination therapy described herein. In another embodiment Gemcitabine is administered before, simultaneously, or after the combination therapy described herein to enhance the frequency of tumor specific CTL precursors (Zitvogel et al., 2008). In another embodiment 5-ftuorouracil is administered with the combination therapy described herein as synergistic effects were seen with a peptide based vaccine (Zitvogel et al., 2008). In another embodiment an inhibitor of Braf, such as Vemurafenib, is used as an additional agent. Braf inhibition has been shown to be associated with an increase in melanoma antigen expression and T-cell infiltrate and a decrease in immunosuppressive cytokines in tumors of treated patients (Frederick et al, BRAF inhibition is associated with enhanced melanoma antigen expression and a more favorable tumor microenvironment in patients with metastatic melanoma. Clin Cancer Res. 2013; 19:1225-1231). In another embodiment an inhibitor of tyrosine kinases is used as an additional agent. In one embodiment the tyrosine kinase inhibitor is used before vaccination with the combination therapy described herein. In one embodiment the tyrosine kinase inhibitor is used simultaneously with the combination therapy described herein. In another embodiment the tyrosine kinase inhibitor is used to create a more immune permissive environment. In another embodiment the tyrosine kinase inhibitor is sunitinib or matinib mesylate. It has previously been shown that favorable outcomes could be achieved with sequential administration of continuous daily dosing of sunitinib and recombinant vaccine (Farsaci et al., Consequence of dose scheduling of sunitinib on host immune response elements and vaccine combination therapy. Int J Cancer; 130: 1948-1959). Sunitinib has also been shown to reverse type-1 immune suppression using a daily dose of 50 mg/day (Finke et al., Sunitinib Reverses Type-1 Immune Suppression and Decreases T-Regulatory Cells in Renal Cell Carcinoma Patients. Clin Cancer Res 2008; 14(20)). In another embodiment targeted therapies are administered in combination with the combination therapy described herein. Doses of targeted therapies have been described previously (Alvarez, Present and future evolution of advanced breast cancer therapy. Breast Cancer Research 2010, 12 (Suppl 2):S1). In another embodiment temozolomide is administered with the combination therapy described herein. In one embodiment temozolomide is administered at 200 mg day for 5 days every fourth week of a combination therapy with the combination therapy described herein. Results of a similar strategy have been shown to have low toxicity (Kyte et al., Telomerase Peptide Vaccination Combined with Temozolomide: A Clinical Trial in Stage IV Melanoma Patients. Clin Cancer Res; 17(13) 2011). In another embodiment the combination therapy is administered with an additional therapeutic agent that results in lymphopenia. In one embodiment the additional agent is temozolomide. An immune response can still be induced under these conditions (Sampson et al., Greater chemotherapy-induced lymphopenia enhances tumor-specific immune responses that eliminate EGFRvIII-expressing tumor cells in patients with glioblastoma. Neuro-Oncology 13(3):324-333, 2011).

The herein-described compositions and methods can be used on patients in need thereof with any cancer according to the general flow process comprising subject identification, collecting informed consent and pre-screening the patients. Patients can then undergo an assessment of the specific type of cancer and the mutations causing the cancer. The nucleic acid material collected from a patient can be used for exome sequencing (tumor and normal tissue), RNA sequencing for the preparation of specific personalized vaccine. Patients in need thereof can receive a series of priming vaccinations with a mixture of personalized tumor-specific peptides. Additionally, over a 4 week period the priming can be followed by two boosts during a maintenance phase. All vaccinations are subcutaneously delivered. The vaccine or immunogenic composition is evaluated for safety, tolerability, immune response and clinical effect in patients and for feasibility of producing vaccine or immunogenic composition and successfully initiating vaccination within an appropriate time frame. The first cohort can consist of 5 patients, and after safety is adequately demonstrated, an additional cohort of 10 patients can be enrolled. Peripheral blood is extensively monitored for peptide-specific T-cell responses and patients are followed for up to two years to assess disease recurrence.

### X. Administering Combination Therapy Consistent with Standard of Care

In another aspect, the combination therapy described herein provides selecting the appropriate point to administer the combination therapy in relation to and within the standard of care for the cancer being treated for a patient in need thereof. The studies described herein show that the combination therapy can be effectively administered even within the standard of care that includes surgery, radiation, or chemotherapy. The standards of care for the most common cancers can be found on the website of National Cancer Institute (http://www.cancer.gov/eancertopies). The standard of care is the current treatment that is accepted by medical experts as a proper treatment for a certain type of disease and that is widely used by healthcare professionals. Standard or care is also called best practice, standard medical care, and standard therapy. Standards of Care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CAILs), and adoptive T-cell therapy. The combination therapy described herein can be incorporated within the standard of care. The combination therapy described herein can also be administered where the standard of care has changed due to advances in medicine.

Incorporation of the combination therapy described herein can depend on a treatment step in the standard of care that can lead to activation of the immune system. Treatment steps that can activate and function synergistically with the combination therapy have been described herein. The therapy can be advantageously administered simultaneously or after a treatment that activates the immune system.

Incorporation of the combination therapy described herein can depend on a treatment step in the standard of care that causes the immune system to be suppressed. Such treatment steps can include irradiation, high doses of alkylating agents and/or methotrexate, steroids such as glucosteroids, surgery, such as to remove the lymph nodes, imatinib mesylate, high doses of TNF, and taxanes (Zitvogel et al., 2008). The combination therapy can be administered before such steps or can be administered after.

In one embodiment the combination therapy can be administered after bone marrow transplants and peripheral blood stem cell transplantation. Bone marrow transplantation and peripheral blood stem cell transplantation are procedures that restore stem cells that were destroyed by high doses of chemotherapy and/or radiation therapy. After being treated with high-dose anticancer drugs and/or radiation, the patient receives harvested stem cells, which travel to the bone marrow and begin to produce new blood cells. A “mini-transplant” uses lower, less toxic doses of chemotherapy and/or radiation to prepare the patient for transplant. A “tandem transplant” involves two sequential courses of high-dose chemotherapy and stem cell transplant. In autologous transplants, patients receive their own stem cells. In syngeneic transplants, patients receive stem cells from their identical twin. In allogeneic transplants, patients receive stem cells from their brother, sister, or parent. A person who is not related to the patient (an unrelated donor) also can be used, In some types of leukemia, the graft-versus-tumor (GVT) effect that occurs after allogeneic BMT and PBSCT is crucial to the effectiveness of the treatment. GVT occurs when white blood cells from the donor (the graft) identify the cancer cells that remain in the patient's body after the chemotherapy and/or radiation therapy (the tumor) as foreign and attack them. Immunotherapy with the combination therapy described herein can take advantage of this by vaccinating after a transplant. Additionally, the transferred cells can be presented with neoantigens of the combination therapy described herein before transplantation.

In one embodiment the combination therapy is administered to a patient in need thereof with a cancer that requires surgery. In one embodiment the combination therapy described herein is administered to a patient in need thereof in a cancer where the standard of care is primarily surgery followed by treatment to remove possible micro-metastases, such as breast cancer. Breast cancer is commonly treated by various combinations of surgery, radiation therapy, chemotherapy, and hormone therapy based on the stage and grade of the cancer. Adjuvant therapy for breast cancer is any treatment given after primary therapy to increase the chance of long-term survival. Neoadjuvant therapy is treatment given before primary therapy. Adjuvant therapy for breast cancer is any treatment given after primary therapy to increase the chance of long-term disease-free survival. Primary therapy is the main treatment used to reduce or eliminate the cancer. Primary therapy for breast cancer usually includes surgery, a mastectomy (removal of the breast) or a lumpectomy (surgery to remove the tumor and a small amount of normal tissue around it; a type of breast-conserving surgery). During either type of surgery, one or more nearby lymph nodes are also removed to see if cancer cells have spread to the lymphatic system. When a woman has breast-conserving surgery, primary therapy almost always includes radiation therapy. Even in early-stage breast cancer, cells can break away from the primary tumor and spread to other parts of the body (metastasize). Therefore, doctors give adjuvant therapy to kill any cancer cells that can have spread, even if they cannot be detected by imaging or laboratory tests.

In one embodiment the combination therapy is administered consistent with the standard of care for Ductal carcinoma in situ (DCIS). Care for this breast cancer type comprises: 1. Breast-conserving surgery and radiation therapy with or without tamoxifen. 2. Total mastectomy with or without tamoxifen. 3. Breast-conserving surgery without radiation therapy.

The combination therapy can be administered before breast conserving surgery or total mastectomy to shrink the tumor before surgery. In another embodiment the combination therapy can be administered as an adjuvant therapy to remove any remaining cancer cells.

In another embodiment patients diagnosed with stage I, II, IIIA, and Operable IIIC breast cancer are treated with the combination therapy as described herein. Care for this breast cancer type comprises:

1. Local-regional treatment;
Breast-conserving therapy (lumpectomy, breast radiation, and surgical staging of the axilla).
Modified radical mastectomy (removal of the entire breast with level I-II axillary dissection) with or without breast reconstruction.
Sentinel node biopsy.
   2. Adjuvant radiation therapy postmastectomy in axillary node-positive tumors:
For one to three nodes: unclear role for regional radiation (infra/supraclavicular nodes, internal mammary nodes, axillary nodes, and chest wall).
For more than four nodes or extranodal involvement: regional radiation is advised.
   3. Adjuvant systemic therapy

In one embodiment the combination therapy is administered as a neoadjuvant therapy to shrink the tumor. In another embodiment the combination is administered as an adjuvant systemic therapy.

In another embodiment patients diagnosed with inoperable stage IIIB or IIIC or inflammatory breast cancer are treated with the combination therapy as described herein. The standard of care for this breast cancer type is: 1. Multimodality therapy delivered with curative intent is the standard of care for patients with clinical stage IHB disease. 2. Initial surgery is generally limited to biopsy to permit the determination of histology, estrogen-receptor (ER) and progesterone-receptor (PR) levels, and human epidermal growth factor receptor 2 (HER2/neu) overexpression. Initial treatment with anthracycline-based chemotherapy and/or taxane-based therapy is standard. For patients who respond to neoadjuvant chemotherapy, local therapy can consist of total mastectomy with axillary lymph node dissection followed by postoperative radiation therapy to the chest wall and regional lymphatics. Breast-conserving therapy can be considered in patients with a good partial or complete response to neoadjuvant chemotherapy. Subsequent systemic therapy can consist of further chemotherapy. Hormone therapy should be administered to patients whose tumors are ER-positive or unknown. All patients should be considered candidates for clinical trials to evaluate the most appropriate fashion in which to administer the various components of multimodality regimens.

In one embodiment the combination therapy is administered as part of the various components of multimodality regimens. In another embodiment the combination therapy is administered before, simultaneously with, or after the multimodality regimens. In another embodiment the combination therapy is administered based on synergism between the modalities. In another embodiment the combination therapy is administered after treatment with anthracycline-based chemotherapy and/or taxane-based therapy (Zirvogel et al, 2008). Treatment after administering the combination therapy can negatively affect dividing effector T-cells. The combination therapy can also be administered after radiation.

In another embodiment the combination therapy described herein is used in the treatment in a cancer where the standard of care is primarily not surgery and is primarily based on systemic treatments, such as Chronic Lymphocytic Leukemia (CLL).

### XI. Vaccine or Immunogenic Composition Kits and Co-Packaging

In an aspect, the disclosure provides kits containing any one or more of the elements discussed herein to allow administration of the combination therapy. Elements can be provided individually or in combinations, and can be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents can be provided in any suitable container. For example, a kit can provide one or more delivery or storage buffers. Reagents can be provided in a form that is usable in a particular process, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more of the vectors, proteins and/or one or more of the polynucleotides described herein. The kit can advantageously al low the provision of all elements of the systems of the disclosure. Kits can involve vector(s) and/or particle(s) and/or nanoparticle(s) containing or encoding RNA(s) for 1-50 or more neoantigen mutations to be administered to an animal, mammal, primate, rodent, etc., with such a kit including instructions for administering to such a eukaryote; and such a kit can optionally include any of the anti-cancer agents described herein. The kit can include any of the components above (e.g., vector(s) and/or particle(s) and/or nanoparticle(s) containing or encoding RNA(s) for 1-50 or more neoantigen mutations, neoantigen proteins or peptides, checkpoint inhibitors, or platinum based chemotherapeutic agent) as well as instructions for use with any of the methods of the present disclosure.

In one embodiment the kit contains at least one vial with an immunogenic composition or vaccine and at least one vial with an anticancer agent. In one embodiment kits can comprise ready to use components that are mixed and ready to administer. In one aspect a kit contains a ready to use immunogenic or vaccine composition and a ready to use anti-cancer agent. The ready to use immunogenic or vaccine composition can comprise separate vials containing different pools of immunogenic compositions. The immunogenic compositions can comprise one vial containing a viral vector or DNA plasmid and the other vial can comprise immunogenic protein. The ready to use anticancer agent can comprise a cocktail of anticancer agents or a single anticancer agent. Separate vials can contain different anti-cancer agents. In another embodiment a kit can contain a ready to use anti-cancer agent and an immunogenic composition or vaccine in a ready to be reconstituted form. The immunogenic or vaccine composition can be freeze dried or lyophilized. The kit can comprise a separate vial with a reconstitution buffer that can be added to the lyophilized composition so that it is ready to be administered. The buffer can advantageously comprise an adjuvant or emulsion according to the present disclosure. In another embodiment the kit can comprise a ready to reconstitute anticancer agent and a ready to reconstitute immunogenic composition or vaccine. In this aspect both can be lyophilized. In this aspect separate reconstitution buffers for each can be included in the kit. The buffer can advantageously comprise an adjuvant or emulsion according to the present disclosure. In another embodiment the kit can comprise single vials containing a dose of immunogenic composition and anti-cancer agent that are administered together. In another aspect multiple vials are included so that one vial is administered according to a treatment timeline. One vial can only contain the anti-cancer agent for one dose of treatment, another can contain both the anti-cancer agent and immunogenic composition for another dose of treatment and one vial can only contain the immunogenic composition for yet another dose. In a further aspect the vials are labeled for their proper administration to a patient in need thereof. The immunogen or anti-cancer agents of any embodiment can be in a lyophilized form, a dried form or in aqueous solution as described herein. The immunogen can be a live attenuated virus, protein, or nucleic acid as described herein.

In one embodiment the anticancer agent is one that enhances the immune system to enhance the effectiveness of the immunogenic composition or vaccine. In an embodiment the anti-cancer agent is an inhibitor, such as a checkpoint inhibitor or chemotherapeutic agent. In another embodiment the kit contains multiple vials of immunogenic compositions and anti-cancer agents to be administered at different time intervals along a treatment plan. In another embodiment the kit can comprise separate vials for an immunogenic composition for use in priming an immune response and another immunogenic composition to be used for boosting. In one aspect the priming immunogenic composition could be DNA or a “viral, vector and the boosting immunogenic composition can be protein. Either composition can be lyophilized or ready for administering. In another embodiment different cocktails of anti-cancer agents containing at least one anticancer agent are included in different vials for administration in a treatment plan.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### Example 1: Personalized Neoantigen Therapy NEO-PV-01 in Combination with Chemotherapy and Anti-PD-1 in the Treatment of First-Line Non-Squamous NSCLC

I. Treatment with NEO-PV-01 Plus Chemotherapy and Anti-PD-1 is Feasible and Safe in First-Line NSCLC

This study reports data from a phase 1b clinical trial combining a personalized neoantigen vaccine (NEO-PV-01), pemetrexed, carboplatin and pembrolizumab in first-line metastatic non-squamous NSCLC. In addition to demonstrating that this treatment regimen was safe and well-tolerated, detailed molecular and immune analyses were performed of both the tumor microenvironment (TME) and peripheral blood from these patients. Multiple factors in the TME were correlated with increased progression-free survival (PFS), including increased T cell infiltration, increased MHC Class II expression on immune cells, and increased diversity of the T cell receptor (TCR) repertoire. Very few pre-existing neoantigen-specific immune responses were observed, but ex vivo responses to peptides used in the vaccine post-vaccination were detected in all of the 13 patients tested. Epitope spread to non-vaccinating epitopes, including mutant KRAS epitopes, was also observed in the majority of patients. Immunophenotyping revealed T cell responses composed of both CD4+ and CD8+ responses with cytotoxic potential. Single cell sequencing of neoantigen-specific CD4+ T cell responses in the periphery revealed a strong effector, proliferative and cytotoxic phenotype. The analyses presented here suggest a robust effect of peptide vaccination in combination with chemotherapy and anti-PD-1, particularly on CD4+ T cells, supporting further investigation of this treatment in additional clinical trials.

Study Design: The study was designed as a single arm study to assess the safety and immunogenicity of the personalized vaccine NEO-PV-01 in combination with chemotherapy and pembrolizumab in first line non-squamous NSCLC. Patients were enrolled at four clinical sites in the United States. The protocol specified no prior systemic treatment for metastatic disease and no prior immunotherapy with anti-PD-1 or PD-L1 antibodies. Tumor PD-L1 status was assessed for all patients, but enrollment was not restricted based on PD-L1 status (Methods). Major study endpoints included evaluations for safety, objective response rate (ORR), progression free survival (PFS), overall survival (OS) and comprehensive immune analysis in blood and tumor (Methods, study protocol).

Production of the personalized neoantigen vaccine NEO-PV-01 was performed as previously described (Ott et al 2020). Briefly, tumor mutations were first identified by whole exome and RNA sequencing of each patient's formalin-fixed tumor and matched normal cells from blood as outlined in the vaccine manufacturing schema (FIG. 1A). Epitopes were then selected based on bioinformatic algorithms as previously described in (Ott et al., 2020) and incorporated into peptide based neoantigen vaccines. (Nielsen and Andreatta, 2016; Abelin et al., 2017). Each neoantigen vaccine consisted of up to 20 unique peptides that were formulated in up to four distinct pools with adjuvant poly-ICLC (polyinosinic-polycytidylic acid stabilized with polylysine and caboxymethylcellulaose). Patients received a combination of pembrolizumab, pemetrexed and carboplatin administered every three weeks (a cycle) for four cycles during vaccine production. Pemetrexed maintenance therapy was not administered in this study. Following this initial triplet therapy, starting at week 12, NEO-PV-01 was administered intramuscularly in four separate anatomical locations. As reported previously (Ott et al., 2020), five priming and two booster doses of the neoantigen vaccine were given over a period of the next 12 weeks. Pembrolizumab was continued both during the vaccine and post-vaccine time periods up to two years in the absence of unacceptable toxicities or disease progression (FIG. 1B).

38 patients were enrolled and received at least one dose of pembrolizumab and were defined as the intention-to-treat (ITF) set (FIG. 1C). Among these patients, 3 (8%) had prior therapy with 2 of the 3 patients receiving cisplatin plus pemetrexed for 3 months as an adjuvant regimen. The third patient had three prior therapies that included carboplatin plus paclitaxel for 3 months, followed by erlotinib for 2 years, and a vaccine therapy (alpha1,3 galactosyl transferase) for 4 months as adjuvant regimens. Most of the patients (87%) on our study presented with stage IV disease (Table 1). Seventeen of the 38 patients (45%) in the ITT set were not vaccinated due to early study termination for reasons including inability to manufacture vaccine due to inadequate tumor and/or sufficient number of neoantigens (10 patients), adverse events (2 patients), patient consent withdrawn (2 patients), progressive disease, an investigator decision, or administration of a study-prohibited concomitant medication (1 patient each). Therefore, the vaccinated patient subset (VAX) included 21 patients, of which 16 completed vaccination (Full VAX) with five discontinuing treatment due to PD during the vaccine regimen (FIG. 1C).

TABLE 1
Patient demographics and baseline disease characteristics

| | ITT | VAX |
|---|---|---|
| | (N = 38) | (N = 21) |
| Median Age (years) | 62.5 (56-73) | 63 (56-69) |
| Sex M/F, n (%) | 15 (39%) / 23 (61%) | 7 (33%) / 14 (67%) |
| Prior therapy, n (%) | 3 (8%) | 0% |
| Stage IV, n (9%) | 33 (87%) | 21 (100%) |
| Median TMB (range) | 141 (2, 776) | 279 (96, 776) |
| Smokers (current or prior), % | 82% | 95% |
| Tumor PD-L1 expression, | | |
| n (%)* | | |
| <1% | 11 (42%) | 9 (43%) |
| ≥1%-<50% | 12 (46%) | 10 (47%) |
| ≥50% | 3 (12%) | 2 (10%) |
| Median sum of target | 8.6 | 8.3 |
| lesions (cm) | | |
| ECOG 0/1, n (%) | 11 (29%) / 27 (71%) | 7 (33%) / 14 (67%) |

*TMB is represented as count of non-silent mutations detected in exonic regions
** Tumor PD-L1 expression NA for 12 of 38 ITT patients

Analysis of the mutational status for common mutations observed in NSCLC among the ITT patient set of 38 patients revealed 19 patients with KRAS mutations, 15 patients with TP53 mutations, 6 patients with KEAP1 mutations, with no patients having mutations in STK11 (Table S1). Depending on the patient's HLA alleles and predicted binding affinity of the driver mutants, these common mutations were either included as part of the vaccine or were monitored for epitope spread analysis (Table S1). A TP53 and a KEAP1 mutation were included in the vaccine for the corresponding patients. The reasons that driver mutations were not included in the vaccine or monitored for epitope spread were either very low immunogenicity scores as defined by our prediction pipeline or the predicted immunogenic peptides had poor solubility and failed the manufacturing process.

TABLE S1
Driver mutation status for ITT set. KRAS, TP53 and KEAPI mutation status
information for study patients. Information on whether the driver mutation was included in the vaccine
or epitope spread analysis is indicated, as well as if a post-vaccination immune response was observed
in either case. Y = yes, N = no.

| | | | | | Included | |
|---|---|---|---|---|---|---|
| | | | | | in Epitope | Post- |
| | | | | Included | Spread | vaccination |
| Patient | | | | in Vaccine | Analysis | Response |
| ID | Gene | Genome Change | Protein Change | (Y/N) | (Y/N) | (Y/N) |
| 2L1 | KRAS | chr12: 25398284_C > A | 12: G > V | N | N | N |
| 2L2 | KRAS | chr12: 25398285_C > A | 12: G > C | N | Y | Y |
| 2L3 | TP53 | chr17: 7577142_C > A | 266: GRNSFEVRVCAC | N | N | N |
| | | | PGRDRRTEEENLRKK | | | |
| | | | GEPHHELPPGSTKRA | | | |
| | | | LPNNTSSSPQPKKKPL | | | |
| | | | DGEYFTLQIRGRERF | | | |
| | | | EMFRELNEALELKDA | | | |
| | | | QAGKEPGGSRAHSSH | | | |
| | | | LKSKKGQSTSRHKKL | | | |
| | | | MFKTEGPDSD (SEQ | | | |
| | | | ID NO: 2)>. | | | |
| 2L4 | KRAS | chr12: 25380276_T > A | 61: Q > L | N | N | N |
| 2NVX1 | TP53 | chr17: 7578265_A > G | 195: I > T | N | N | N |
| 2L7 | KRAS | chr12: 25398285_C > A | 12: G > C | N | N | N |
| 2L8 | KEAP1 | chr19: 10602446_C > A | 378: G > C | N | Y | N |
| 2L8 | KRAS | chr12: 25398284_C > T | 12: G > D | N | N | N |
| 2NVX2 | KRAS | chr12: 25398284_C > T | 12: G > D | N | N | N |
| 2L9 | KRAS | chr12: 25398284_C > G | 12: G > A | N | Y | N |
| 2L16 | TP53 | chr17: 7578211_C > A | 213: R > L | N | Y | N |
| 2L16 | TP53 | chr17: 7578211_C > A | 213: R > L | Y | N | N |
| 2NVX3 | KRAS | chr12: 25398284_C > T | 12: G > D | N | N | N |
| 2UV6 | KRAS | chr12: 25398285_C > A | 12: G > C | N | N | N |
| 2UV3 | KEAP1 | chr19: 10602763_C > A | 272: R > L | N | N | N |
| 2UV3 | TP53 | chr17: 7578442_T > C | 163: Y > C | N | N | N |
| 2NVX4 | TP53 | chr17: 7578457_C > A | 158: R > L | N | N | N |
| 2L5 | KRAS | chr12: 25398284_C > A | 12: G > V | N | Y | Y |
| 2L5 | TP53 | chr17: 7577120_C > A | 273: R > L | N | N | N |
| 2NVX5 | KRAS | chr12: 25398284_C > T | 12: G > D | N | N | N |
| 2NVX5 | TP53 | chr17: 7577559_G > A | 241: S > F | N | N | N |
| 2L6 | KRAS | chr12: 25398285_C > A | 12: G > C | N | Y | N |
| 2NVX6 | KRAS | chr12: 25398284_C > G | 12: G > A | N | N | N |
| 2NVX6 | TP53 | chr17: 7578205_C > A | 215: S > I | N | N | N |
| 2NVX7 | TP53 | chr17: 7577539_G > A | 248: R > W | N | N | N |
| 2UV1 | KEAP1 | chr19: 10602646_T > G | 311: H > P | Y | N | N |
| 2UV1 | KRAS | chr12: 25398285_C > A | 12: G > C | N | Y | Y |
| 2UV1 | TP53 | chr17: 7578469_C > A | 154: G > V | N | Y | N |
| 2L11 | KRAS | chr12: 25398284_C > G | 12: G > A | N | N | N |
| 2L11 | TP53 | chr17: 7577559_G > T | 241: S > Y | N | N | N |
| 2NVX11 | KEAP1 | chr19: 10600381_G > A | 492: P > S | N | N | N |
| 2NVX11 | KRAS | chr12: 25398285_C > G | 12: G > R | N | N | N |
| 2L14 | TP53 | chr17: 7578188_C > A | 221: EPPEVGSDCTTIH | N | N | N |
| | | | YNYMCNSSCMGGM | | | |
| | | | NRRPILTIITLEDSSGN | | | |
| | | | LLGRNSFEVRVCACP | | | |
| | | | GRDRRTEEENLRKKG | | | |
| | | | EPHHELPPGSTKRAL | | | |
| | | | PNNTSSSPQPKKKPL | | | |
| | | | DGEYFTLQIRGRERF | | | |
| | | | EMFRELNEALELKDA | | | |
| | | | QAGKEPGGSRAHSSH | | | |
| | | | LKSKKGQSTSRHKKL | | | |
| | | | MFKTEGPDSD (SEQ | | | |
| | | | ID NO: 3)>. | | | |
| 2L15 | KEAP1 | chr19: 10600004_G > GC | 525: YDGQDQLNSVER | EN | Y | N |
| | | | YDVETETWTFVAPM | | | |
| | | | KHRRSALGITVHQGR | | | |
| | | | IYVLGGYDGHTFLDS | | | |
| | | | VECYDPDTDTWSEV | | | |
| | | | TRMTSGRSGVGVAV | | | |
| | | | TMEPCRKQIDQQNCT | | | |
| | | | C (SEQ ID NO: 4) > L | | | |
| 2L15 | KRAS | chr12: 25398285_C > A | 12: G > C | N | Y | Y |
| 2L13 | KRAS | chr12: 25398284_C > A | 12: G > V | N | Y | N |
| 2L13 | TP53 | chr17: 7578272_G > A | 193: H > Y | N | Y | N |
| 2UV5 | KEAP1 | chr19: 10602353_TG > T | 409: MSVPRNRIGVGV | N | N | N |
| | | | IDGHIYAVGGSHGCI | | | |
| | | | HHNSVERYEPERDE | | | |
| | | | WHLVAPMLTRRIGV | | | |
| | | | GVAVLNRLLYAVGG | | | |
| | | | FDGTNRLNSAECYYP | | | |
| | | | ERNEWRMITAMNTIR | | | |
| | | | SGAGVCVLHNCIYAA | | | |
| | | | GGYDGQDQLNSVER | | | |
| | | | YDVETETWTFVAPM | | | |
| | | | KHRRSALGITVHQGR | | | |
| | | | IYVLGGYDGHTFLDS | | | |
| | | | VECYDPDTDTWSEV | | | |
| | | | TRMTSGRSGVGVAV | | | |
| | | | TMEPCRKQIDQQNCT | | | |
| | | | C (SEQ ID NO: 5)>. | | | |
| 2UV5 | TP53 | chr17: 7578442_T > C | 163: Y > C | N | N | N |
| 2NVX8 | TP53 | chr17: 7578406_C > T | 175: R > H | N | N | N |
| 2L12 | KRAS | chr12: 25398284_C > A | 12: G > V | N | N | N |

The primary objective of the study was to evaluate the safety and tolerability of NEO-PV-01 in combination with pembrolizumab, pemetrexed and carboplatin (Tables S2, S3 and S4). The regimen was well-tolerated, consistent with the pembrolizumab plus pemetrexed/carboplatin safety profile. The most frequent related adverse events included nausea, emesis, diarrhea, fatigue, neutropenia, and anemia. The only related AE with clear increased incidence in the VAX group were injection site reactions, with 6 patients (29%) experiencing transient, low-grade events. Treatment related SAEs were uncommon, reported in 5 patients, only 1 of whom was in the VAX group [<5%]). Treatment discontinuations due to related AEs were also uncommon (2 patients, 5.3%) and no grade 5 events were observed. In summary, NEO-PV-01 in combination with pembrolizumab, pemetrexed, and carboplatin was safe and well-tolerated, with a safety profile similar to that previously reported in metastatic NSCLC (Ott et al., 2020).

TABLE S2
NT002 Safety Profile

| | | | Post- |
|---|---|---|---|
| | | Pre- | Vaccine |
| | All | Vaccination | Initiation |
| | Periods(ITT | (ITT Set, | (Vaccinated |
| | Set, N = 38) | N = 38) | Set, N = 21) |
| Adverse events (all) | | | |
| Averse Events | 37 (97.4) | 37 (97.4) | 20 (95.2) |
| Serious | 15 (39.5) | 11 (28.9) | 6 (28.6) |
| Grade ≥3 | 21 (55.3) | 18 (47.4) | 8 (38.1) |
| Leading to any study drug | 5 (13.2) | 3 (7.9) | 2 (9.5) |
| discontinuation | | | |
| Adverse events related to | | | |
| either NEO-PV-1 and/or | | | |
| other study drugs | | | |
| Averse Events | 33 (86.8) | 32 (84.2) | 17 (81.0) |
| Serious | 4 (10.5) | 3 (7.9) | 1 (4.8) |
| Grade ≥3 | 7 (18.4) | 7 (18.4) | 1 (4.8) |
| Leading to any study drug | 2 (5.3) | 2 (5.3) | 0 |
| discontinuation | | | |

Overview of numbers and percentages of patients reporting adverse events since first dose of study drug (All Periods), between first dose of study drug and first dose of vaccine (Pre-Vaccination), after first dose of vaccine to end of data reporting (Post-Vaccine Initiation).

TABLE S3
Treatment-related adverse events.

| | All Periods | | | Pre-Vaccination | | | Post-Vaccine Initiation | | |
|---|---|---|---|---|---|---|---|---|---|
| | (ITT Set, | | | (ITT Set, | | | (Vaccinated Set, | | |
| | N = 38) | | | N = 38) | | | N = 21) | | |
| | All | | Grade ≥3 | All | | Grade ≥3 | All | | Grade ≥3 |
| Fatigue | 13 | (34.2) | 0 | 10 | (26.3) | 0 | 6 | (28.6) | 0 |
| Injection site reaction | 6 | (15.8) | 0 | 0 | | 0 | 6 | (28.6) | 0 |
| Decreased appetite | 7 | (18.4) | 0 | 4 | (10.5) | 0 | 3 | (14.3) | 0 |
| Diarrhoea | 7 | (18.4) | 0 | 5 | (13.2) | 0 | 3 | (14.3) | 0 |
| Dysgeusia | 5 | (13.2) | 0 | 2 | (5.3) | 0 | 3 | (14.3) | 0 |

| Injection site | 3 | (7.9) | 0 | 0 | 0 | 3 | (14.3) | 0 |
|---|---|---|---|---|---|---|---|---|
| erythema | | | | | | | | |
| Injection site pain | 3 | (7.9) | 0 | 0 | 0 | 3 | (14.3) | 0 |
| Injection site pruritus | 3 | (7.9) | 0 | 0 | 0 | 3 | (14.3) | 0 |

Numbers and percentages of patients with adverse events since first dose of study drug that occurred in ≥10% of vaccine-initiated patients and were considered possibly or probably related to chemotherapy, pembrolizumab and/or vaccine

TABLE S4
Adverse events of grade ≥3

| | | | Post- |
|---|---|---|---|
| | | Pre- | Vaccine |
| | All | Vaccination | Initiation |
| | Periods(ITT | (ITT Set, | (Vaccinated |
| | Set, N = 38) | N = 38) | Set, N = 21) |
| Adverse events reported by ≥2% vaccine Initiated | | | |
| patients | | | |
| Hyponatraemia | 2 (5.3) | 2 (5.3) | 1 (4.8) |
| Pneumothorax | 2 (5.3) | 1 (2.6) | 1 (4.8) |
| Atrioventricular block complete | 1 (2.5) | 0 | 1 (4.8) |
| Blood triglycerides increased | 1 (2.6) | 0 | 1 (4.8) |
| Bronchostenosis | 1 (2.6) | 0 | 1 (4.8) |
| Diarrhoea | 1 (2.6) | | 1 (4.8) |
| Dyspnoea | 1 (2.5) | 0 | 1(4.8) |
| Fall | 1 (2.6) | 0 | 1 (4.8) |
| Respiratory failure | 1 (2.6) | 0 | 1(4.8) |
| Adverse events reported by ≥1% ITT patients that were considered | | | |
| related to either NEO-PV-1 and/or any other study drugs | | | |
| Hyponatremia | 1 (2.6) | 0 | 1 (4.8) |
| Anaemia | 3 (7.9) | 3 (7.9) | 0 |
| Neutropenia | 2 (5.3) | 2 (5.3) | 0 |
| Alanine aminotransferase increased | 1 (2.6) | 1 (2.6) | 0 |
| Dehydration | 1 (2.6) | 1 (2.6) | 0 |
| Gastroenteritis | 1 (2.6) | 1 (2.6) | 0 |
| Infection | 1 (2.6) | 1 (2.6) | 0 |
| Nausea | 1 (2.6) | 1 (2.6) | 0 |
| Pneumonia aspiration | 1 (2.5) | 1 (2.6) | 0 |
| Vomiting | 1 (2.6) | 1 (2.6) | 0 |

Numbers and percentages of patients with adverse events of NCI CTCAE grade ≥3 since first dose of study drug

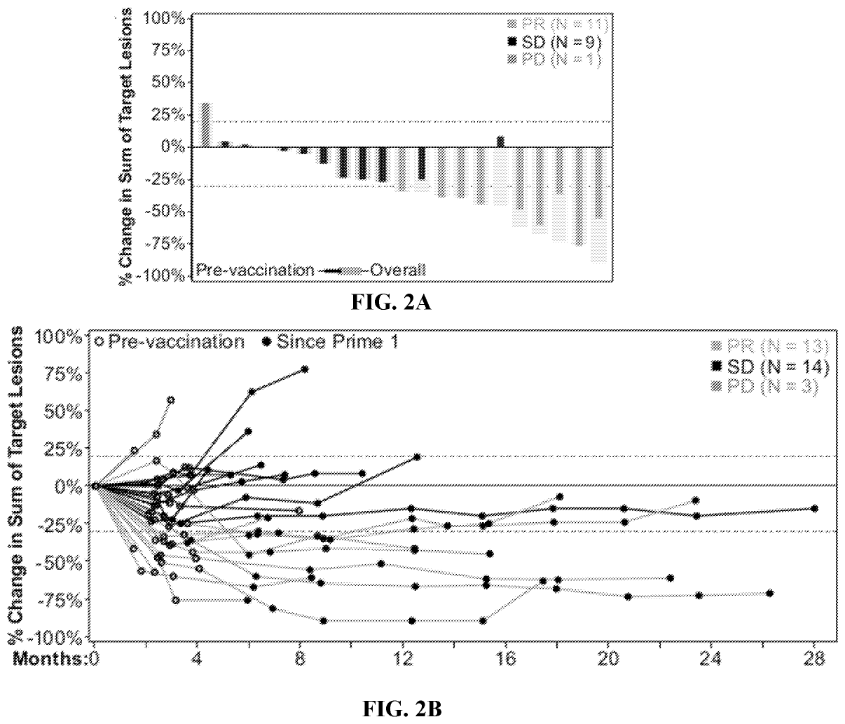

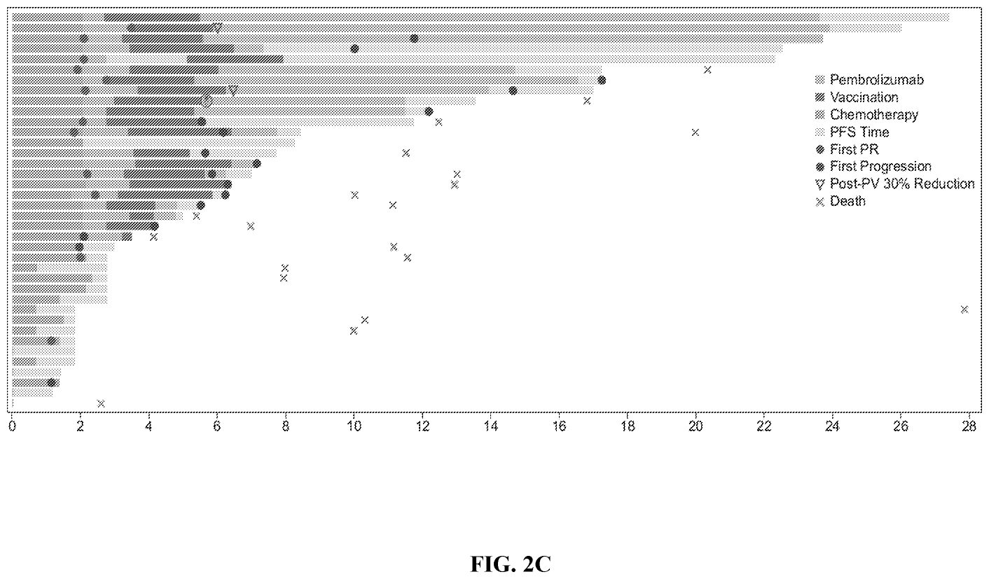

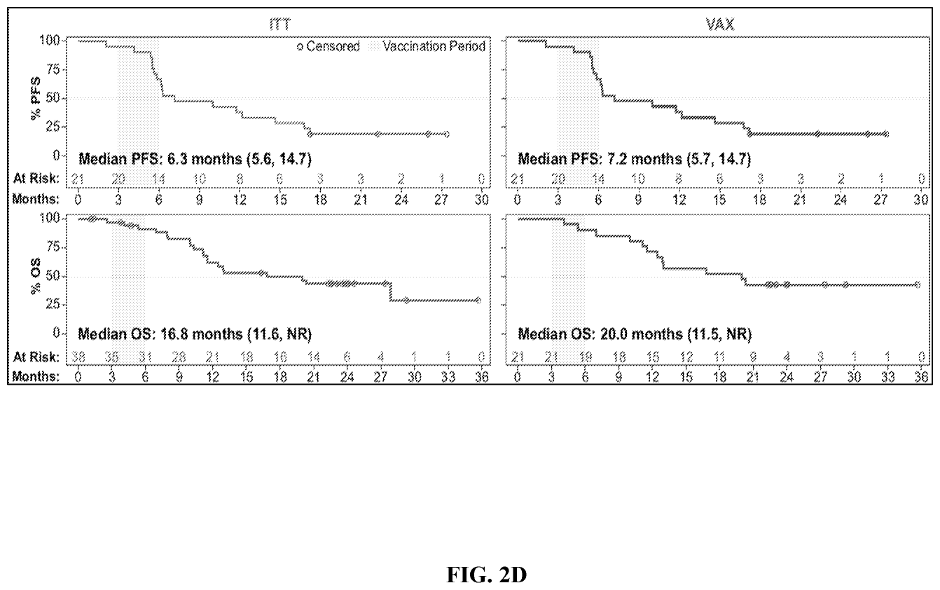

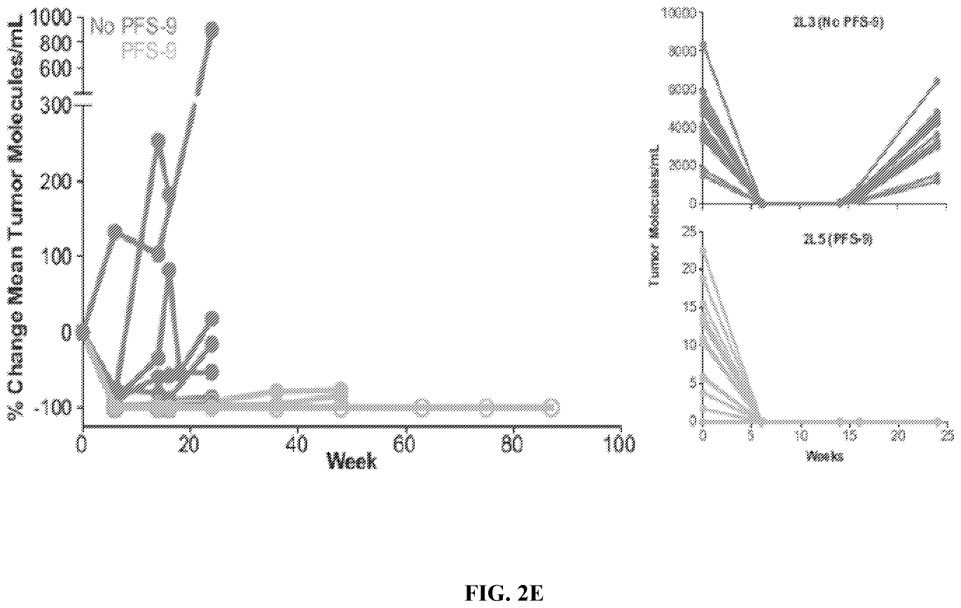

ORR (RECIST v1.1, patients who achieve CR or PR), clinical benefit rate (CBR, patients who achieve CR, PR or SD), PFS and OS were analyzed with a minimum follow-up of 24 months as of the August 2021 data cutoff. FIGS. 2A and 2B summarize the radiographic response profiles (RECIST 1.1) for each patient (change in sum of target lesions) for the VAX set and a subset of the 117 set respectively. Only patients with at least one post-baseline RECIST assessment are shown in FIG. 23. The pair of nested bar plots (FIG. 2A) for the vaccinated patients allows for the comparison of the best response before vaccine initiation (darker shade) and the best response overall (lighter shade). The difference indicates further reduction in tumor size following the initiation of vaccine. Among the 21 patients who received at least one dose of NEO-PV-01, two patients had a further decrease in the sum of target lesion size of more than 30% post vaccination (FIG. 2A). A Swimmer's plot for all patients in the ITT set gives detailed information on treatment schedules for each patient, as well as time of progression, and indicates any case where a patient achieved a post-vaccination reduction of tumor size by at least 30% (FIG. 2C).

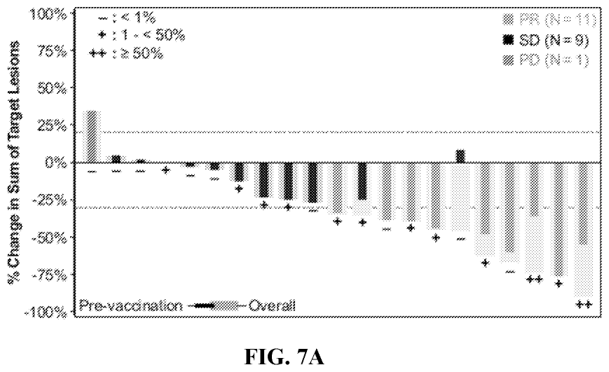

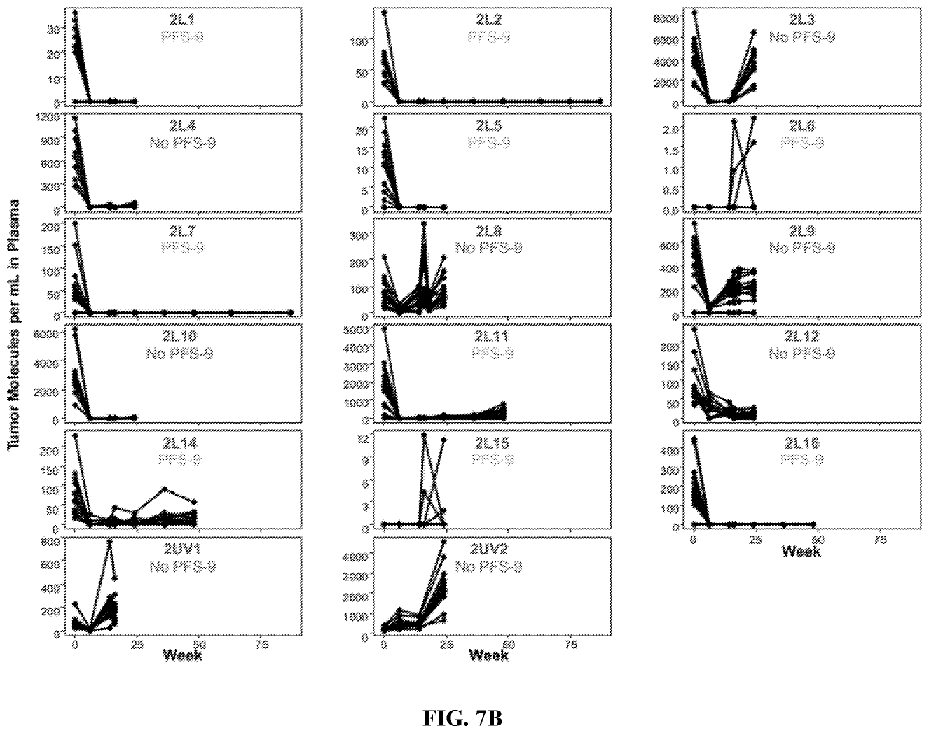

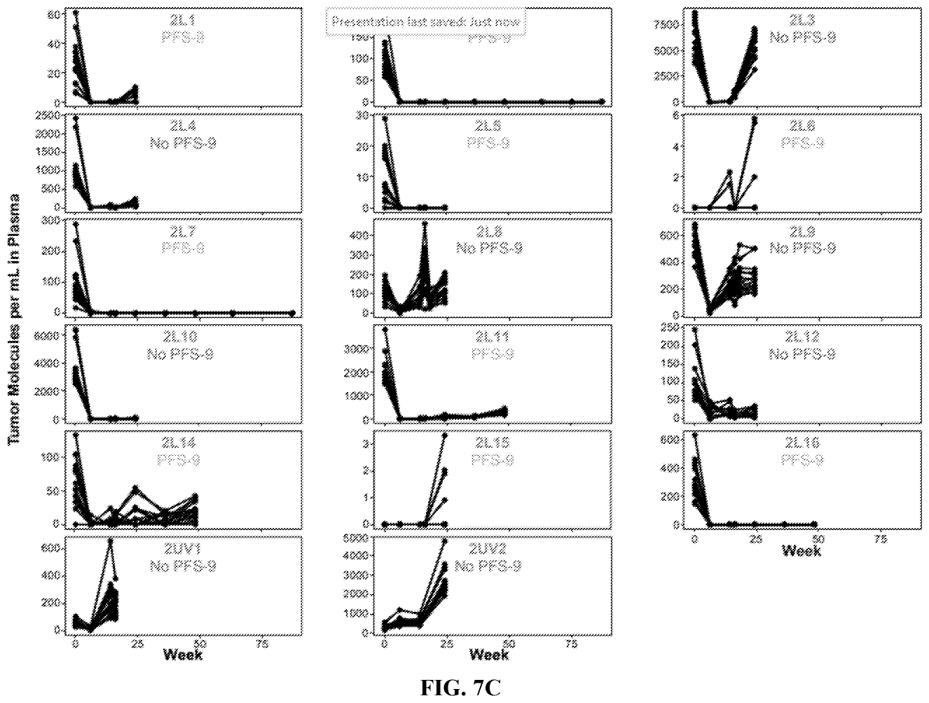

The ORR/CBR (with 95% confidence interval [CI]) for the IT and VAX sets were 34%/71% (19.6-51.4/54.1-84.6) and 69%/100% (41.3-89.0/79.4-100), respectively. Kaplan-Meier estimates for PFS and OS in both the ITT and vaccinated cohorts are shown in FIG. 2D. Median PFS [95% CI] for IT patients was 6.3 months [5.6, 14.7] and for VAX patients was 7.2 months [5.7, 14.7]. Median OS for IT patients was 16.8 months and for VAX patients was 20 months [11.6, NR IT set, 11.5, NR VAX group]. Tumor PD-L1 status for patients in the VAX group is shown in FIG. 7A. Consistent with checkpoint therapy (Hellmann et al., 2018), patients with higher tumor PD-L1 staining had better clinical outcomes. Among the VAX set of 21 patients, 4 out of 5 patients (80%) who progressed during vaccination phase and 5 out of 16 patients (31.25%) who completed vaccination (Full VAX set) had no PDL1 expression on tumor cells at the pre-treatment time point.

Levels of circulating tumor DNA (ctDNA) in the peripheral blood plasma have been shown to be indicative of overall tumor burden and to predict relapse and response in multiple solid tumor indications (Abbosh et al., 2017; Corcoran and Chabner, 2018; Snyder, Morrissey and Hellmann, 2019; Moding et al., 2020). In this study, two personalized and tumor-informed ctDNA assays (Vandekerkhove et al., 2021; Powles et al., 2021) were designed and tested for each patient to measure the levels of ctDNA. 16 of these 32 variants were predicted to be high quality neoepitopes based on our internal bioinformatics algorithms previously described in (Ott et al., 2020) (FIG. 7B). The other set of 16 variants cannot have been included in the vaccination depending on the overlap between the Natera and our variant algorithm selection output but were shown to be tumor-specific (FIG. 7C). For this post-hoc analysis, an exploratory endpoint was used defined by lack of progression at 9 months (PFS-9) post initiation of treatment with pembrolizumab and chemotherapy. A median PFS of 8.8 months was observed previously with pembrolizumab plus chemotherapy in first line metastatic non-squamous non-small cell lung cancer patients (Gandhi et al., 2018). Overall, the mean neoantigen change of all variants tested per patient remained undetectable for patients who achieved PFS-9 (9 patients), while the majority of patients who did not achieve PFS-9 (no PFS-9, 8 patients) showed stable or increasing amounts of neoantigen-specific ctDNA molecules (FIG. 2E, left). FIG. 2E, right provides an example of the data at the individual neoantigen level for a patient who did not achieve PFS-9 (2L3, top), and a patient who did achieve PFS-9 (2L5, bottom). Interestingly, the overall pattern of change in ctDNA levels is consistent across all mutations measured for these two patients, suggesting that no one neoantigen is able to escape under conditions of tumor control, and in the case of progressive disease, all previously detected neoantigens are present during a period of tumor regrowth.

Correlates with clinical response are observed pre-treatment in the tumor microenvironment (TME), including T cell infiltration, MHC Class II expression, and TCR diversity.

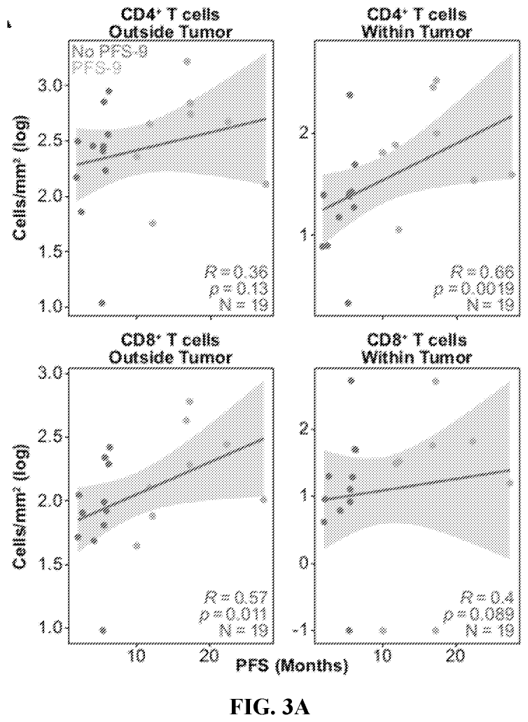

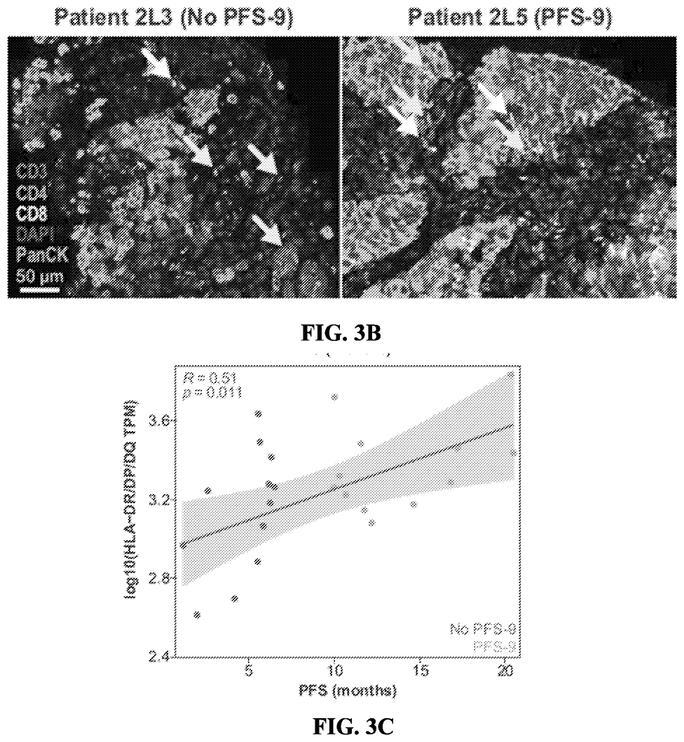

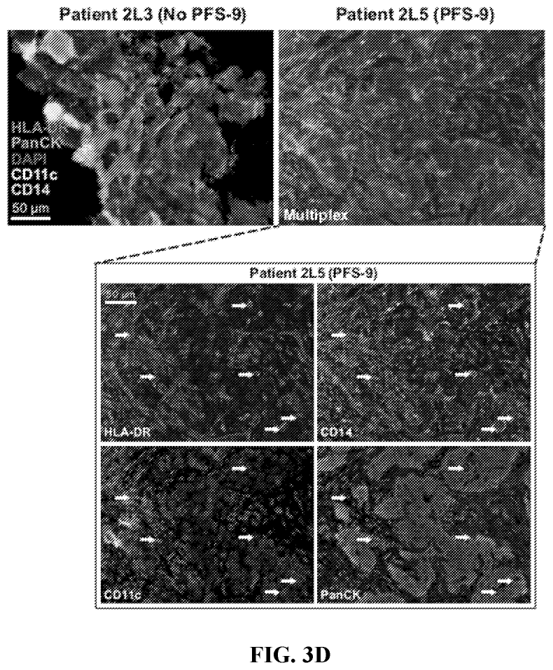

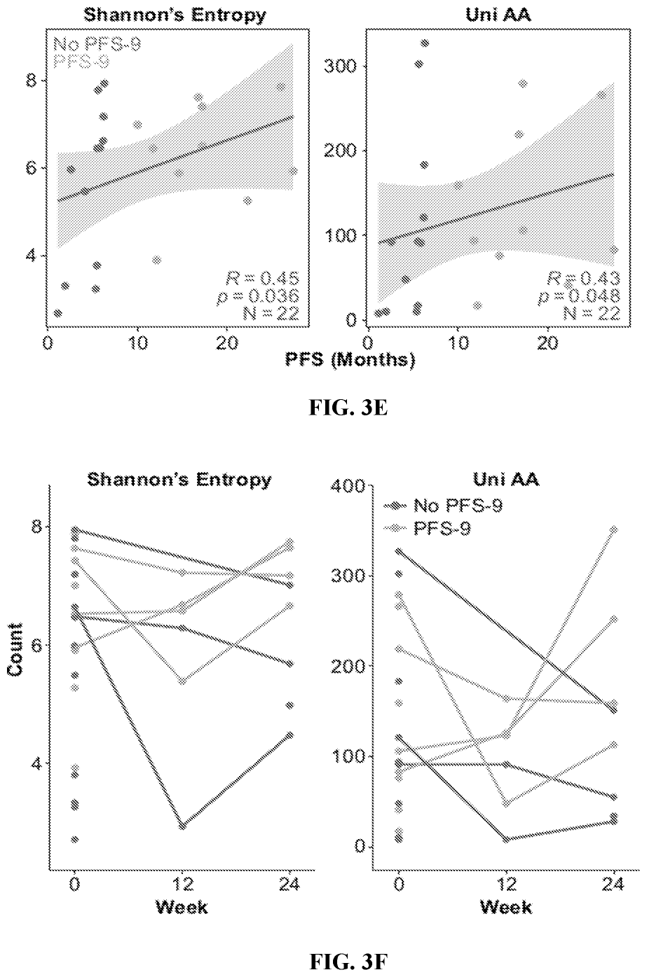

It has been shown previously that the infiltration of T and B cells into the tumor correlates with response to checkpoint therapy (Ganesan et al., 2017; Helmink et al., 2020; Petitprez et al., 2020; Cabrita et al., 2020; Federico et al., 2021). Multiple immune infiltrate readouts were also observed to correlate with clinical responses in our study. First, infiltration of both CD4+ and CD8+ T cells into the tumor microenvironment at the pre-treatment timepoint were observed to correlate with clinical response, suggesting that the immune state of the tumor before the patient undergoes treatment can predict response to the treatment regimen (FIG. 3A). Analysis of the specific localization of CD4+ and CD8+ T cells revealed that infiltration of CD4+ T cells into areas with tumor cells correlates positively with response, whereas the localization of CD8+ T cells outside of the tumor areas correlates positively with response (FIG. 3A). An example of a patient who achieved PFS-9 (2L5) and a patient who did not achieve PFS-9 (2L3) is shown in FIG. 3B. Second, it was observed that HLA Class II gene expression in the tumor biopsies at the pre-treatment timepoint correlates positively with clinical outcome (FIG. 3C). This is further supported by analysis for Class II expression at the protein level by multiplex IHC. Strong MHC Class II staining was observed specifically in cells of the monocyte lineage (CD14, CD11c) in the tumor microenvironment for patient 2L5 who achieved PFS-9 when compared to a patient who did not achieve PFS-9 (2L3) (FIG. 3D). MHC Class II expression by cells of the monocytic lineage in the tumor microenvironment suggest the presence monocyte-derived DCs and macrophages that can provide targets for neoantigen-specific CD4+ T cells, leading to better tumor control for patients with high expression of MHC Class II. Analysis of MHC class II staining in available longitudinal biopsies did not reveal any association of changes in HLA DPDQDR positive cells in the tumor with clinical outcome of PFS-9. The TCR repertoire in the pre-treatment TME was also assessed both by calculating the normalized Shannon's Entropy (Jia et al., 2015; Hanson et al., 2020), as well as overall Complementarity Determining Region (CDR3) unique amino acid sequences (AAs). This analysis revealed a positive correlation between PFS and TCR diversity (FIG. 3E). Applicants were also able to monitor the changes in TCR diversity over time in a smaller subset of post-treatment tumor biopsies. An overall pattern towards increased or stable diversity was observed over the course of the study for patients who achieved PFS-9, while those who did not achieve PFS-9 generally show decreasing TCR diversity over the course of the study (FIG. 3F).

NEO-PV-01 plus chemotherapy and anti-PD-1 induces neoantigen-reactive T cell responses that are neo-epitope specific, persistent, and show cytotoxic potential.

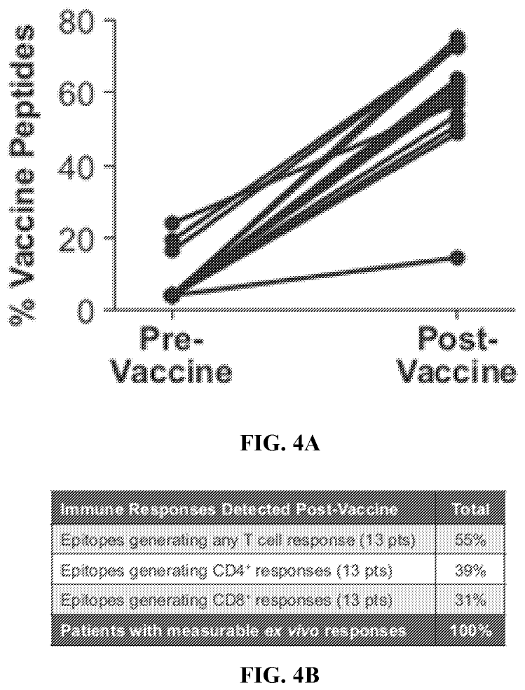

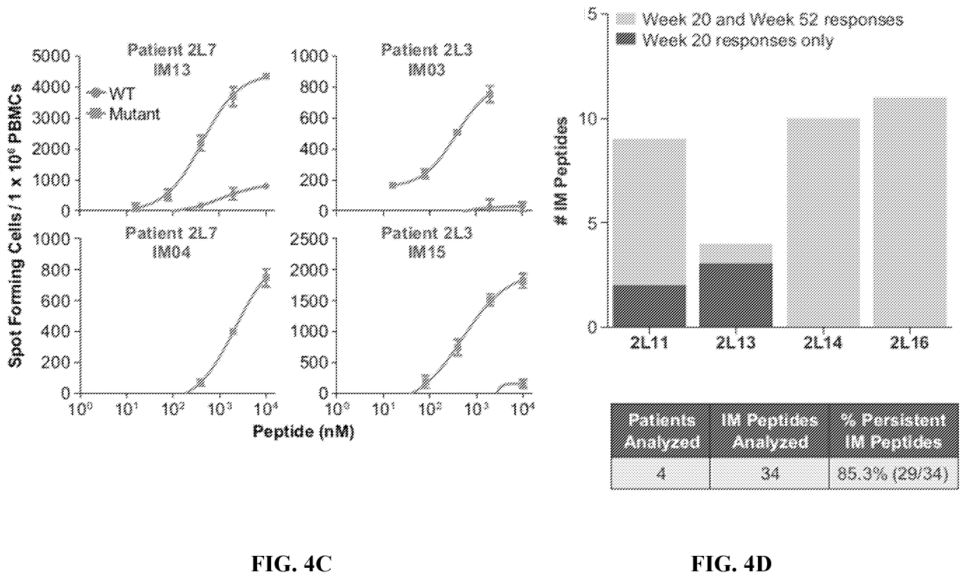

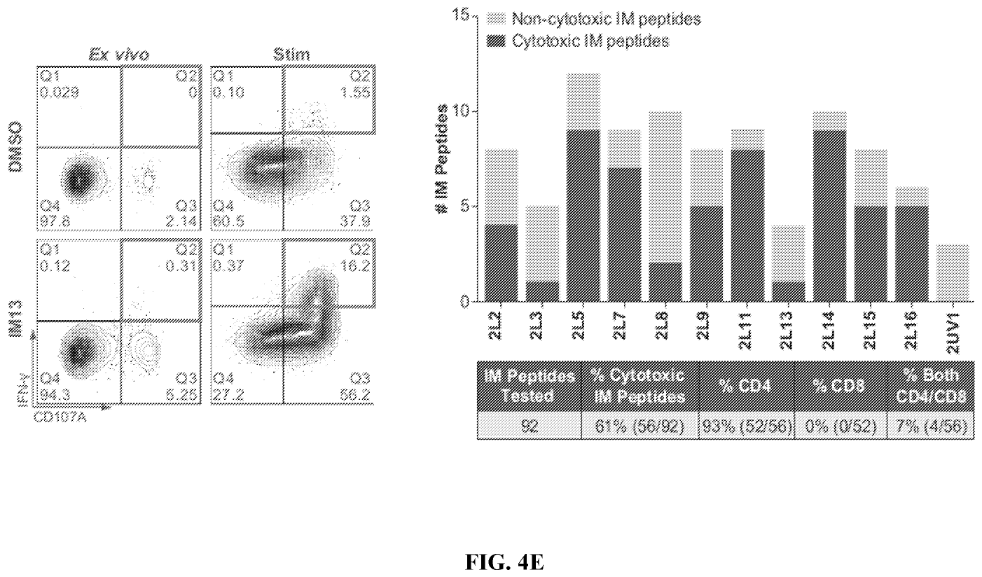

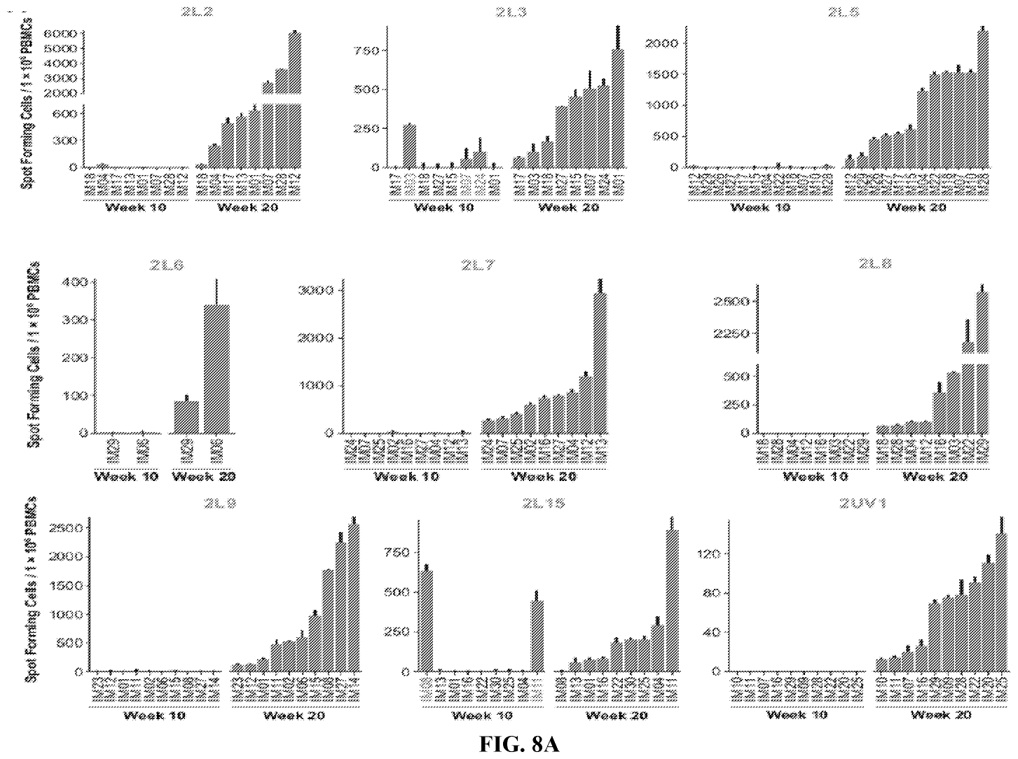

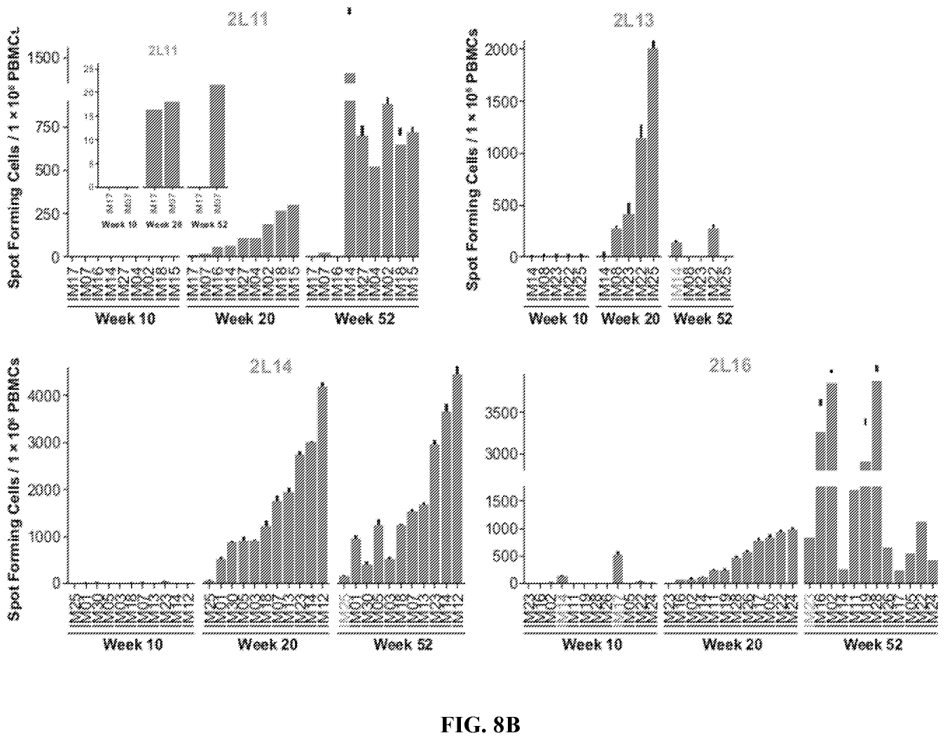

The overall immunogenicity of NEO-PV-01 plus chemotherapy and anti-PD-1 was assessed in patients' peripheral blood by measuring IFNγ secretion in response to recall with the vaccinating peptides. In total, T cell responses were assessed for 204 vaccinating peptides across 13 patients with available samples. Consistent with our previous report (Ott et al., 2020), the percentage of vaccine peptides generating an immune response in this assay was minimal at the pre-vaccine timepoint and increased across all 13 patients assessed at the post-vaccine timepoint (8 weeks after the first dose of vaccine) (FIG. 4A, FIG. 8A). An average of 55% of vaccine epitopes generating any T cell response were observed using this assay (FIG. 43). Of these responses, 49% were detectable ex vivo in an overnight assay with added peptide, and the remaining responses were seen after a 5-day stimulation with added peptide. Overall, 100% of patients elicited at least one measurable response ex vivo (FIG. 43). 94% of the immune responses to the vaccine peptides were seen only in the post vaccination time point.

The percentage of epitopes generating either a CD4+ or CD8+ T cell response for each patient was measured across the 13 patients analyzed, resulting in 39% of epitopes generating a CD4+ T cell responses, while 31% generated CD8+ T cell responses (FIG. 43). The specificity of these immune responses to the neo-epitope as compared to the wild-type epitope was also tested across a range of peptide concentrations. Stronger reactivity to the neo-epitope was observed in 18 of 19 epitopes tested (95%) across 5 patients (FIG. 4C, Table S5). These data are consistent with the specificity of the immune response for neoantigens as previously reported (Ott et al., 2017; Ott et al., 2020; Hu et al., 2021).

TABLE S5
Specificity of NEO-PV-01 induced immune responses to the mutant epitope versus
wild-type epitope. Table discloses SEQ ID NOS 6-43, respectively, in order of columns.

| | Mutant peptide | | Cross reactive to |
|---|---|---|---|
| Peptide ID | sequence | WT peptide sequence | WT |
| 2L2-IM04-ASP0010 | RPRSWLELPLRLAD | RPRSWRELPLRLAD | No |
| 2L2-IM07-ASP0061 | PGTHLAKILSDMRSQ | PGTQLAKILSDMRSQ | No |
| 2L2-IM12-ASP0044 | ERRVANNARELLRVR | ERRVANNARERLRVR | No |
| 2L2-IM13-ASP0041 | LQRFHYSKVNKSDDTAE | LQRFLYSKVNKSDQTAE | No |
| 2L2-IM28-ASP0054 | PAGIRGPYMFQWNDDR | PAGIGGPYMFQWNDDR | No |
| 2L3-IM01-ASP0033 | IKTQWITSFYKSSLKD | IKTVWITSFYKSSLKD | No |
| 2L3-IM03-ASP0031 | AKERARVADSNRQLAL | AKERARVADSQRQLAL | No |
| 2L3-IM15-ASP0065 | RFLNSFASMLRFGPI | RFLNSFASMHRFGPI | No |
| 2L3-IM24-ASP0010 | KRLARVLSVDSGTDV | KRHARVLSVDSGTDV | No |
| 2L5-IM04-ASP0020 | ALDSFLRENDVQFTV | ALDSFLRLNDGQFTV | No |
| 2L5-IM10-ASP0050 | TRNVDVQLLDTKRQL | TRNLDVQLLDTKRQL | No |
| 2L5-IM15-ASP0009 | ETNRARILAPLLIVDK | ETNRARILAPLLILDK | No |
| 2L5-IM22-ASP0018 | SRHLALSWPTFGIIVK | SRHLALSGPTFGIIVK | No |
| 2L5-IM29-ASP0016 | KRPCPWSEPSVGTTL | KRPGPWSEPSVGTTL | Yes |
| 2L7-IM02-ASP0035 | TVPIYEVYALPHAILR | TVPIYEGYALPHAILR | No |
| 2L7-IM04-ASP0020 | TVEAEAAHVTVTRHY | TVEAEAAHGTVTRHY | No |
| 2L7-IM13-ASP0018 | QQSALRVMQWNCAAYLK | QQSALRVMQRNCAAYLK | No |
| 2L7-IM16-ASP0065 | RVFNNVLLQQTQRLD | RVFNNVLLQQTQHLD | No |
| 2L7-IM19-ASP0053 | PVPEGAGLKGHLHKL | PVPEGAGLKGHLPKL | No |

Persistence of the vaccine-induced immune responses was also assessed for a subset of patients at 52 weeks after initiating the study (29 weeks after the final dose of vaccine). Of the 34 epitopes tested at week 52, 85.3% still had a response present at this late timepoint (FIG. 4D, FIG. 8B). Responses were also observed at week 52 that were not present at the week 20 post-vaccination timepoint for patients 2L13 (IM14), 2L14 (IM25) and 2L16 (IM23), indicating the potential for delayed generation of vaccine-induced responses (FIG. 8B, green). A pre-existing immune response was also observed for p53 R213L (IM14) neoantigen in patient 2L16 that was also observed at the week 52 timepoint. (FIG. 8B). These data suggest that overall, the immune responses generated in this study persisted for up to at least one year.

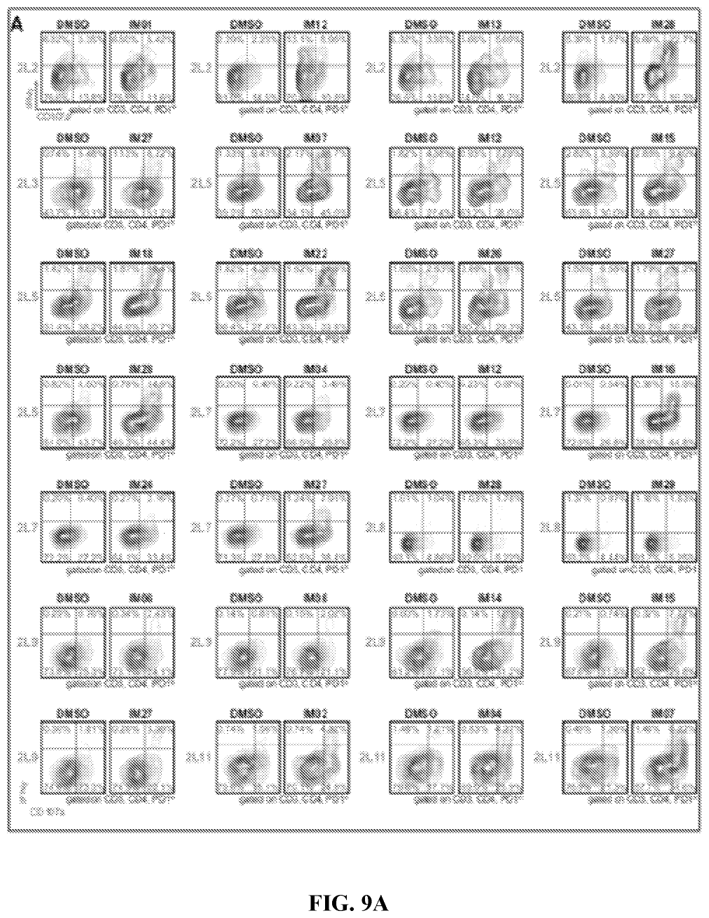

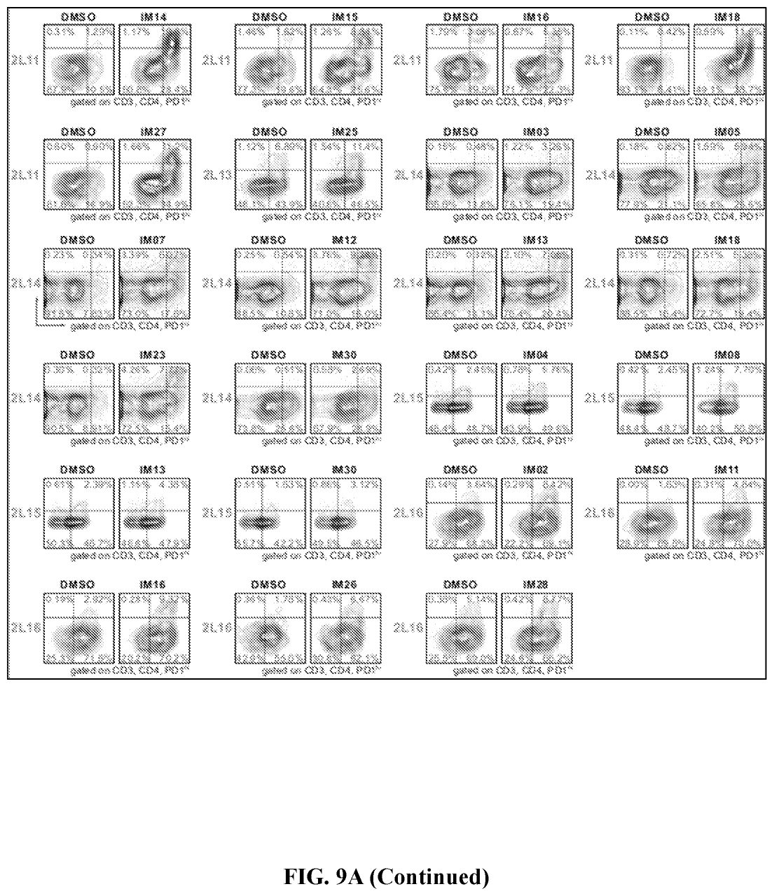

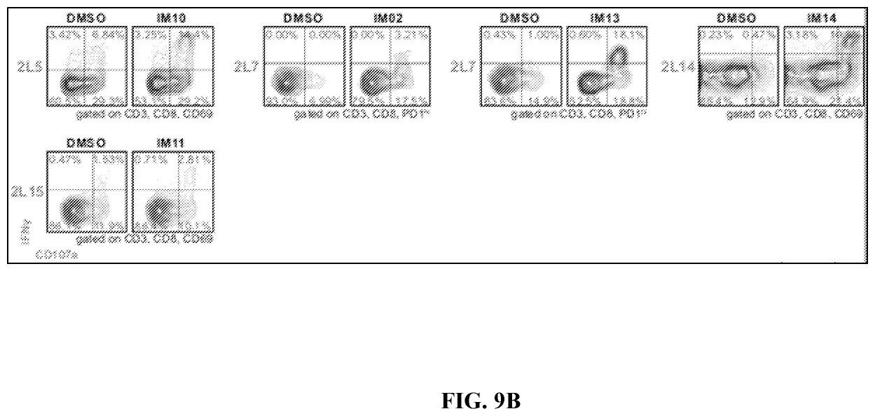

The cytotoxic potential of the T cell responses generated against the vaccinating peptides was assessed by measuring the level of co-expression of IFNγ and CD107a (a marker of degranulation and cytotoxic potential (Betts et al., 2003)) expression on CD4+ and CD8+ T cells, when assayed in the presence of neoantigen peptide (FIG. 4E, flow panel insert). In total, 92 vaccinating peptides were tested across 12 patients. Co-expression of IFNγ and CD107a was observed for 56 of the 92 (61%) peptides tested, with 11 out of 12 patients showing at least one cytotoxic response (92%) (FIG. 4E, FIGS. 9A and 9B). Interestingly, when each cytotoxic response was characterized as either a CD4+ or CD8+ T cell response based on the subset of T cells which showed increased co-expression when compared to the DMSO-treated control cells, 93% of responses were characterized solely as CD4+ responses, and the remaining 7% showed a response in both the CD4+ and CD8+ compartment (FIG. 4E, table).

NEO-PV-01 plus chemotherapy and anti-PD-1 induces epitope spread responses in the majority of patients analyzed, with mutant KRAS responses observed in multiple patients.

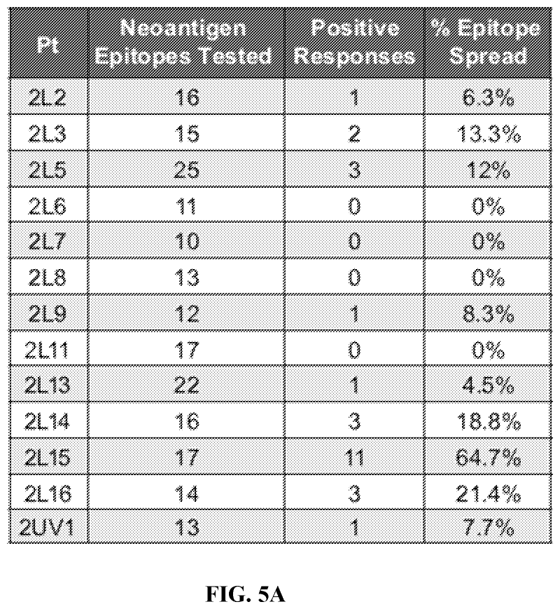

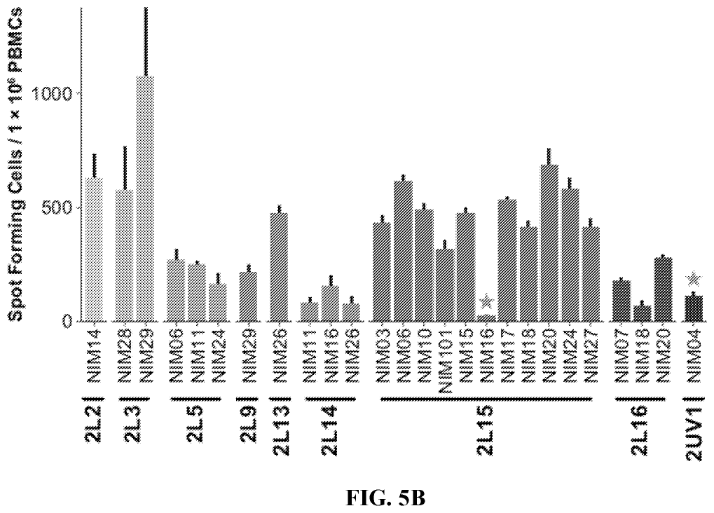

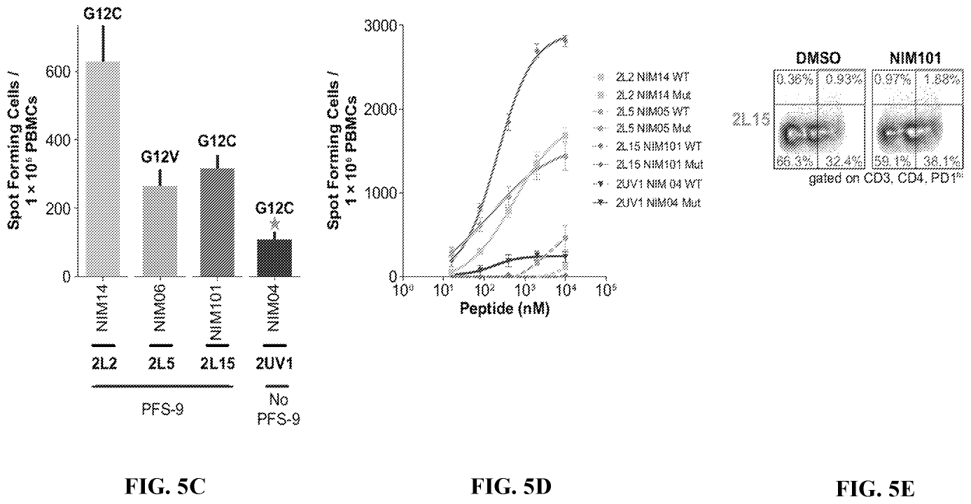

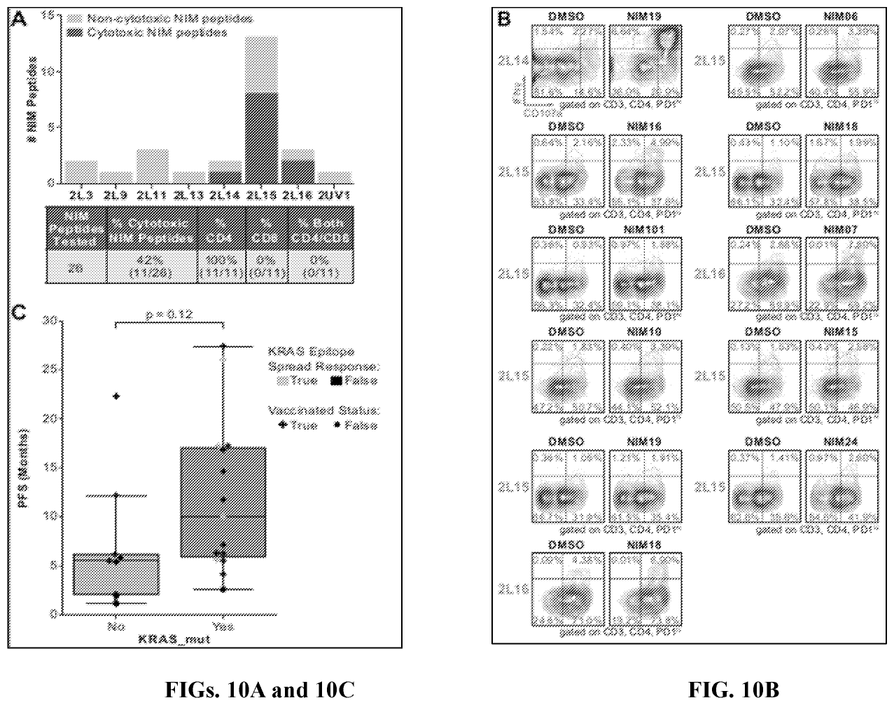

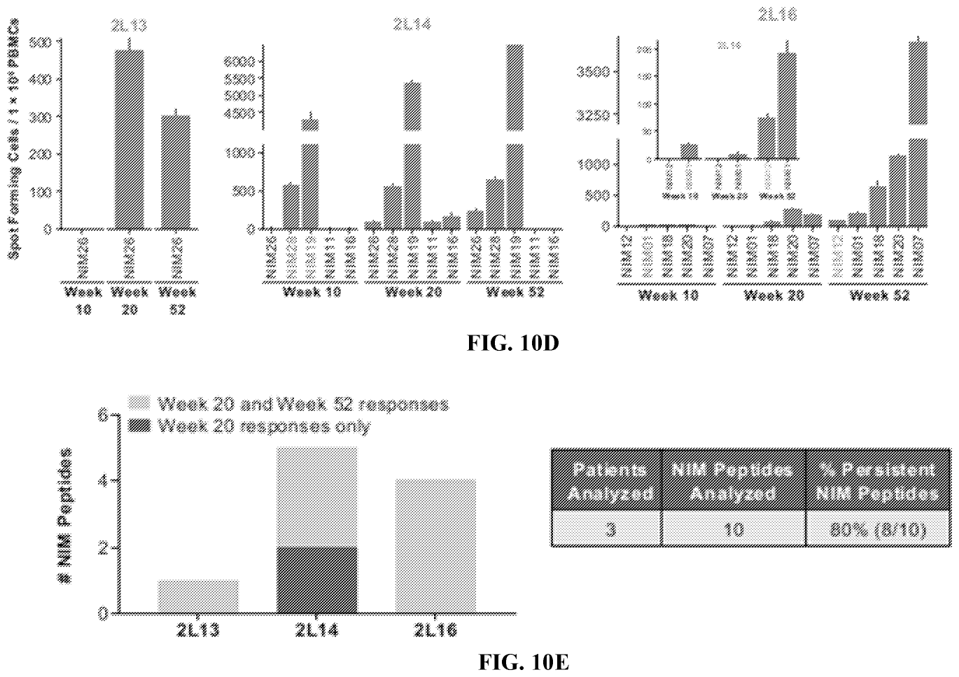

Given the cytotoxic phenotype of many of the T cell responses against the neoantigen vaccine epitopes, it was speculated that tumor cell killing by these T cells could reveal additional epitopes, boosting the overall immune response to the patients' tumor. This concept, known as epitope spread, has been reported in a number of studies assessing T cell responses to vaccines (Corbiere et al., 2011; Kreiter et al., 2015; Ott et al., 2020; Hu et al., 2021). To quantitate epitope spread in this study, additional circulating T cell responses were analyzed to highly ranked neoepitopes that were not included in the vaccine (referred to as “non-immunizing peptides”, or “NIMs”). Immune responses that were detected only at the post-vaccine timepoint (week 20) were considered as epitope spread responses. Across 13 patients analyzed, 9 patients (69%) demonstrated epitope spread (FIGS. 5A and 5B) Of note, was the epitope spread responses that were seen to the driver mutations. Of 19 patients that harbored KRAS mutations in the study, immune responses were measured for seven of them. Three patients had an epitope spread response to G12C and one patient had a response to a G12V epitope (FIG. 5C and Table Si). Of the 4 patients that had epitope spread to a KRAS mutation, 3 of them achieved PFS-9 on the study. The three patients where epitope spread to mutant KRAS was not observed harbored the mutations G12V, G12C and G12A respectively. Stronger reactivity to the neo-epitope when compared to wild type was demonstrated in all 4 patients where an epitope spread response to a KRAS mutant epitope was observed (FIG. 5D). Additionally the cytotoxic potential of these epitope spread responses were assessed by measuring co-expression of IFNγ and CD107a as in FIG. 4E. Of the 26 NIMs tested, 11 (42%) were cytotoxic, with 3 of 8 patients showing at least one cytotoxic response (38%). 100% of the cytotoxic responses observed were CD4+ responses, further supporting the importance of anti-tumor CD4+ responses in this study (FIGS. 10A and 10B). Interestingly, the KRAS G12C specific CD4+ response (NIM101) from patient 2L15 showed cytotoxic potential in this assay (FIG. 5E). The presence of KRAS mutations in our study was positively associated with improved PFS (FIG. 10C) suggesting the immunogenicity to KRAS mutations can in part be driving better clinical outcomes with our treatment regimen. Persistence of these epitope spread responses was also assessed at the week 52 timepoint for three patients with available sample (FIGS. 10D-10E). Of the 10 epitopes tested, 80% were also detected at this later timepoint, consistent with the observation made to the vaccinating epitopes in FIG. 4D. For patient 2L16, an additional epitope spread response (NIM12) was observed at week 52 that was not detectable at week 20 suggesting epitope spread to additional antigens can continue beyond the vaccination time period and could be an effect of continued pembrolizumab treatment beyond the vaccination phase (FIG. 10D, green).

Neoantigen-specific CD4+ T cell responses generated post-vaccination have an activated effector phenotype.

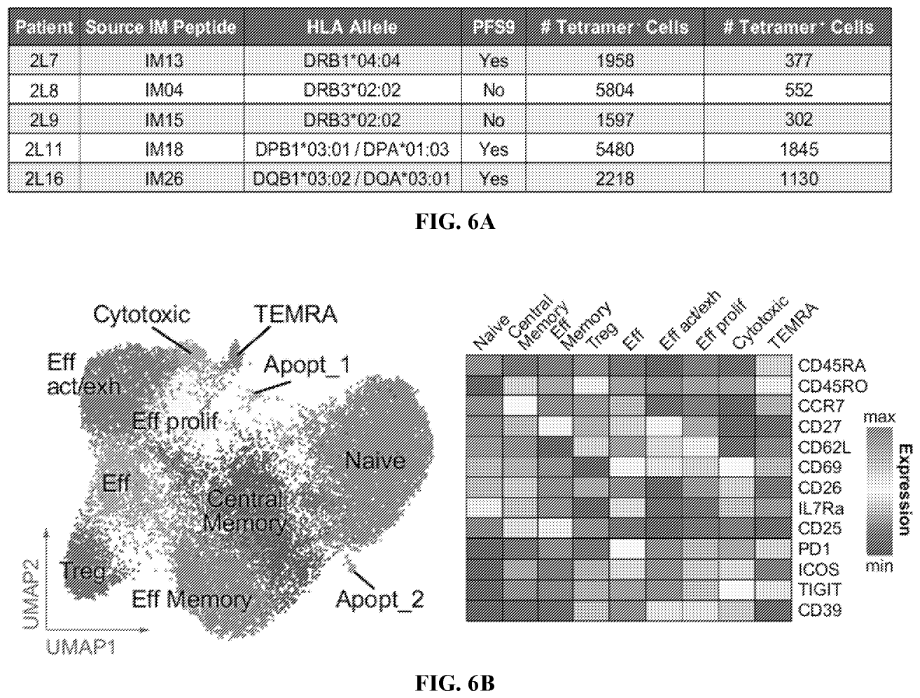

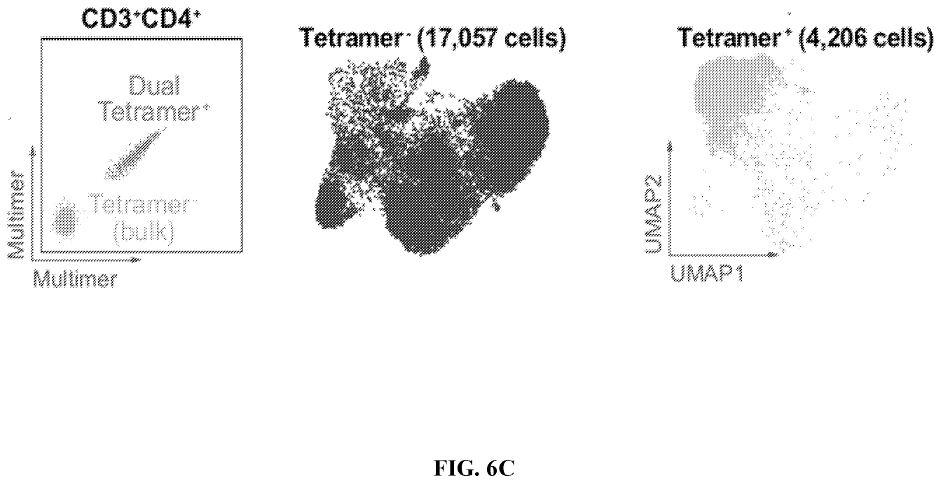

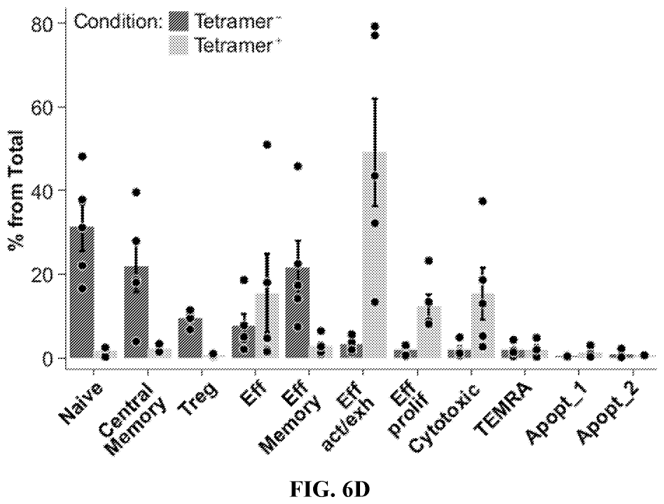

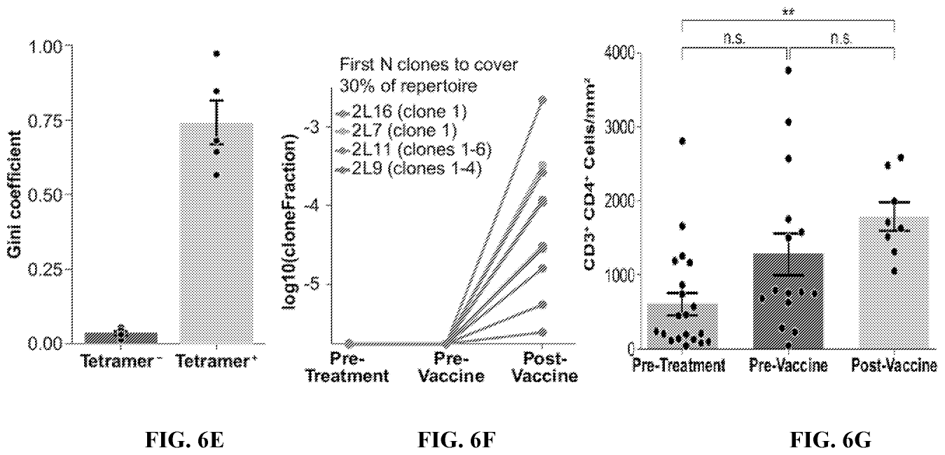

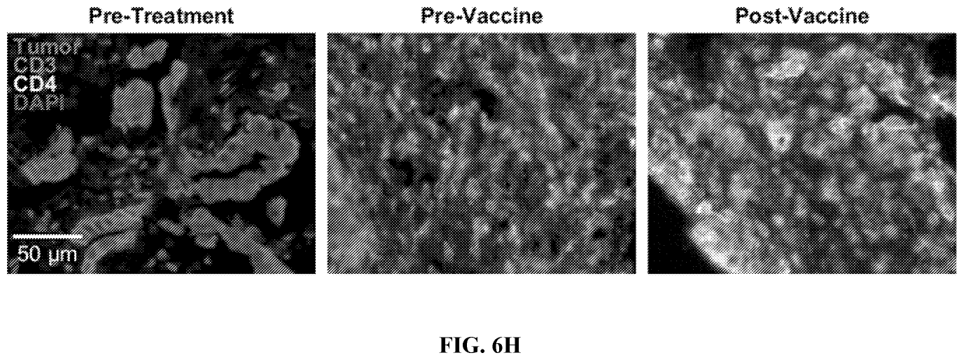

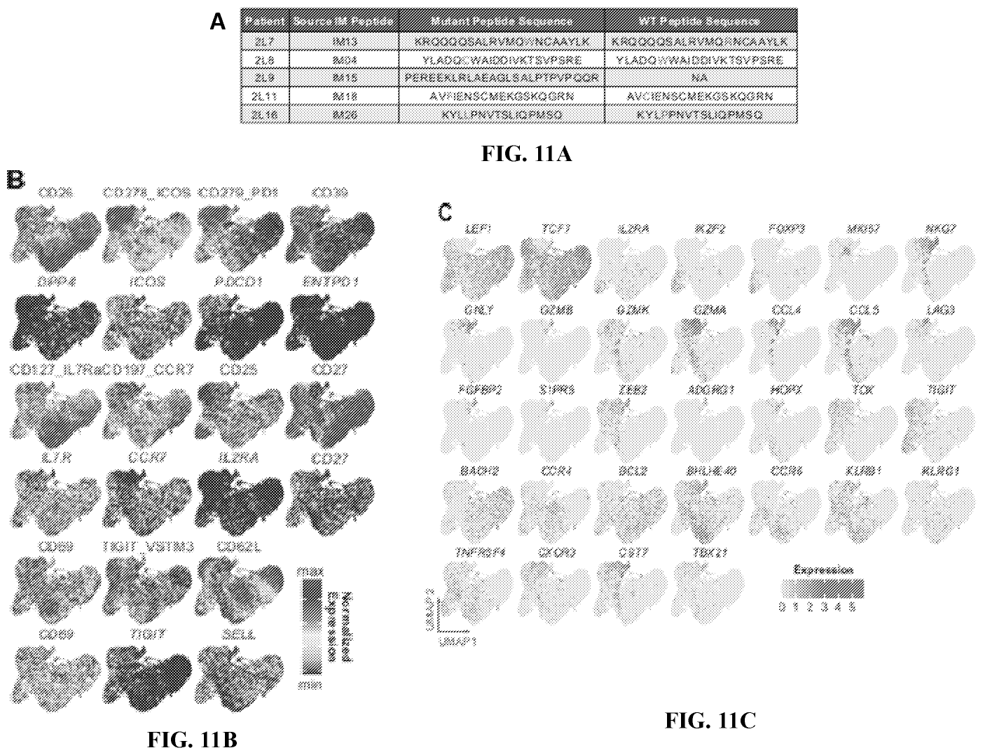

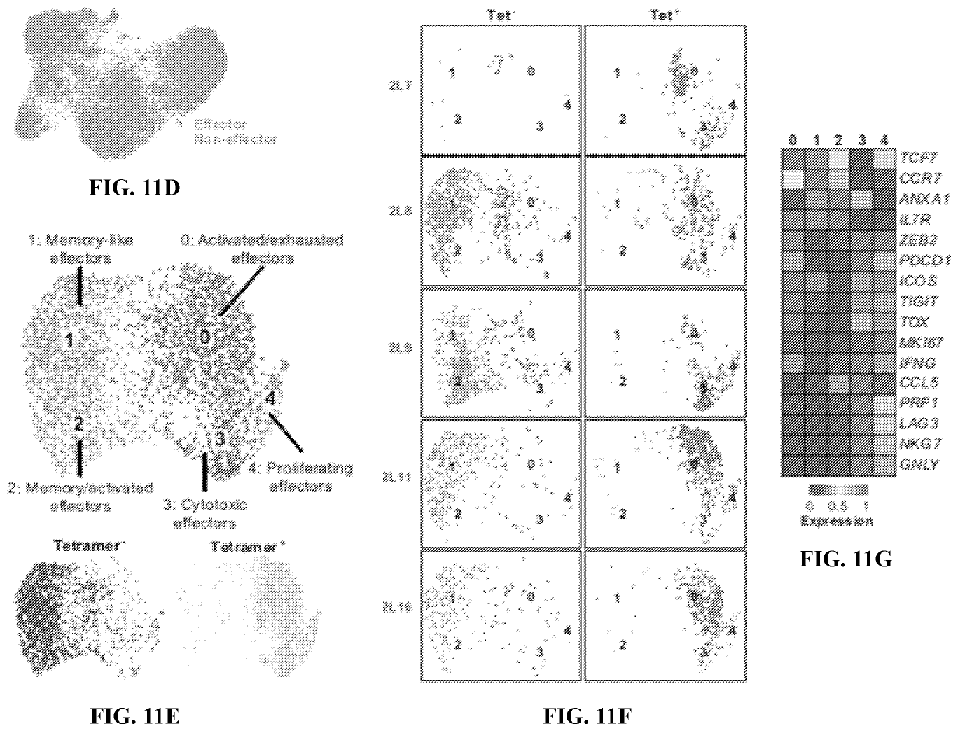

To further elucidate the role of vaccine-induced CD4+ T cells induced in the patients, a detailed transcriptomic and clonotype atlas of these cells was generated by utilizing a combination of single cell sequencing techniques, including TCR-sequencing and Cellular Indexing of Transcriptomes and Epitopes by Sequencing (CITEseq). Using these methodologies, it was possible to overlay detailed single cell-level TCRseq data with protein and gene expression analysis comparing neoantigen-specific CD4+ T cells (tetramer+) with bulk (tetramer−) CD4+ T cells for five patients from peripheral blood at the post-vaccination timepoint (FIG. 6A and FIG. 11A). The addition of protein expression analysis using CITEseq to standard gene expression strengthened the clustering analysis (FIGS. 11B and 11C). Three of these patients achieved PFS-9 on the study and two did not. CD4+ T cell responses representative of all responses in a patient were chosen based on availability of Class II tetramers and the magnitude of the response yielding enough cells for detailed analysis. To perform these analyses, neoantigen-specific CD4+ T cells were sorted using MHC Class II multimers as shown in FIG. 6A, right. Unsupervised clustering of all CD4+ T cells across the five patients revealed commonly observed T cell subsets determined based on the expression profiles, such as naïve CD4+ T cells, central memory, Treg, and effector CD4+ T cells (FIG. 63). Additional analysis demonstrated that the tetramer+ cells for each of the 5 patients analyzed cluster separately from the tetramer− CD4+ T cell population, indicating a difference in transcriptomic and protein profile (FIG. 6C). Indeed, tetramer+ CD4+ T cells fall into the “Effector Activation/Exhaustion”, “Effector Proliferation”, and “Cytotoxic” clusters at a greater frequency than the tetramer− CD4+ T cells, while tetramer− cells fall into clusters of “Naive”, “Central Memory” and “Effector Memory” cells. (FIG. 6D). Differential gene expression analysis further underscored the difference in cluster distribution and phenotypes between tetramer+ and tetramer− populations. ZEB2, PDCD1, TOX, TIGT, CXCR3, ITGB1, GZMA and ICOS were all within the top 100 genes significantly upregulated in the tetramer+ (neoantigen-specific) CD4+ T cell populations (Tables S6 and S7). Additionally, when the clonality of the TCR repertoire was compared across the tetramer+ and tetramer− populations as measured by the Gini coefficient (Kirsch, Vignali and Robins, 2015; Poran et al., 2020), increased clonality of the neoantigen-specific tetramer+ populations was observed for each of the 5 patients when compared to the tetramer− CD4+ T cell population (FIG. 6E).

Table 6. Analysis of Differentially Expressed Cell Surface Markers Between Tetramer+ Vs. Tetramer− CD4+ T Cells.

TABLE 6(A)
Differential expression analysis was performed using cell
surface marker expression of CITE antibodies between all
tetramer+ and tetramer- CD4+ T cell populations
from five patient samples analyzed with single-cell RNA-Seq.

| | | | | Adjusted |
|---|---|---|---|---|
| Gene | Avg Log2(Foldchange) | Pct1 | Pct2 | p value |
| cite-CD278-ICOS | 0.639857147 | 1 | 1 | 0 |
| cite-CD279-PD1 | 1.155932032 | 0.981 | 0.587 | 0 |
| cite-CD39 | 0.613011096 | 0.89 | 0.556 | 0 |
| cite-CD45RO | 0.457437842 | 0.991 | 0.828 | 0 |
| cite-TIGIT-VSTM3 | 0.638189182 | 0.938 | 0.67 | 0 |
| cite-CD27 | −0.193119857 | 0.979 | 0.995 | 2.66E−13 |
| cite-CD127-IL7Ra | −0.599335045 | 0.882 | 0.988 | 0 |
| cite-CD197-CCR7 | −0.445104549 | 0.642 | 0.942 | 0 |
| cite-CD25 | −0.679487879 | 0.367 | 0.87 | 0 |
| cite-CD26 | −0.913393393 | 0.823 | 0.991 | 0 |
| cite-CD45RA | −1.130035218 | 0.631 | 0.967 | 0 |

TABLE 6 (B)
Differential expression analysis using cell surface marker expression
of CITE antibodies between effector tetramer+ and effector tetramer-
CD4+ T cell populations. Only genes with log2FC > 0.15 and
genes expressed in more than 15% of cells were considered for comparison.

| | Avg | | | Adjusted p | |
|---|---|---|---|---|---|
| Gene | Log2(Foldchange) | Pct1 | Pct2 | value | Specificity |
| cite-CD279-PD1 | 0.847150916 | 0.996 | 0.843 | 0.00E+00 | tet+ specific |
| cite-CD39 | 0.63407726 | 0.899 | 0.639 | 1.36E−168 | tet+ specific |
| cite-TIGIT- | 0.461433468 | 0.926 | 0.775 | 3.38E−142 | tet+ specific |
| VSTM3 | | | | | |
| cite-CD45RA | −0.292580851 | 0.594 | 0.943 | 6.75E−206 | tet− specific |
| cite-CD26 | −0.916368148 | 0.832 | 0.986 | 0.00E+00 | tet− specific |
| cite-CD127- | −0.707158443 | 0.882 | 0.992 | 0.00E+00 | tet− specific |
| IL7Ra | | | | | |
| cite-CD25 | −0.723317524 | 0.397 | 0.906 | 0.00E+00 | tet− specific |

### Table 7A-C: Differential Gene Expression of Tetramer+ and Tetramer− CD4+ T Cells.

Table 7(A) Differential expression analysis was performed using gene expression data of tetramer+ and tetramer− CD4+ T cell populations from five patient samples analyzed with single-cell RNA-seq. Only genes with log 2FC >0.15 and genes expressed in more than 15% of cells were considered for comparison. Top 50 most significantly differentially expressed genes are shown here. Differential expression analysis was also performed using gene expression data of effector tetramer+ and effector tetramer− CD4+ T cell populations, with the top 20 genes upregulated in the tetramer+ population shown in Table 7(B), and top 20 genes upregulated in the tetramer− population shown in Table 7(C).

TABLE 7(A)

| | | | | Adjusted p |
|---|---|---|---|---|
| Gene | avg_log2FC | pct. 1 | pct. 2 | value |
| DDI2 | 1.033668364 | 0.348 | 0.089 | 0.00E+00 |
| MCOLN2 | 0.895208953 | 0.276 | 0.065 | 0.00E+00 |
| HNRNPLL | 0.9786899 | 0.575 | 0.287 | 0.00E+00 |
| LIMS1 | 0.935444825 | 0.451 | 0.186 | 0.00E+00 |
| ZEB2 | 1.761866427 | 0.495 | 0.055 | 0.00E+00 |
| ITGA4 | 1.065584008 | 0.64 | 0.355 | 0.00E+00 |
| ARPC2 | 0.782512321 | 0.807 | 0.725 | 0.00E+00 |
| PDCD1 | 1.557888709 | 0.486 | 0.058 | 0.00E+00 |
| ARAP2 | 1.19188711 | 0.553 | 0.246 | 0.00E+00 |
| ENC1 | 0.838474748 | 0.238 | 0.054 | 0.00E+00 |
| CD109 | 0.823372313 | 0.208 | 0.021 | 0.00E+00 |
| DUSP4 | 1.226898478 | 0.558 | 0.2 | 0.00E+00 |
| TOX | 1.129896147 | 0.401 | 0.079 | 0.00E+00 |
| TP53INP1 | 1.12645482 | 0.34 | 0.097 | 0.00E+00 |
| RNF19A | 1.650723856 | 0.798 | 0.468 | 0.00E+00 |
| FAM160B1 | 1.195834606 | 0.453 | 0.144 | 0.00E+00 |
| GRAMD1B | 1.042632361 | 0.286 | 0.044 | 0.00E+00 |
| MAF | 1.550491229 | 0.53 | 0.137 | 0.00E+00 |
| RNF213 | 1.308500383 | 0.804 | 0.586 | 0.00E+00 |
| CD99 | 0.952381585 | 0.719 | 0.549 | 0.00E+00 |
| MT-CO1 | 1.632077656 | 0.993 | 0.994 | 0.00E+00 |
| MT-CO2 | 1.111277338 | 0.983 | 0.99 | 0.00E+00 |
| MT-CO3 | 1.324846488 | 0.982 | 0.988 | 0.00E+00 |
| SLC7A5 | 1.101509777 | 0.633 | 0.384 | 0.00E+00 |
| NEAT1 | 1.444977661 | 0.847 | 0.698 | 0.00E+00 |
| AKAP13 | 0.929633496 | 0.794 | 0.636 | 0.00E+00 |
| MYH9 | 0.879127537 | 0.834 | 0.726 | 0.00E+00 |
| TIGIT | 0.730180549 | 0.311 | 0.097 | 0.00E+00 |
| MXD4 | 0.8606741 | 0.435 | 0.193 | 0.00E+00 |
| COTL1 | 1.058450828 | 0.68 | 0.553 | 0.00E+00 |
| LDLRAD4 | 1.087275066 | 0.471 | 0.215 | 0.00E+00 |
| CEMIP2 | 1.12331742 | 0.617 | 0.376 | 0.00E+00 |
| YARS | 0.863263982 | 0.383 | 0.152 | 0.00E+00 |
| MIR4435- | 0.619793516 | 0.237 | 0.06 | 0.00E+00 |
| 2HG | | | | |
| EML4 | 0.847380569 | 0.845 | 0.725 | 0.00E+00 |
| ADAM19 | 1.067864508 | 0.304 | 0.103 | 0.00E+00 |
| LCP1 | 0.903489196 | 0.707 | 0.57 | 0.00E+00 |
| MYO1F | 0.79073229 | 0.323 | 0.119 | 0.00E+00 |
| MIAT | 1.130761815 | 0.302 | 0.107 | 0.00E+00 |
| NFATC2 | 0.923641964 | 0.585 | 0.355 | 0.00E+00 |
| YWHAB | 0.757322558 | 0.716 | 0.601 | 1.45E−237 |
| CXCR3 | 0.722203438 | 0.337 | 0.129 | 1.80E−234 |
| CYTOR | 0.792991825 | 0.367 | 0.149 | 1.26E−233 |
| SMAD3 | 0.873472909 | 0.376 | 0.161 | 2.97E−231 |
| GK | 0.887779929 | 0.282 | 0.096 | 7.36E−228 |
| PRKX | 1.037583101 | 0.56 | 0.345 | 8.89E−224 |
| HSPA1A | 1.887244741 | 0.421 | 0.213 | 1.05E−223 |
| TNFRSF25 | 0.906766536 | 0.46 | 0.236 | 2.06E−223 |
| NAA50 | 0.828885404 | 0.463 | 0.241 | 8.14E−221 |

TABLE 7(B)

| Gene | avg_log2FC | pct. 1 | pct. 2 | Adjusted p value |
|---|---|---|---|---|
| ZEB2 | 1.5506901 | 0.509 | 0.114 | 1.79E−155 |
| PDCD1 | 1.23E+00 | 0.478 | 0.136 | 1.50E−124 |
| RNF19A | 1.21E+00 | 0.769 | 0.551 | 1.04E−92 |
| TOX | 9.16E−01 | 0.384 | 0.109 | 1.09E−85 |
| MAF | 1.1725504 | 0.522 | 0.247 | 1.37E−80 |
| RNF213 | 1.1674754 | 0.799 | 0.668 | 9.84E−79 |
| DDI2 | 0.9625013 | 0.325 | 0.095 | 4.62E−68 |
| CTLA4 | 0.8215844 | 0.361 | 0.138 | 1.93E−56 |
| GRAMD1B | 0.9112499 | 0.274 | 0.083 | 7.09E−54 |
| IRF4 | 0.869015 | 0.293 | 0.094 | 1.23E−52 |
| MIR4435-2HG | 0.6348039 | 0.289 | 0.094 | 1.08E−50 |
| DUSP4 | 0.7782985 | 0.575 | 0.353 | 4.44E−48 |
| YARS | 0.8231458 | 0.373 | 0.169 | 9.45E−48 |
| BCL2L11 | 0.8985211 | 0.326 | 0.132 | 1.32E−47 |
| LIMS1 | 0.8102664 | 0.458 | 0.257 | 2.74E−47 |
| FAM160B1 | 0.8898139 | 0.379 | 0.182 | 6.63E−45 |
| MXD4 | 0.7901403 | 0.428 | 0.234 | 7.29E−44 |
| ICOS | 0.8310407 | 0.65 | 0.506 | 2.43E−43 |
| TP53INP1 | 0.9382226 | 0.316 | 0.132 | 2.67E−43 |
| CEMIP2 | 0.9154886 | 0.553 | 0.364 | 3.79E−42 |

TABLE 7(C)

| Gene | avg_log2FC | pct. 1 | pct. 2 | Adjusted p value |
|---|---|---|---|---|
| TSC22D3 | −1.2204529 | 0.777 | 0.953 | 3.07E−175 |
| LMNA | −1.776522 | 0.497 | 0.82 | 5.61E−168 |
| FOSB | −1.2628637 | 0.44 | 0.803 | 3.72E−139 |
| IL7R | −0.9888457 | 0.589 | 0.888 | 1.89E−133 |
| FTL | −0.9181035 | 0.601 | 0.859 | 1.64E−116 |
| FAU | −0.7291478 | 0.759 | 0.904 | 7.83E−116 |
| LTB | −0.9397499 | 0.589 | 0.88 | 1.98E−113 |
| ZFP36L2 | −0.7736322 | 0.872 | 0.976 | 2.83E−101 |
| MAL | −0.7589447 | 0.05 | 0.357 | 1.40E−97 |
| JUNB | −0.9204162 | 0.796 | 0.95 | 1.38E−94 |
| EEF1B2 | −0.7334218 | 0.669 | 0.875 | 1.08E−91 |
| TOMM7 | −0.7172297 | 0.588 | 0.833 | 2.89E−86 |
| PNRC1 | −0.6053593 | 0.799 | 0.94 | 8.36E−85 |
| EEF1G | −0.6835015 | 0.706 | 0.88 | 2.88E−84 |
| PLP2 | −0.7767395 | 0.274 | 0.599 | 1.46E−81 |
| CXCR4 | −0.8731744 | 0.719 | 0.896 | 3.18E−81 |
| PFDN5 | −0.6746137 | 0.639 | 0.852 | 7.12E−81 |
| ANXA1 | −0.8938157 | 0.65 | 0.882 | 2.45E−80 |
| TUBA1A | −0.8590816 | 0.417 | 0.711 | 5.11E−79 |

To better understand the difference in phenotype of effector cells specifically within the vaccine-generated neoantigen-specific CD4+ T effector cell (tetramer+) and other effector cell (tetramer−) populations, all effector cells were identified as being CD45RO+CD45RA-CD62L^{lo}CCR7^{lo}, and subject to unsupervised clustering (FIGS. 11D and 11E). This analysis revealed 5 unique clusters, with the tetramer− cells falling mainly in clusters 1 and 2, and tetramer+ cells falling mainly in clusters 0, 3 and 4 (FIGS. 11E and 11F). Expression profiles of each cluster revealed that tetramer− effector cells demonstrate an effector memory phenotype (TCF7, IL7R, CCR7), whereas the tetramer+/vaccine specific CD4+ T effector cells demonstrate activation/exhaustion (PDCD1, ICOS, TIGIT, TOX) or cytotoxic (PFR1, GNLY) phenotypes (FIG. 11G).

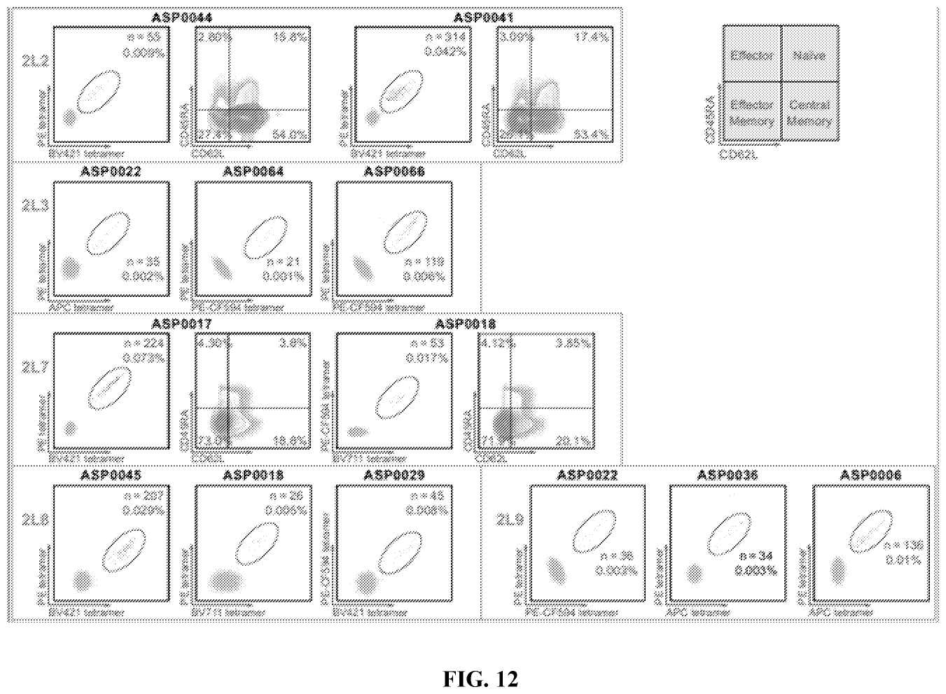

Additional analysis of CD4+ neoantigen-specific T cell responses was performed using a combination of MHC Class II multimer staining and phenotypic analysis by flow cytometry for 10 patients (FIG. 12). Overall, the flow analysis was consistent with the CITEseq analysis described in FIGS. 6A, 6B and 6C, in which the majority of neoantigen-specific CD4+ T cells demonstrate an effector phenotype when compared to tetramer− CD4+ T cells.

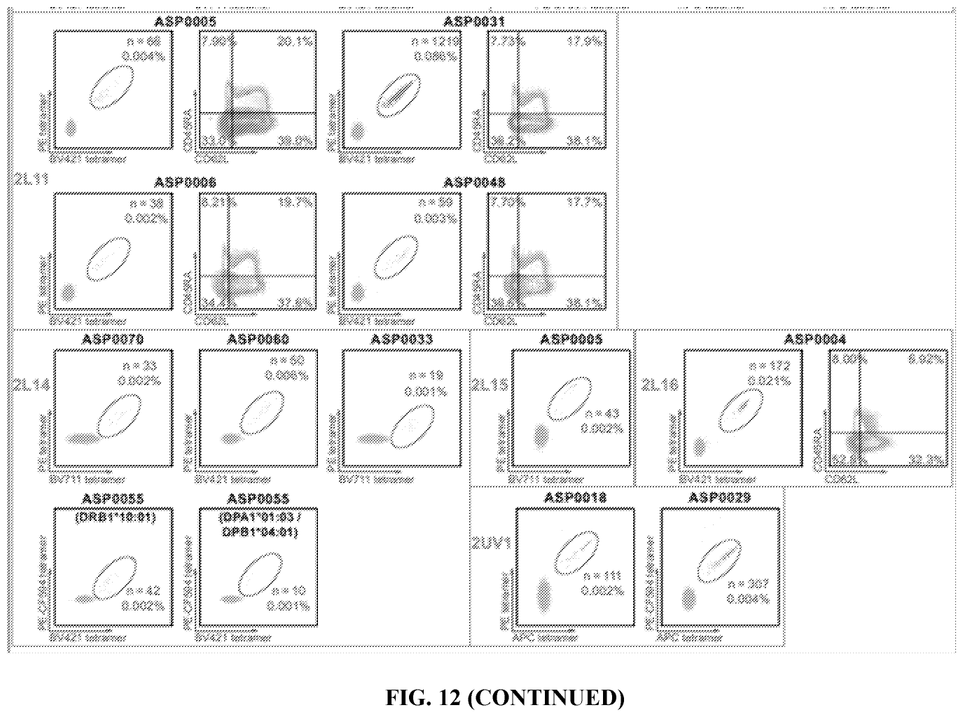

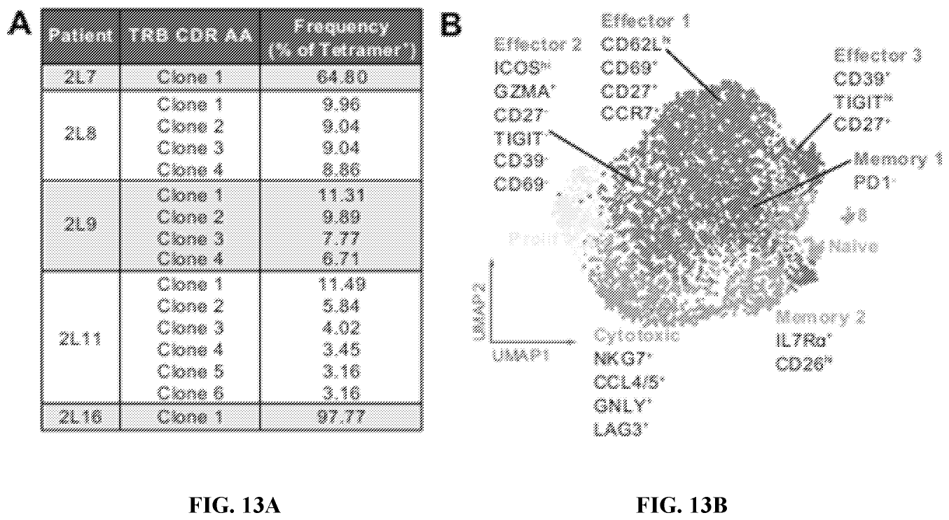

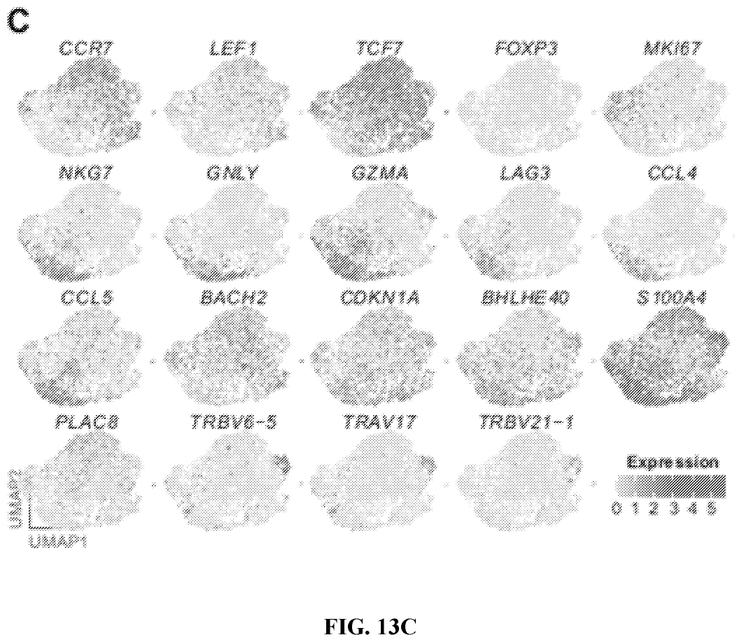

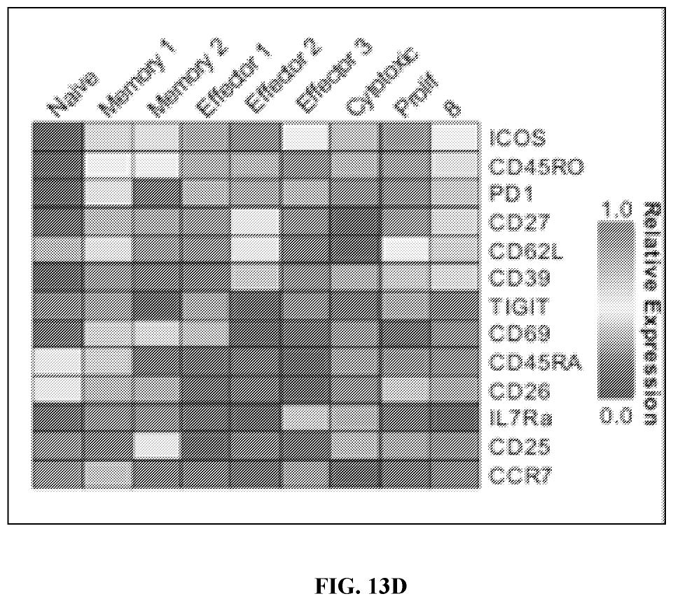

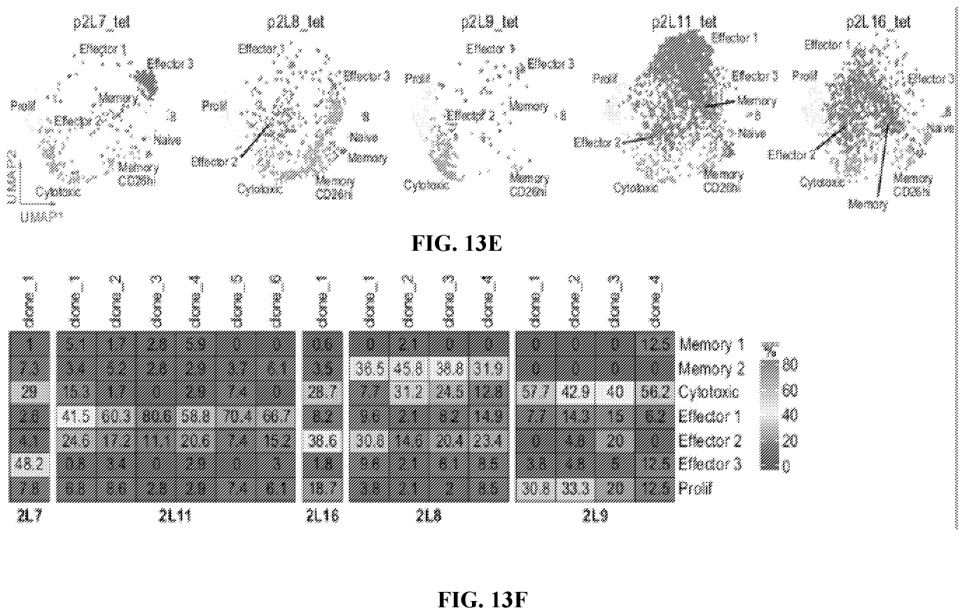

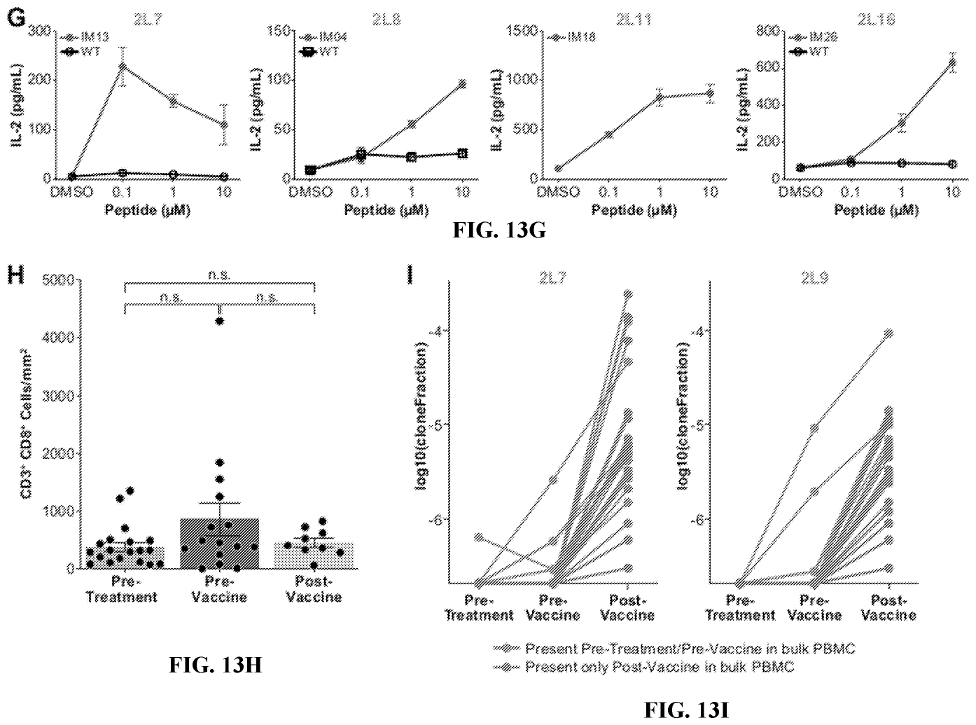

Clustering analysis of only the tetramer+ CD4+ T cells was performed to determine if there are phenotypic differences in the neoantigen-specific CD4+ T cell clones both within an individual patient, as well as between patients (FIG. 13A). Markers representative of each identified cluster and T cell phenotype are shown in the corresponding uniform manifold approximation and projection (UMAP) (FIG. 13B). Gene and protein expression profiles used for clustering analysis are shown in FIGS. 13C and 13D respectively. The abundance of each phenotype observed on a per-patient level is shown in FIG. 13E. Further, the phenotypes of T cells belonging to the most abundant TCR clones coming from the top 30% of each patient's tetramer+ TCR repertoire were assessed. Consistency in phenotypes were observed across clones within a patient, but varying phenotypes when comparing across patients. (FIG. 13F). In order to confirm specificity of the CD4+ TCR clones to the predicted peptide/Class II allele combination, four of the top TCRs identified from each single-cell TCR sequencing result across four patients were validated by cloning the individual TCR into the CD4+ Jurkat cell line, followed by co-culture with patient-matched antigen presenting cells (APCs) loaded with either the mutated neoantigen peptide corresponding to the vaccine or the relevant wild-type peptide. Cloned TCRs for patient 2L7, 2L8 and 2L16 (recognizing the vaccine peptides IM13, IM04 and IM26 respectively) resulted in secretion of IL-2 only in response to the mutated neoantigen peptide, and not the corresponding wild-type peptide (FIG. 13G). No wild-type peptide was available as a control for patient 2L11, in which the vaccine peptide (IM18) targets a frameshift mutation (FIG. 13G).

To validate that the neoantigen-specific tetramer+ CD4+ T cells being observed were present in the patient's peripheral blood only after vaccination, bulk TCR sequencing of CD3+-enriched patient PBMCs were performed at the pre-treatment, pre-vaccine, and post-vaccine timepoints and quantified the abundance of the TCR clones covering the top 30% of the repertoire in concordance with the single-cell TCR sequencing discussed previously (FIG. 13A). Each of these dominant clones only were detected at the post-vaccination timepoint (FIG. 6F). It was not possible to assess any of the top 4 clones for patient 2L8 in the bulk TCR sequencing due to lack of available patient material. Additionally, immunohistochemical analysis of longitudinal tumor biopsies obtained at pre-treatment, pre-vaccine and post-vaccine timepoints was performed where available, revealing a significant increase in the presence of CD4+ T cells at the post-vaccine timepoint when compared to the pre-treatment timepoint (FIGS. 6G and 6H). This increase was observed only in CD4+ T cells and not in CD8+ T cells (FIG. 13H). The increase in CD4+ T cells was also seen at the pre-vaccine time point and continued to increase at the post-vaccine time point. Representative images from the three timepoints from patient 2L7 show a marked increase in CD4+ T cell infiltration into the tumor following the initiation of treatment in both the pre-vaccine and post-vaccine timepoints (FIG. 6H). To address if vaccine-induced T cells can be found in the post-treatment biopsy, the TCR clones were searched that were exclusively found in the post-vaccine biopsy of patient 2L7 and 2L9 and compared the presence of these clones in the periphery across the three time points of pre-treatment, pre-vaccination, and post-vaccination. This identified two distinct sets of clones, ones that expanded upon vaccination in the periphery (FIG. 13I, red) and ones that were detected exclusively at the post-vaccination timepoint in the periphery (FIG. 13I, blue), suggesting vaccine-induced T cells in the periphery can traffic to the post-vaccination tumor. Although the specificities of these tumor penetrating clones are currently unknown, a majority of these clones is presumed to be induced by the vaccine due to their expansion/detection only upon vaccination.

### Discussion:

In this report of a Phase I study in non-squamous NSCLC, the utility of targeting neoantigens in the context of immune checkpoint and chemotherapy combination as first-line treatment was further tested. For the majority of NSCLC patients without a targeted therapy option, the approval of chemotherapy in combination with checkpoint blockade has ushered in an age of immunochemotherapy that resulted in a significant improvement in OS benefit compared to OS achieved with the chemotherapy regimen alone. Despite these advances, the median OS of metastatic NSCLC still remains less than 3 years (Wang, Herbst and Boshoff, 2021) warranting the need to improve upon the current standard of care. This instant report of personalized neoantigen vaccine in this setting evaluates the safety, immunogenicity, and potential clinical benefit in this patient group.

The clinical data from this study indicate that a personalized neoantigen-based vaccination strategy is feasible and safe as first-line treatment in NSCLC in combination with standard of care chemotherapy plus pembrolizumab. The single arm design of the trial does not allow attribution of clinical responses specifically to the vaccine since it was administered in combination with anti-PD-1 and chemotherapy. A study that could serve as a historical comparator is KEYNOTE 189 (Gandhi et al., 2018; Gadgeel et al., 2020). However, this study could not be directly compared to our study given the differences in the patient populations and the treatment regimens. Patients in the KEYNOTE 189 study received maintenance chemotherapy while patients in the NT002 study proceeded to get NEO-PV-01 alone after the initial four cycles of chemotherapy and checkpoint inhibitor. In addition, the NT002 vaccinated study cohort had a higher ratio of female to male patients and lower relative tumor PD-L1 expression, each of which can be associated with inferior clinical outcome to combination of immunotherapy with chemotherapy (Conforti et al., 2018; Gandhi et al., 2018).

Approximately 45% of the enrolled patients were unable to proceed to vaccination due to progressive disease, the inability to manufacture vaccine due to low tumor content in the biopsy or insufficient mutations leading to a low number of high-quality neoantigens. Steps to mitigate such failures would include improved vaccine production timelines, evaluation of TMB using some of the emerging technologies from cell-free DNA and using a vaccine format such as mRNA that can be faster to manufacture and administer as well as overcome the solubility limitation of synthetic long peptides.

With respect to immunogenicity of the personalized vaccine in context of immunochemotherapy, it was encouraging to see that despite receiving an immunosuppressive chemotherapy regimen prior to vaccination, a robust immune response was generated to the vaccine in all patients analyzed, with both CD4+ and CD8+ responses being generated. Interestingly, a large majority of the vaccine-induced cytotoxic responses were CD4+ T cells. This is in contrast to our previous study where both CD4+ and CD8+ cytotoxic T cells were observed to be generated in response to NEO-PV-01 vaccination (Ott et al., 2020) in combination with nivolumab alone. The differences observed with the previous study could be attributed to the differences in treatment combination or can be since this was a cohort of exclusively first-line NSCLC cancer patients. Additionally, robust MHC Class II expression was observed in the tumor microenvironment of this patient cohort, particularly at the post-vaccination timepoint. This suggests the presence of a microenvironment in favor of developing CD4+ T cell responses. Skewing towards CD4+ T cell responses has been reported previously in the setting of neoantigen vaccine as well (Ott et al., 2017; Sahin et al., 2017b).

The use of single-cell sequencing technologies and multiparametric flow-based analysis has further enabled the detailed characterization and definition of gene expression patterns associated with such cytotoxic CD4+ T cells. Our data demonstrates the generation of such effector and cytolytic CD4+ T cells upon vaccination with a personalized vaccine. Of note, the CD4+ neoantigen T cells generated in the study were not of the Treg phenotype (did not express Foxp3) and instead had a distinct effector cytotoxic phenotype. In summary, our data suggests that vaccine-induced CD4+ T cells generated in the context of immunochemotherapy have the properties of T cells that could mediate tumor cell killing. Additionally, the increase in CD4+ T cells and not CD8+ T cells post-initiation of treatment emphasizes a critical role for CD4+ T cell compartment in tumor control.

Another aspect of our study is the generation of epitope spread responses in multiple patients. Of particular interest are the epitope spread responses seen toward common driver mutations KRAS G12C and KRAS G12V across multiple patients. Pre-existing immune responses to KRAS G12V and KRAS G12D mutation have been reported in a cohort of patients from colorectal cancer and a NSCLC patient previously. We report here epitope spread responses to KRAS G12C and G12V mutations generated post-treatment with NEO-PV-01 in combination with anti-PD-1 and chemotherapy. Identification of such immune responses in our study provides additional data on KRAS mutation-specific T cells that can be further developed for T cell or vaccine-based therapies.

A limitation of our study is its single arm design. Since the vaccine was administered in combination with anti-PD-1 and chemotherapy, the clinical responses, epitope spread, and immunogenicity cannot be solely attributed to the vaccine alone. Randomized trials of neoantigen therapy in combination with anti-PD-1 and chemotherapy vs immunochemotherapy alone will be necessary to confirm these findings and determine the specific contribution of the personalized vaccine.

In summary, this study demonstrates the safety and feasibility of a personalized neoantigen-based vaccine in combination with anti-PD-1 and chemotherapy as first-line treatment in NSCLC. The combination leads to robust generation of immune responses possessing a phenotype associated with tumor cell killing, with a predominance of CD4+ T cell responses. Also observed was epitope spread to a common driver mutation and additional neoantigens. Identification of potential tumor biomarkers holds promise for selecting patients in further randomized trials. These trials will be required to further refine the use of personalized neoantigen vaccine in combination with immunochemotherapy as first-line treatment in NSCLC.

### Example 2. Methods Used in Phase 1b Study

### Study Design

Details of the study can be found in the attached clinical study protocol NT-002 and patient demographics can be found in Table 1.

The clinical sites that enrolled patients were Dana Farber Cancer Institute, Sarah Cannon Research Institute, University of California Los Angeles, and Washington University.

The primary objective of this phase 1b trial was to evaluate the safety of administering NEO-PV-01 with pembrolizumab/chemotherapy in untreated patients with advanced or metastatic non-squamous NSCLC. Secondary objectives included determination of antitumor activity, assessed by RECIST objective response rate, clinical benefit rate, duration of response, response conversion rate, progression free survival, and overall survival.

The study employed multiple exploratory objectives to characterize immune responses to NEO-PV-01 regimen in this population. Vaccine-induced responses were evaluated by assessment of antigen-specific CD8+ and CD4+ T-cell responses in peripheral blood during and following vaccine administration. Correlation of patient responses including prolonged PFS with biomarkers in peripheral blood and tumor was conducted as an exploratory objective; assessments included tumor programmed death ligand (PD-L1) expression and abundance and phenotypes of tumor infiltrating lymphocytes. Patient samples used across the various assays such as in assaying ctDNA, Tumor PD-L1, CD4+/CD8+ cells in tumor at week 0, MHC class II GEX (tumor, week 0), bulk TCR sequencing from tumor, ELISpot analysis at week 10 and/or 20, WT vs. mutant at week 20, ELISpot persistence at week 52, cytotoxicity at week 20, epitope spread at week 10 and 20, epitope spread persistence at week 52, epitope spread—comparison of WT vs mutated at week 20, MHC class II multimers at week 10 or 20, and MHC class II multimer as well as activation at week 20, ssRNA sequencing, CITE or TCCR analysis at week 20; bulk peripheral PBMC TCR sequencing at week 0, 10 and 30; CD4+/CD8+ tumor IHC and TCR cloning.

### Treatment Regimen

NEO-PV-01 is a personalized cancer vaccine consisting of up to 20 synthesized peptides of approximately 14 to 35 amino acids in length that are derived from an individual patient's mutated tumor DNA. The NEO-PV-01 drug product is administered following mixing with adjuvant poly-ICLC. For each patient, the peptides in poly-ICLC are divided in up to 4 pools for injection subcutaneously, rotating in extremity or flank site administrations.

In this phase 1b study, NEO-PV-01 therapy is administered following four 3-weekly cycles of a chemotherapy+pembrolizumab treatment regimen. Pembrolizumab was administered as a 200 mg IV infusion on cycle day 1 every 3 weeks. Carboplatin (AUC 5) and pemetrexed 500 mg/m2 were also given on day 1 of each treatment cycle. Patients were required to receive all 4 pembrolizumab+chemotherapy cycles prior to vaccination. In instances where these treatments were held, NEO-PV-01 initiation was also delayed until all 4 cycles were complete.

All patients underwent radiographic evaluation at study week 8 to assess disease response to initial chemotherapy+pembrolizumab and had a confirmatory assessment at week 12 to document disease status prior to initiation of NEO-PV-01 vaccination.

NEO-PV-01 is administered on a schedule of 5 priming doses days 1 and 4 of study week 12 and then weekly in study weeks 13, 14, and 15. Two additional NEO-PV-01 administrations are provided as booster dosing on study weeks 19 and 23 to complete vaccine administration and the study primary treatment phase.

Pembrolizumab treatment was maintained on its every 3 week schedule during the week 12-24 vaccination period and then from weeks 24-103 in the absence of progressive disease, initiation of an alternate antineoplastic therapy, intolerable toxicities, withdrawal of consent, or death.

### Eligibility

Patients eligible for study participation were those with histologically confirmed, locally advanced or metastatic non-squamous NSCLC and no prior therapy for their advanced disease. Other key inclusion criteria include written informed consent for study participation, age ≥18 years, ECOG performance status 0 or 1, at least 1 site of RECIST measurable disease, disease accessible for on treatment tumor biopsy, and acceptable screening laboratory values within 30 days prior to treatment including hematology: absolute neutrophil count (ANC) ≥1.5×10³/mL, platelet count ≥100×10³/mL, hemoglobin >9 g/dL, chemistry: serum creatinine ≤1.5× upper limit of normal (ULN) or creatinine clearance (CrCl) ≥40 mL/min/1.73 m², aspartate aminotransferase (AST) and alanine aminotransferase (ALT) ≤2.5×ULN or ≤5×ULN with liver metastases, total bilirubin ≤1.5×ULN or direct bilirubin ≤ULN for patients with total bilirubin levels >1.5×ULN, and PT, PTT both <1.5×ULN unless on anticoagulation therapy.

Patients eligible for study participation did not meet any of the following major exclusion criteria, including receipt of any prior systemic cancer therapy for advanced or metastatic NSCLC, receipt of any investigational agent or study therapy within 4 weeks of first study treatment, receipt of radiation therapy to the lung greater than 30 Gray <6 months prior to study treatment, known CNS metastases unless clinically stable >4 weeks, receipt of non-oncology vaccine therapy during and up to 8 weeks following the period of NEO-PV-01 administration, active autoimmune disease requiring systemic treatment within the last 2 years except for physiologic corticosteroid replacement therapy, interstitial lung disease, active pneumonitis or history of pneumonitis requiring corticosteroid therapy, unstable angina or congestive heart failure, active infection requiring treatment, active hepatitis B, C, or history of HIV, or history of invasive malignancy unless disease free >2 years, Patients with anaplastic lymphoma kinase (ALK) translocations or epidermal growth factor receptor (EGFR) mutations must have received prior treatment with ALK or EGFR inhibitors, respectively.

A complete list of inclusion and exclusion criteria is provided in the NT-002 study protocol (Appendix).

### Clinical Assessments

Safety assessments conducted during the primary treatment phase included adverse event collection through patient reported symptoms, symptom-directed physical examination, and vital sign and safety laboratory assessments. Adverse events were monitored throughout the study from time of first dose of pembrolizumab (Cycle 1, Day 1) through study week 103 or 30 days after the last pembrolizumab treatment, whichever occurred first. Serious adverse events (SAEs) were reported from the time of signing the patient informed consent through study week 103 or 90 days after the last dose of pembrolizumab, whichever occurred first.

Radiographic assessments to evaluate response to study treatment were conducted at weeks 8 and 12 prior to NEO-PV-01 administration, at study weeks 24, 36, 51, 63, 75, 87, and 99.

### Patient Samples

Patient samples collected on the study and analyzed across the various assays such as assaying ctDNA, Tumor PD-L1, CD4+/CD8+ cells in tumor at week 0, MHC class II GEX (tumor, week 0), bulk TCR sequencing from tumor, ELISpot analysis at week 10 and/or 20, WT vs. mutant at week 20, ELISpot persistence at week 52, cytotoxicity at week 20, epitope spread at week 10 and 20, epitope spread persistence at week 52, epitope spread—comparison of WT vs mutated at week 20, MHC class II multimers at week 10 or 20, and MHC class II multimer as well as activation at week 20, ssRNA sequencing, CITE or TCCR analysis at week 20; bulk peripheral PBMC TCR sequencing at week 0, 10 and 30; CD4+/CD8+ tumor IHC and TCR cloning. 38 patients were enrolled on the study. Peripheral blood mononuclear cells (PBMCs) samples for comprehensive immune system monitoring were obtained from leukaphereses performed up to 7 days prior to initiation of NEO-PV-01 vaccination, 7 days (Week 20) following the first NEO-PV-01 booster vaccination and then at study week 52. Additional immune monitoring assessments were performed from 80 mL peripheral blood samples obtained prior to study treatment, and study weeks 6, 14, 16, 24, 36, 48, 63, 75, 87, and 99. Patient PBMCs were processed and stored as described in Ott et al.

Tumor samples (surgical and core-needle biopsies) were obtained at pre-treatment, pre-vaccine (week 10-12) and post-vaccine (week 24) timepoints. Samples were processed and sequenced as described in Ott et al. Pre-treatment biopsies were used for generation of NEO-PV-01, and advanced for sequencing as described previously (Ott et al., 2020).

### Generation of NEO-PV-01

### Whole Exome Sequencing

As previously described (Ott et al., 2020). WES (library protocol: ACE™ Cancer Research Exome, Personalis, Menlo Park, CA) and RNA-Seq (library protocol: ACE™ Cancer Research Transcriptome, Personalis, Menlo Park, CA) libraries were sequenced using Illumina HiSeq in a CLIA/CAP accredited laboratory (Personalis, Menlo Park, CA). WES was conducted on a tumor sample and a normal blood sample per patient (depths: 163-335 reads and 80-137 reads, respectively); RNA-Seq was conducted on tumor samples only (depth: 37-309 reads). The HLA-A, HLA-B, and HLA-C genotype of each patient was determined by amplifying informative exons by polymerase chain reaction (PCR) using locus-specific primers. Dye-terminator sequencing fragments from the PCR fragments were analyzed on a capillary sequencer to determine nucleotide sequences for each haplotype (Blood Center of Wisconsin, Milwaukee, WI).

### Alignment and Mutation Calling

All alignment, mutation calling and filtering, and gene expression quantification was done in accordance with methods described previously (Ott et al., 2020). In the event that an adequate RNA library could not be prepared, methods described previously were implemented (Ott et al., 2020). All computational analysis steps were conducted using an automated analysis pipeline hosted on a cloud computing platform (Seven Bridges Genomics, Charlestown, MA). Tumor and germline WES and tumor RNA-Seq FASTQs were aligned to the human genome (Gencode V19) using the BWA-MEM (version 0.7.13) (Li and Durbin, 2009) and STAR (version 2.5.1.b), respectively. All alignments were post-processed with the GATK 3.5 workflow (McKenna et al., 2010) including GATK Indel Realigner, GATK Base Recalibrator, and Picard Mark Duplicates (version 1.140). ConTest (Cibulskis et al., 2011) was used to confirm that all three samples originated from the same individual. Somatic variants were called on the basis of tumor and normal WES using an ensemble of seven different mutation calling algorithms: VarDict (version 1.4.6) (Lai et al., 2016), Strelka (version 1.0.15) (Saunders et al., 2012), VarScan2 (version 2.3.9) (Koboldt et al., 2012), Mutect2 (from the GATK version 3.5 bundle) (Cibulskis et al., 2011), Atlas Indel2 (version 1.4.3) (Challis et al., 2012), Seurat (version 2.6) (Christoforides et al., 2013), and Platypus (version 0.8.1) (Rimmer et al., 2014). The three sequencing datasets were then realigned using HaplotypeCaller (from the GATK version 3.5 bundle), specifying the candidate mutations (union of the seven call sets) as known variants. Finally, the variants were filtered according to the following features: the level of read support in the tumor WES data, the presence of variant reads in the normal WES data, read orientation bias, adequacy of coverage in the normal WES sample, the presence of neighboring (+/_30nt) variants (somatic or germline), and read quality bias observed in mutation-supporting reads. RNA-Seq expression levels of all genes and transcripts were quantified in transcripts per million (TPM) using RSEM (version 1.2.31) (Li and Dewey, 2011). The overall expression of each somatic variant was calculated as the product of the RSEM-derived transcript expression (summing across all overlapping protein-coding transcripts) and the fraction of RNA-Seq reads supporting the variant. Variants with zero supporting RNA reads were still considered as valid mutations (and counted toward tumor mutation burden) but were not considered for inclusion in vaccine. RNA-Seq was additionally processed using STAR-Fusion (version 2.5.1.b) to identify transcript fusions (requiring both junction support and spanning read pairs). In the event that an adequate RNA library could not be prepared, fusion calling was skipped, and reference expression was obtained from TCGA samples with high tumor purity (as assessed by Absolute; five samples per tumor type) (Carter et al., 2012). Since these patients lacked RNA-Seq as a filter against false positive mutation calls, their somatic variants were required to have a higher level of read support in the tumor WES and to have been identified by more than one mutation caller.

### Vaccine Peptide Selection

Patients with at least 50 non-synonymous point mutations and/or gene fusions were advanced to the vaccine design step. The vaccine peptides, also referred to as immunizing (IM) peptides, were identified by selecting 6 peptides on the basis of HLA-I presentation scores (also referred to as epitope quality scores, see below), 2 peptides based on high expression, and 2 peptides based on highly expressed frameshifts, iteratively until the full roster of 30 peptides was completed. The latter two epitope classes were required to have expression levels >10 TPM and >5 TPM, respectively (if none were available, the slots were ceded to HLA-I selections). Generally, each mutation was targeted by only one vaccine peptide and excluded from subsequent selections unless the span of novel sequence generated could not be covered by a single peptide (as can happen with frameshifts) or if the likelihood of manufacturing success was questionable for the first peptide per known synthesis constraints. The trade-off between synthesis constraints and immune-related scoring further dictated the relative length of the vaccine peptides (allowable range: 14-35 amino acids) and whether they were centered or shifted with respect to the site of mutation.

HLA-I presentation scores were determined based on a logistic regression that considered binding predictions, allele-specific expression, and proteasomal cleavage potential. Binding predictions were calculated as the weighted average of NetMHCpan-3.0 percent rank (Nielsen and Andreatta, 2016) and a neural network trained on mass spectrometry data using methods described previously (Abelin et al., 2017) with respective weights of 20% and 80%. Proteasomal cleavage potential was also calculated using a neural network trained on mass spectrometry data using methods described previously (Abelin et al., 2017). The score of a candidate vaccine peptide was determined by summing all the relevant peptide-allele combinations (epitopes lengths 8-12; HLA-A HLA-B, and HLA-C alleles).

Secondary characteristics were used to up-weight and down-weight candidate epitopes but were purposely tuned to have less importance than the above-mentioned factors. Thus, all other parameters being equal, the selection algorithm favored more clonal epitopes (higher mutant allele fraction) over sub-clonal epitopes, frameshifts over point mutations, oncogenes over passenger genes, and peptides with good manufacturing scores over those with poor predicted manufacturability. Our scoring system did not consider the relative binding scores of wild-type peptides or similarity to known pathogen sequences (Luksza et al., 2017).

### GMP Peptide Manufacturing

The GMP grade peptides of NEO-PV-01 were manufactured as described previously (Ott et al., 2020). The GMP grade peptides of NEO-PV-01 were synthesized using solid-phase peptide synthesis (SPPS) on automated parallel peptide synthesizers. 9-fluorenylmethyloxycarbonyl (Fmoc) chemistry was employed. After the assembly of the peptide chains, the peptides were removed from the resins using cleavage cocktails containing trifluoroacetic acid and scavengers. The peptides were precipitated and washed with ether. After drying, the crude peptides were purified on automated preparative high-performance liquid chromatography (prep HPLC) systems with ultraviolet (UV) and mass spectrometry (MS) detectors. The prep HPLC fractions were analyzed using ultra-performance liquid chromatography (UPLC)-UV/MS systems to determine molecular weights (MW) and purities. The fractions containing the target peptides and desired purities were lyophilized to solid powders with the final purities equal to or higher than 95%. Up to 20 peptides per vaccine were formulated in a buffered aqueous solution containing 4% DMSO in isotonic dextrose and mixed into up to four pools, each containing up to five peptides. The pooled peptide solutions were filtered through 0.22 mm filters for sterilization. The vaccines were analyzed for identity (molecular weights based on UPLC-UV/MS analysis), sterility, endotoxin, and strength before their releases from the GMP manufacturing site. The amino- and carboxyl-termini of the peptides are free amines and carboxylic acids respectively, without structural modifications.

### Peptide Synthesis for Immunological Assays

The peptides used for in vitro immune response studies were synthesized as described in Ott et al. The peptides for in vitro immune response studies were synthesized using SPPS on computer-controlled high-throughput peptide synthesizers. The peptides were assembled on resins by employing Fmoc chemistry. The chemical cleavages of peptides from the resins were performed by using TFA solution containing scavengers followed by peptide precipitation and washes with diethyl ether. The assay peptides (ASP) were purified on automated prep HPLC systems equipped with UV and MS detectors. The prep HPLC fractions of each peptide that met the molecular weight (MW) and purity criteria based on UPLC-UV/MS analysis were pooled and dried using either parallel centrifugal evaporators or lyophilizers. The epitope peptides (EPT) were resuspended in acetonitrile and water and dried on a lyophilizer after diethyl ether washes and analyzed for required purity and MW by using UPLC-UV/MS. Assay peptides (ASP) were 13-15 amino acids and overlapped by at least 9 amino acids to cover the immunizing peptide (IM) sequence or were 8-11 amino acids and predicted to bind class I (Ott et al., 2017). All assay peptide sequences are provided in Table S8.

Personalized ctDNA Assays

Design and application of personalized and tumor-informed ctDNA (multiplex-PCR, next-generation sequencing) assays was conducted with blinding to clinical data by Natera. Paired tumor and peripheral blood WES data were used to identify and design the Signatera assay with 16 tumor-specific somatic single nucleotide variants (SNVs) for each patient. Additionally, another custom assay consisting of 16 tumor-specific SNVs was designed from our BioNTech's list of 30 prioritized high-quality patient-specific neoepitopes based on designability and other internal bioinformatics algorithmic criteria (Reinert et al., 2019; Coombes et al., 2019; Christensen et al., 2019). Given that these algorithms were developed independently, there is a potential of variants to overlap between the two assays. For the first Signatera 16-plex assay, SNVs present in the tumor but absent in the germline were used to select the 16 targets, with prioritization based on multiple factors, including the observed variant allele frequency in the tumor tissue and the associated background noise profile in the plasma. The multiplexed targeted PCR was conducted followed by amplicon deep sequencing on an Illumina platform. Variant allele frequencies were determined for each of the target mutations. The plasma sample was considered ctDNA-positive on observing at least two or more variants. Absolute ctDNA levels were then calculated for ctDNA-positive plasma samples (MTM per ml) by normalizing variant allele frequencies observed by the plasma volume used for each sample. As described previously, MTM per ml was calculated from all tested targets, including undetected targets (Bratman et al., 2020).

### Detection of Neoantigen-Specific Immune Responses

Neoantigen-specific immune responses were detected either ex vivo or after 5-day stimulation as described previously (Ott et al., 2020). For ex vivo assays, patient PBMCs were rested overnight at 2×10⁶ cells/mL in X-Vivo media (Lonza) supplemented with 1% penicillin/streptomycin (GIBCO). For neoantigen pre-exposure assays, patient PBMCs were rested overnight and cultured with individual peptides (2 mM) or pooled peptides (2 mM per peptide) for 5 days at a density of 5×10⁶ cells/ml in a 24-well plate. On day 3 of culture, half the well volume was replaced with fresh media. All immunizing peptides were tested in both the ex vivo as well as in the 5-day stimulation with the neoantigen peptide assay formats. Immunizing peptides are referred to as IM followed by the peptide number.

### IFNγELISpot Assay

IFN-γ ELISpots were performed as described previously (Ott et al., 2020). IFNγ ELISpot assays were performed using 96-well MultiScreen Filter Plates (Millipore) and the Ready-Set-Go! Human IFNγ ELISpot Kit (Invitrogen) according to manufacturer's instructions. Plates were coated overnight at 4° C. with anti IFNγ Capture Antibody diluted in 1× Coating Buffer, washed with 1× Coating Buffer and blocked with X Vivo media (Lonza) containing 1% penicillin/streptomycin (GIBCO) for 1 h. For ex vivo ELISpots, PBMCs were plated in triplicate with 1×10⁶ cells per well. For cells that were pre-exposed to peptide, cells were washed and plated in triplicate with 1×10⁵ cells per well. Peptides were added to ELISpot wells at 2 mM per peptide. Each plate included a healthy donor positive and negative control with the CEF viral peptide pool (JPT) and Cell Stimulation Cocktail (Life Technologies) to confirm reagent performance. Plates were incubated overnight at 37° C. Plates were washed 3 times using PBS with 0.05% Tween-20 and detection antibody diluted in 1×ELISpot diluent was added to wells for 2 h. After washing 3 times with PBS with 0.05% Tween-20, Avidin-HRP was diluted in 1×ELISpot diluent and added to wells for 45 min. Plates were washed with both PBS with Tween-20 and PBS 3 times. AEC substrate-chromogen (BD Biosciences) was then added for 20 min. Plates were rinsed with deionized water 3 times and allowed to dry at room temperature overnight. Spots were imaged and enumerated using an Immunospot analyzer (Cellular Technology Limited). Responses were characterized as positive if spot-forming cell count detected was at least 10 spots over the DMSO control and +3 standard deviations and confirmed in repeat experiments.

### Characterization of CD4+/CD8+ T Cell Responses

CD4+ and CD8+ T cell response were characterized as described previously (Ott et al., 2020). CD3+ T cells were isolated from patient PBMCs by negative selection using the Pan T cell isolation kit (Miltenyi). Both the CD3+ and CD3− populations were collected, washed, and counted after isolation. The CD3− population was used as APCs (antigen presenting cells) for this assay. The CD3+ population then underwent CD4+ positive isolation using CD4+ microbeads (Miltenyi). Both the CD4+(positive-selection) and CD8+(negative-selection) T cells were collected, washed, and counted. A co-culture containing APCs and either CD4+ or CD8+ cells at a ratio of 3:1, 2:1, or 1:1 were plated in a 96-well flat bottom polystyrene plate for 24-48h. Peptide was added directly to wells, in triplicate, at 2 mM per peptide. If the T cells were pre-exposed to neoantigen peptide for 5-6 days, CD3− APCs were isolated from fresh patient PBMCs using CD3 microbeads (Miltenyi). Supernatants were collected at the end of the coculture and frozen at −80° C. until use. The Meso Scale Discovery (MSD) U-Plex system was used to detect 10 distinct analytes from the coculture supernatants. The analytes measured were: IL-1b, IL-2, IL-6, IL-9, IL-13, IL-15, IL-17a, IFNγ, and TNF-a. MSD 10-plex plates were coated with the linker-antibody solution for 1h with shaking at room temperature. The plates were then washed 3 times with PBS with 0.05% Tween-20 on a BioTek plate washer. Supernatant and standards were diluted 1:2 and added to the plate for 1h with shaking at room temperature. Plates were washed again with PBS with 0.05% Tween-20 before the addition of the detection antibody solution for 1h. Plates were washed with PBS with 0.05% Tween-20 and 2× Read Buffer (MSD) was added to the plates for detection on the MSD SECTOR S 600 instrument. Concentration in pg/mL was calculated by the MSD Discovery Workbench software by comparing luminescent signal of the patient samples against the known standard curve. CD4+ and CD8+ positive responses were determined separately based on the corresponding DMSO controls. A positive signal was determined by an IFNγ signal 1.5-fold higher than the DMSO control.

### CD107a Mobilization Assay

Detection of CD107a mobilization on both CD4+ and CD8+ T cells was performed using flow cytometry as described previously in (Ott et al., 2020). For ex vivo detection, patient PBMCs were recalled with 2 mM peptide or DMSO for 6 or 24h. For patient PBMCs pre-exposed to neoantigen peptide, CD3− APCs were isolated from fresh PBMCs using CD3 microbeads (Miltenyi) and co-cultured with CD3+ T cells at a T cell:APC ratio of 2:1 and recalled with 2 mM peptide or DMSO for 6 or 24h. Anti-CD107a antibody was added 6h prior to the end of co-culture. GolgiStop/Plug (BD Biosciences) were added 4h prior to the end of co-culture. Subsequently, cells were stained with cell surface antibodies at 4° C. for 30 min, followed by fixation/permeabilization with Fixation and Permeabilization Solution (BD Biosciences), and subsequently stained with antibodies against intracellular proteins at 4° C. for 30 min. Cells were stored in FACS buffer at 4° C. until acquisition on a BD LSR Fortessa instrument. Gating was performed for IFNγ and CD107a based on FMO stained controls.

### MHCII Protein Expression, Purification, and Peptide Exchange

Signal peptides and ectodomains of alpha and beta MHC class II alleles were encoded as fusions to heterodimeric coiled coils in a pcDNA3.4 vector (Thermo Fisher Scientific). Allele-specific placeholder epitope peptides were covalently fused to the N-terminus of each beta allele. Soluble heterodimers of alpha/beta proteins were secreted directly into culture media post-transfection into Expi293F cells (Thermo Fisher Scientific) and purified using HisTrap Excel media (Cytiva). Purified MHC class II alleles were subsequently biotinylated and the placeholder peptide cleaved prior to peptide exchange. Epitope peptides (at 20-fold molar excess to allele) were loaded onto purified MHC II alleles using equimolar recombinant soluble HLA-DM overnight at 37° C.

### Multimer Generation and Staining

Dual-multimer approach was used where each peptide-HLA II allele was conjugated to two different streptavidin fluorophores. Peptide exchanged allele was incubated with fluorochrome-conjugated streptavidin antibodies on ice for 30 minutes in the dark. Biotin was added to block any unoccupied sites on streptavidin fluorophore. Reaction was spun down at 3500 RPM (4° C.) for 10 minutes to remove any aggregates. The conjugated multimer supernatants were used for making a pooled multimer mix.

Patient PBMCs were thawed and subjected to T cell enrichment by using a human Pan T Cell Isolation Kit (Miltenyi Biotec). Isolated T cells were counted and treated with Benzonase and 50 nM Dasatinib for 20 minutes at 37° C. in RPMI media containing 10% FBS. Enriched T cells were centrifuged at 1500 RPM for 5 minutes, washed once with FACS buffer (1×PBS+0.5% BSA) supplemented with 50 nM dasatinib, and plated in a 96-well plate (3×106 cells/well). Cells were resuspended in a pooled multimer mix (Supplementary Table) made in FACS buffer and incubated for 1 hour at 37° C.

### Phenotyping of Multimer+CD4+ T Cells

Phenotyping of neoantigen-specific CD4+ T cells was performed as described previously (Ott et al., 2020). PBMCs were thawed and treated with 0.025 U/ul benzonase and 50 nM dasatinib in X-Vivo media (Lonza) at 37° C. for 20 min. Cells were then stained with MHC Class II tetramers at 37° C. for 1 hr, followed by staining with surface antibodies at 4° C. for 30 min. Cells were fixed at 20° C. for 20 min using the Fixation/Permeabilization Kit (BD Biosciences). Cells were washed and stored in FACS buffer at 4° C. until acquisition on a BD LSR Fortessa instrument.

RNA Extraction from FFPE Tumors and PBMCs

For FFPE tumor blocks, 2-4 scrolls of 20 mM thickness were deparaffinized using heptane and RNA was extracted using the AllPrep DNA/RNA FFPE kit according to the manufacturers' instructions (QIAGEN). PBMCs were thawed and subjected to negative selection using the Pan T Cell Isolation Kit (Miltenyi) according to manufacturer's protocol. T cells were counted, centrifuged for 15 minutes at 300×g then flash frozen and stored as dry pellets at −80° C. RNA isolation was performed using the RNeasy Plus Micro Kit (QIAGEN) on the QIAcube (QIAGEN; Protocol: Purification of total RNA using gDNA Eliminator and RNeasy MinElute spin columns) according to manufacturer's protocol. RNA concentration was measured using the Qubit™ RNA HS Assay Kit (ThermoFischer) according to manufacturer's protocol. Eluted RNA was stored at −80° C.

### Gene Expression Analysis

Using the transcript per million (TPM) quantification of gene expression from the tumor biopsy RNA, we summed the TPM values for all detected HLA-DR, HLA-DP and HLA-DQ per patient. The log-transformed values were plotted against each patient PFS.

### TCR Library Preparation and Sequencing

TCRβ libraries were prepared from isolated RNA using the Long Read iR-Profile Reagent System (iRepertoire) at the iRepertoire headquarters according to manufacturer's protocol. Libraries were sequenced using the MiSeq Reagent Kit v2 300-cycles (Illumina) at the iRepertoire headquarters according to manufacturer's protocol. Throughout the study, the number of samples per pool was designed to maintain equal sequencing depths across samples.

### TCR Repertoire Generation

TCR repertoires were generated by running a licensed copy of MiXCR 3.0.12 on the paired-end raw sequencing fastq files. The parameters included the species specifications (Human, hsa), starting material (RNA), 5′ and 3′ primers (v and c primers, respectively) with no adapters, and searching for either TCRα (tra) or TCRβ chains (trb). TCRα or TCRβ CDR3 clonotypes were filtered by removal of non-functional sequences (out-of-frame sequences or those containing stop codons). Clonal frequency was calculated based on the count for each clone out of the total count.

### TCR Repertoire Diversity/Clonality Analysis

The unique number of nucleotide/amino acid TCRα or TCRβ CDR3s was calculated per sample. Global diversity/clonality coefficients have been calculated as follows:

Gini Coefficient—ranges between 0 (all clones are equally frequent—repertoire diversity) and 1 (frequency dominated by one clone, repertoire clonality). Calculated using the “Gini” function from the “DescTools” R package (S, 2021) (Signorell, A. (2020). DescTools: Tools for Descriptive Statistics® package version 0.99.32).

Normalized Shannon's Entropy—higher values represent higher inequality of the frequencies (Jia et al., 2015; Hanson et al., 2020).

### Single-Cell RNA Sequencing of Neoantigen-Specific CD4⁺ T Cells Using HLA Class II Multimer Staining

Cell surface protein labeling for single-cell RNA sequencing and cell sorting.

After HLA Class II multimer staining, Fc blocking was performed through incubation with human FcR Blocking Reagent (Miltenyi Biotec) for 10 minutes at 4° C. For cell surface protein labeling for sequencing, a mix of 19 TotalSeq™-C antibodies (BioLegend, STAR Methods Table) was added to the multimer-stained cells. Cells were simultaneously labeled with FITC anti-human CD4 (BD), BV711 anti-human CD3 (BD), AF700 anti-human CD8 (BioLegend) antibodies, and LIVE/DEAD™ Fixable Near-IR Dead Cell Stain Kit (Thermo Fisher). After 30-minutes of incubation at 4° C., cells were washed twice in PBS with 1% BSA and sorted using a BD Aria cell sorter (BD Bioscience). For each patient sample, two different CD4⁺ T cell populations were sorted: Tetramer⁻ CD4⁺ (Live CD3⁺ CD4⁺ CD8⁻) and Tetramer⁺ CD4⁺ (Live CD3⁺ CD4⁺ CD8⁻ ASP multimer⁺).

### Single-Cell TCR Surface Epitope and Transcriptome Sequencing

After sorting, labeled cells were resuspended at ˜1×10³ cells/ml in PBS supplemented with 2% FBS and submitted to subsequent single-cell RNA sequencing (scRNA-Seq) processes. Sample processing for single-cell gene expression (GEX), TCR V(D)J clonotypes (VDJ), and cell surface protein expression (CITE) libraries was performed following the manufacturer's protocol (Chromium Next GEM Single Cell 5′ Reagent Kits v2 (Dual Index), 10× Genomics). Briefly, sorted Tetramer⁺ and Tetramer⁻ CD4⁺ T cell suspensions were partitioned into nanoliter-scale Gel Beads-in-Emulsions (GEMs) using a Chromium Controller (10× Genomics). Subsequent Gel Bead-in-Emulsion reverse transcription (GEM-RT) reaction & post clean-up and cDNA amplification were carried out to generate cDNAs for scRNA-Seq libraries. Constructing cDNA libraries for GEX (Library Construction Kit), VDJ (Chromium Single Cell Human TCR Amplification Kit) and CITE (5′ Feature Barcode kit) was performed based on the manufacturer's protocols. All sequencing library quality controls were performed by using Bioanalyzer High Sensitivity DNA Chip Kits with an Agilent 2100 Bioanalyzer (Agilent Technology). Sequencing libraries were quantified using KAPA Library Quantification Kits—Complete kit and LightCycler 480 (Roche).

All libraries were incorporated with Sample Index sequences (Dual Index Kit TN Set A for CITE libraries and Dual Index Kit TT Set A for VDJ & GEX libraries) and sequenced on MiSeq (MiSeq Reagent Kit v2 (300-cycles)) or NovaSeq Si platform (Illumina) depending on sequencing depth. The sequencing parameters were: Read 1 of 26 cycles, i7 Index of 10 cycles, i5 Index of 10 cycles, and Read 2 of 90 cycles.

### Cloning of Neoantigen-Specific TCRs

Lentivirus vectors were generated on 293FT producer cell lines using shuttle plasmids (GenScript) containing TCR beta and alpha chains under the control of a SFFV promoter and separated by a furin cleavage site and a P2A ribosomal skip sequence. Human TCR variable regions were fused to cysteine-modified mouse constant regions for both chains resulting in recombinant mTCR (Cohen et al., 2006; Kuball et al., 2007). The mTCR-transduced, puromycin-selected (Thermo Fisher) Jurkat cells were expanded in RPMI media supplemented with 10% FBS (Thermo Fisher). Neoantigen reactivity of the mTCR Jurkat cells was determined by IL-2 secretion measured by electrochemiluminescence (MSD) in 24-hour in vitro assays where mTCR transduced Jurkat cells were co-cultured with neoantigen peptide and CD3⁺ T cell-depleted autologous patient PBMCs.

### MultiOmyx Staining

For multiplexed T cell analysis, multiplexing immunofluorescence (IF) analysis on FFPE tumor blocks was performed at NeoGeonomics Laboratories (Lake Forest, CA). Each FFPE slide was presented to a NeoGenomics pathologist for tissue annotation and selection. The areas selected to be tumor-relevant by the pathologist were used for staining and analysis. The staining was performed using a single 4 uM FFPE slide. Slides were prepared and stained using MultiOmyx optimized protocols by Neogeneomics. For each round of staining, conjugated fluorescent antibodies are applied to each slide, followed by image acquisition of stained slides. The dye is then quenched allowing for subsequent rounds of staining with other antibodies. Proprietary deep learning-based workflows were applied to identify individual cells and perform cell classification for all individual markers, as well as to identify tissue and tumor regions for analysis. Individual cell and region classification results were combined to generate co-expression summaries for phenotypes of interest. Antibodies used for staining include CD3, CD4, CD8, Pan CK, DAPI. Analysis was performed according to NeoGeonomics Laboratories' optimized protocols (Gerdes et al., 2013; Xu-Monette et al., 2019).

### Multiplex IHC Staining Protocol

Multiplex immunofluorescence (mIF) staining was performed on 4 μm FFPE tumor sections using the OPAL mIHC kit (Akoya Biosciences). Briefly, sections were deparaffinized and rehydrated. Antigen retrieval was performed in a using an antigen unmasking solution (citrate-based and tris-based). Protein blocking was performed for 10 minutes using Antibody Diluent/Block solution (Akoya Biosciences). Primary antibodies were incubated for 30 minutes at RT. Slides were then incubated with Polymer-HRP(Vector Labs) diluted 1:1 with TBS for 30 minutes. For visualization with OPAL fluors, slides were incubated for 10 minutes with OPAL colors. Another heat step consisting of boiling the slides in antigen unmasking solution was performed to remove the antibody complexes. The same sequence of blocking, primary antibody incubation, Polymer-HRP application and heating was repeated for each antibody in the multiplex sequence. Finally, the slides were counterstained with DAPI, mounted with prolong gold and coverslipped. Some antibodies used in staining panels include CD3, CD4, HLA DP/DQ/DR, and Pan Ck.

### Quantification and Statistical Analysis

### Multispectral Analysis

The multiplex slides were scanned with a Vectra 3.0 microscope system (Perkin Elmer). Whole slide scans were performed using the 10× objective lens. Up to ten individual fields were selected in the Phenochart program (Phenochart 1.0.9, Perkin Elmer) for higher resolution scanning at 20× (669×500 μm). Multispectral images were unmixed and analyzed using inForm software (inForm 2.4.2, Perkin Elmer) and analyzed with tissue component segmentation based on tumor cell staining: tumor=Pan Ck+ regions and stroma=Pan Ck− regions. Individual cells (defined by DAPI positive nuclei) were then phenotyped to characterize various cell populations. Density of cells in each ROI was calculated by combining the cell counts from all images and normalizing by the total area (cells/mm²).

### Single-Cell RNA Sequencing Data Analysis

Cell Ranger version 6.0.1 (10× Genomics) was used to align raw sequencing data with “count” option. Library with RNA was aligned on GRCh38-2020-A genome. TCR library was aligned on vdj_GRCh38_alts_ensembl-5.0.0 assembly. CITE library was aligned on a custom panel of antibodies. R package Seurat 4.0.4 (Stuart et al., 2019) was used for downstream analysis of data acquired from cellranger count. We filtered cells that (1) had more than 25% of mitochondrial gene content, (2) had less than 1024 UMI from RNA or 3) had less than 90 UMI for CITE library. RNA library was log-normalized with a scale factor of 10⁴. The 2000 most variable genes were detected by the FindVariableFeatures function. These 2000 genes were overlaid with 10× Genomics Immunological panel (1056 genes) to de-noise the list, resulting in 344 genes used in downstream analysis. Latent variables—number of UMI's and mitochondrial content—were regressed out using a negative binomial model with function ScaleData. Principle component analysis (PCA) was performed with the RunPCA function. For RNA library harmony function was called (R package Harmony, (Korsunsky et al., 2019)) to remove donor effect. CITE library was normalized using CLR method, scaled (by ScaleData function) using all available antibodies (except for LAG3, TIM3, 41BB, CTLA4 and KLRG1 that had low expression and off-target binding) and PCA was run on it. RNA- and CITE-processed data were used to run WNN method by FindMultiModalNeighbors function to create new modality to run dimensionality reduction and clustering that takes into account RNA and protein levels. A UMAP dimensionality reduction was performed on the WNN using the first 20 PCA components for harmonized RNA and 13 for CITE to obtain a two-dimensional representation of the cell states. For clustering, FindClusters function was used with SLM algorithm and resolution 0.25. To identify marker genes, FindAllMarkers function was used to compare cluster against all other clusters, and FindMarkers to compare selected clusters. For each cluster, only genes that were expressed in more than 15% of cells with at least 0.15-fold differences were considered. For heatmap representation we used mean expression of protein markers inside each cluster. Heatmaps were built with ComplexHeatmap R package (Gu, Eils and Schlesner, 2016)_To analyze TCR all TCR that did not corresponding cell barcode in RNA+CITE library post quality control were filtered out. Gini coefficient was computed with DescTools R package.

### Statistical Analysis

Anti-tumor activity was assessed for objective response rate (ORR), duration of response (DOR), progression free survival (PFS) and overall survival (OS). ORR is defined as the proportion of patients who achieve CR or PR based on RECIST v1.1. DOR is defined as the date of the first documented confirmed response to the date of the first documented PD or death. PFS is defined as the time from the date of first dosing to the date of first documented PD or death. OS is defined from the date of first dosing to death. Statistical efficacy analyses were performed for both the ITT set defined as receiving at least one dose of any study drug, and the safety set defined as receiving at least one dose of vaccine. The Kaplan-Meier method was used to estimate PFS and OS. For PFS, patients who did not have a PFS event on study or who missed 2 consecutive tumor assessments were censored at the time of next scheduled assessment from the last tumor assessment. For OS, patients who were alive or who were lost to follow-up were censored at the last record on database. Statistical analyses of clinical data were performed using SAS (version 9.2). All other analyses were performed in either the R language and environment for statistical computing (version 4.0.0) or GraphPad Prism (version 7.01). Likewise, all FIGs. were created using an R script on the “ggplot2” package (Wickham, 2016) or GraphPad Prism. Unless otherwise specified, p values were derived from a two-tailed Student's t test.

1. A method of treating or preventing a cancer in a human subject in need thereof comprising administering to the human subject in need thereof:
(a) a first component comprising:
(i) a polypeptide comprising a cancer-specific neoepitope of a protein expressed by cancer cells of the cancer,
(ii) a polynucleotide encoding the polypeptide of (i),
(iii) one or more APCs comprising the polypeptide of (i) or the polynucleotide of (ii),
(iv) a T cell receptor (TCR) specific for a complex comprising an HLA protein expressed by the human subject and a cancer-specific neoepitope of a protein expressed by cancer cells of the cancer neoepitope, or
(v) T cells comprising the TCR of (iv); and
(b) a second component comprising an anti-cancer agent which is an antibody or an antigen-binding portion thereof that binds specifically to a Programmed Death-1 (PD-1) receptor and inhibits PD-1 activity; and
(c) a third component comprising a platinum-based chemotherapy;
wherein the human subject:
(i) has not previously received a systemic treatment for metastatic disease,
(ii) has not previously received an immunotherapy with an anti-PD-1 antibody, and
(iii) has not previously received an immunotherapy with an anti-PD-L1 antibody.

2. The method of claim 1, wherein the method comprises administering to the human subject a combination of the second component and the third component prior to administering the first component.

3. The method of claim 2, wherein the method comprises administering to the human subject a combination of the second component and the third component for a period of 12 weeks prior to administering the first component.

4. The method of claim 3, wherein manufacturing of the first component takes place during the period of 12 weeks in which the combination of the second component and the third component are administered.

5. The method of claim 3 or 4, wherein the method comprises administering the first component for a period of 12 weeks after administering the combination of the second component and the third component for a period of 12 weeks.

6. The method of claim 5, wherein the administering the first component for a period of 12 weeks after administering the combination of the second component and the third component for a period of 12 weeks comprises administering the first component at four separate anatomical locations of the human subject.

7. The method of claim 5 or 6, wherein the administering the first component for a period of 12 weeks after administering the combination of the second component and the third component for a period of 12 weeks comprises administering five priming does of the first component and two booster doses of the first component.

8. The method of claim 7, wherein the administering the first component for a period of 12 weeks after administering the combination of the second component and the third component for a period of 12 weeks comprises administering a priming dose of the first component on days 1 and 4 and then weekly in weeks 13, 14, and 15; and administering a boosting dose in weeks 19 and 23.

9. The method of any one of claims 5-8, wherein the second component is administered to the human subject during the period of 12 weeks in which the first component is administered.

10. The method of claim 9, wherein the second component is administered to the human subject after the period of 12 weeks in which the first component and second component are administered.

11. The method of claim 10, wherein the second component is administered to the human subject for a period of at least 28 weeks after the period of 12 weeks in which the first component and second component are administered.

12. The method of claim 11, wherein the second component is administered to the human subject for a period of 80 weeks after the period of 12 weeks in which the first component and second component are administered.

13. The method of any one of claims 2-12, wherein the second component is administered to the human subject for a total period of at least 52 weeks or about 103 or about 104 weeks.

14. The method of any one of claims 2-13, wherein the third component is not administered to the human subject during or after administration of the first component.

15. The method of any one of claims 3-14, wherein the third component is not administered to the human subject following administration of the combination of the second component and the third component for the period of 12 weeks prior to administering the first component.

16. The method of any one of claims 1-15, wherein the human subject has a KRAS mutation, a TP53 mutation, and/or a KEAP1 mutation.

17. The method of claim 16, wherein the cancer-specific neoepitope of the first component does not comprise a KRAS neoepitope, a TP53 neoepitope, and/or a KEAP1 neoepitope.

18. The method of claim 1, wherein the cancer is a lung cancer.

19. The method of claim 18, wherein the lung cancer is non-small cell lung cancer (NSCLC).

20. The method of claim 19, wherein the NSCLC has a squamous histology.

21. The method of claim 19, wherein the NSCLC has a non-squamous histology.

22. The method of claim 19, wherein the NSCLC is metastatic NSCLC.

23. The method of any one of claims 1-22, wherein the first component comprises the polypeptide comprising a cancer-specific neoepitope of a protein expressed by cancer cells of the cancer.

24. The method of claim 23, wherein the first component comprises an adjuvant.

25. The method of claim 24, wherein the adjuvant comprises poly I:poly C.

26. The method of claim 1-25, wherein the cancer-specific neoepitope comprises at least two different cancer-specific neoepitopes of a protein expressed by cancer cells of the cancer.

27. The method of claim 1-25, wherein the cancer-specific neoepitope comprises at most twenty different cancer-specific neoepitopes of a protein expressed by cancer cells of the cancer.

28. The method of any one of claims 1-27, wherein the method comprises comparing: (i) nucleic acid sequences obtained by whole genome sequencing or whole exome sequencing of cancer cells from the single subject to (ii) nucleic acid sequences obtained by whole genome sequencing or whole exome sequencing of non-cancer cells from the single subject.

29. The method of claim 28, wherein the method comprises identifying a plurality of cancer specific nucleic acid sequences that are unique to cancer cells of the human subject based on the comparing.

30. The method of claim 29, wherein the method comprises predicting or calculating binding affinities of cancer-specific neoepitope sequences encoded by the identified plurality of cancer specific nucleic acid sequences to a protein encoded by an HLA allele of the human subject by an HLA peptide binding analysis using a program implemented on a computer system.

31. The method of claim 30, wherein the method comprises selecting at least two cancer-specific neoepitope sequences predicted or calculated to have an IC₅₀ to a protein encoded by an HLA allele of the human subject with of less than 500 nM or 150 nM or less.

32. The method of any one of claims 1-31, wherein the anti-PD-1 antibody or antigen-binding portion thereof cross-competes with nivolumab for binding to human PD-1.

33. The method of any one of claims 1-31, wherein the anti-PD-1 antibody or antigen-binding portion thereof comprises a heavy chain constant region which is of a human IgG1 or IgG4 isotype.

34. The method of any one of claims 1-31, wherein the anti-PD-1 antibody or antigen-binding portion thereof is a chimeric, humanized or human monoclonal antibody or a portion thereof.

35. The method of any one of claims 1-31, wherein the anti-PD-1 antibody is pembrolizumab.

36. The method of claim 35, wherein the anti-PD-1 antibody or antigen-binding portion thereof is administered at a dose ranging from 0.1 to 10.0 mg/kg body weight once every 2, 3 or 4 weeks.

37. The method of claim 36, wherein the anti-PD-1 antibody or antigen-binding portion thereof is administered at a dose of 5 or 10 mg/kg body weight once every 3 weeks.

38. The method of claim 36, wherein the anti-PD-1 antibody or antigen-binding portion thereof is administered at a dose of 3 mg/kg body weight once every 2 weeks.

39. method of claim 35, wherein the anti-PD-1 antibody or antigen-binding portion thereof is administered via intravenous infusion at a dose of 200 mg on cycle day 1 every 3 weeks.

40. The method of any one of claims 1-39, wherein 1, wherein the platinum-based chemotherapy is a platinum-based doublet chemotherapy (PT-DC).

41. The method of claim 40, wherein the PT-DC is a combination of pemetrexed and carboplatin.

42. The method of claim 41, wherein the carboplatin is administered at a dose to achieve an area under the free carboplatin plasma concentration versus time curve (AUC) of 5.

43. The method of claim 41 or 42, wherein the pemetrexed is administered at a dose of 500 mg/m\hat{ }2.

44. The method of any one of claims 40-43, wherein the PT-DC was administered concurrently with the anti-PD-1 antibody or antigen-binding portion thereof for 4 doses of the anti-PD-1 antibody or antigen-binding portion thereof, followed by repeated administration of the anti-PD-1 antibody or antigen-binding portion thereof alone.

45. The method of any one of claims 1-44, wherein the method promotes epitope spread.

46. The method of any one of claims 1-44, wherein the method promotes epitope spread of an epitope that is different than any of the cancer-specific neoepitopes.

47. The method of claim 46, wherein the epitope that is different than any of the cancer-specific neoepitopes comprises a KRAS neoepitope, a TP53 neoepitope, and/or a KEAP1 neoepitope.

48. The method of claim 47, wherein the KRAS neoepitope comprises a G12C or G12V mutation.

49. The method of any one of claims 1-44, wherein a median progression-free survival (PFS) of a first population of human subjects with the cancer treated with the first, second and third components is longer than a median PFS of a second population of subjects with the cancer treated with the second and/or third components but not the first component.

50. The method of any one of claims 1-44, wherein an overall response rate (ORR) of a first population of human subjects with the cancer treated with the first, second and third components is higher than an ORR of a second population of subjects with the cancer treated with the second and/or third components but not the first component.

51. The method of any one of claims 1-44, wherein a percentage of subjects with at least a 9 or 12-month progression-free survival (PFS) of a first population of human subjects with the cancer treated with the first, second and third components is higher than a percentage of subjects with at least a 9 or 12-month PFS of a second population of subjects with the cancer treated with the second and/or third components but not the first component.

52. The method of any one of claims 1-44, wherein a median overall survival (OS) of a first population of human subjects with the cancer treated with the first, second and third components is longer than a median OS of a second population of subjects with the cancer treated with the second and/or third components but not the first component.

53. The method of any one of claims 1-44, wherein a percentage of subjects who achieve complete response, partial response, prolonged stable disease or stable disease for 6 months or more (CBR) of a first population of human subjects with the cancer treated with the first, second and third components is higher than an CBR of a second population of subjects with the cancer treated with the second and/or third components but not the first component.

54. The method of any one of claims 1-44, wherein reduction of tumor size in a first population of human subjects with the cancer treated with the first, second and third components is greater than reduction of tumor size in a second population of subjects with the cancer treated with the second and/or third components but not the first component.

55. The method of any one of claims 1-44, wherein a level of CD4+ T cells that infiltrate a tumor in a first population of human subjects treated with the first, second and third components is higher than a level of CD4+ T cells that infiltrate a tumor in a second population of subjects treated with the second and/or third components but not the first component.

56. The method of any one of claims 1-44, wherein a level of effector and cytotoxic CD4+ T cells generated in a first population of human subjects treated with the first, second and third components is higher than a level of effector and cytotoxic CD4+ T cells generated in a second population of subjects treated with the second and/or third components but not the first component.

57. The method of any one of claims 1-44, wherein the method increases a level of CD4+ T cells specific to a cancer-specific neoepitope that upregulate expression of ZEB2, PDCD1, TOX, TIGT, CXCR3, ITGB1, GZMA and/or ICOS.

58. The method of any one of claims 1-44, wherein the method increases a level of CD4+/CD62L^{hi}/CD69+/CD27+/CCR7+ T cells specific to a cancer-specific neoepitope.

59. The method of any one of claims 1-44, wherein the method increases a level of CD4+/NKG7+/CCL4+/CCL5+/GNLY+/LAG3+ T cells specific to a cancer-specific neoepitope.

60. The method of any one of claims 1-59, wherein the human subject is identified as having a PD-L1-positive cancer prior to administration of the first, second and/or third components.

61. A method of treating or preventing a cancer in a human subject in need thereof comprising administering to the human subject in need thereof:
(a) a first component comprising:
(i) a polypeptide comprising a cancer-specific neoepitope of a protein expressed by cancer cells of the cancer,
(ii) a polynucleotide encoding the polypeptide of (i),
(iii) one or more APCs comprising the polypeptide of (i) or the polynucleotide of (ii),
(iv) a T cell receptor (TCR) specific for a complex comprising an HLA protein expressed by the human subject and a cancer-specific neoepitope of a protein expressed by cancer cells of the cancer neoepitope, or
(v) T cells comprising the TCR of (iv); and
(b) a second component comprising an anti-cancer agent which is an antibody or an antigen-binding portion thereof that binds specifically to a Programmed Death-1 (PD-1) receptor and inhibits PD-1 activity; and
(c) a third component comprising a platinum-based chemotherapy; wherein:
(i) the method promotes epitope spread of an epitope that is different than any of the cancer-specific neoepitopes;
(ii) a median progression-free survival (PFS) of a first population of human subjects with the cancer treated with the first, second and third components is longer than a median PFS of a second population of subjects with the cancer treated with the second and/or third components but not the first component;
(iii) an overall response rate (ORR) of a first population of human subjects with the cancer treated with the first, second and third components is higher than an ORR of a second population of subjects with the cancer treated with the second and/or third components but not the first component;
(iv) a percentage of subjects with at least a 9 or 12-month progression-free survival (PFS) of a first population of human subjects with the cancer treated with the first, second and third components is higher than a percentage of subjects with at least a 9 or 12-month PFS of a second population of subjects with the cancer treated with the second and/or third components but not the first component;
(v) a median overall survival (OS) of a first population of human subjects with the cancer treated with the first, second and third components is longer than a median OS of a second population of subjects with the cancer treated with the second and/or third components but not the first component;
(vi) a percentage of subjects who achieve complete response, partial response, prolonged stable disease or stable disease for 6 months or more (CBR) of a first population of human subjects with the cancer treated with the first, second and third components is higher than an CBR of a second population of subjects with the cancer treated with the second and/or third components but not the first component;
(vii) reduction of tumor size in a first population of human subjects with the cancer treated with the first, second and third components is greater than reduction of tumor size in a second population of subjects with the cancer treated with the second and/or third components but not the first component;
(viii) a level of CD4+ T cells that infiltrate a tumor in a first population of human subjects treated with the first, second and third components is higher than a level of CD4+ T cells that infiltrate a tumor in a second population of subjects treated with the second and/or third components but not the first component;
(ix) a level of effector and cytotoxic CD4+ T cells generated in a first population of human subjects treated with the first, second and third components is higher than a level of effector and cytotoxic CD4+ T cells generated in a second population of subjects treated with the second and/or third components but not the first component;
(x) the method increases a level of CD4+ T cells specific to a cancer-specific neoepitope that upregulate expression of ZEB2, PDCD1, TOX, TIGT, CXCR3, ITGB1, GZMA and/or ICOS;
(xi) the method increases a level of CD4+/CD62L^{hi}/CD69+/CD27+/CCR7+ T cells specific to a cancer-specific neoepitope; and/or
(xii) the method increases a level of CD4+/NKG7+/CCL4+/CCL5+/GNLY+/LAG3+ T cells specific to a cancer-specific neoepitope.

62. The method of claim 61, wherein the human subject is identified as having a PD-L1-positive cancer prior to administration of the first, second and/or third components.

63. The method of claim 61, wherein the human subject has not previously received a systemic treatment for metastatic disease, has not previously received an immunotherapy with an anti-PD-1 antibody, and/or has not previously received an immunotherapy with an anti-PD-L1 antibody.
